(19) United States
(12) Patent Application Publication
(10) Pub. No.: US 2021/0070869 A1
(43) Pub. Date: Mar. 11, 2021

(54) AXL-SPECIFIC ANTIBODIES FOR CANCER TREATMENT

(71) Applicant: GENMAB A/S, Copenhagen V (DK)

(72) Inventors: Maarten JANMAAT, Utrecht (NL); Esther BREIJ, Utrecht (NL); Ulf FORSSMANN, Hannover (DE); Tahamtan AHMADI, Rydal, PA (US); Julia BOSHUIZEN, Amsterdam (NL); Daniel PEEPER, Amstelveen (NL); Nora PENCHEVA, Utrecht (NL)

(21) Appl. No.: 17/046,199

(22) PCT Filed: Apr. 10, 2019
(86) PCT No.: PCT/EP2019/059171

§ 371 Date: Oct. 8, 2020

Related U.S. Application Data
(60) Provisional application No. 62/655,417, filed on Apr. 10, 2018.

(51) Int. Cl. C07K 16/28; A61K 47/68; A61P 35/00

(52) U.S. Cl. CPC C07K 16/2863; A61K 47/6849; A61P 35/00; C07K 16/2827; A61K 47/6803; C07K 16/2818

(57) ABSTRACT

The disclosure relates to anti-AXL antibodies, immunoconjugates, and compositions for treatment of cancer, which is resistant to or is predicted to be or become resistant to treatment with a programmed cell death-1/programmed cell death-1 ligand (PD-1/PD-L1) inhibitor.

### FIELD OF INVENTION

The present invention relates to the use of antibodies binding AXL, immunoconjugates, and compositions comprising such antibodies or immunoconjugates; in particular the use of said antibodies and immunoconjugates for treatment of patients, who have failed to respond to anti-PD-1/PD-L1 treatment or have not responded satisfactorily to such treatment.

### BACKGROUND

AXL is a 104-140 kDa transmembrane protein which belongs to the TAM subfamily of mammalian Receptor Tyrosine Kinases (RTKs) and which has transforming abilities (Paccez et al., 2014). The AXL extracellular domain is composed of a combination of two membrane-distal N-terminal immunoglobulin (Ig)-like domains (Ig1 and Ig2 domains) and two membrane-proximal fibronectin type III (FNIII) repeats (the FN1- and FN2-domains) (Paccez et al., 2014). Enhanced or de novo expression of AXL has been reported in a variety of cancers, including gastric, prostate, ovarian, and lung cancer (Paccez et al., 2014).

AXL can be activated upon binding of its ligand, the vitamin K-dependent growth arrest-specific factor 6 (Gas6). Gas6-binding to AXL leads to AXL dimerization, autophosphorylation and subsequent activation of intracellular signaling pathways, such as the PI3K/AKT, mitogen-activated protein kinase (MAPK), STAT and NE-KB cascades (Leconet et al., 2013). In cancer cells, AXL expression has been associated with tumor cell motility, invasion, migration, and is involved in epithelial-to-mesenchymal transition (EMT) (Linger et al., 2010).

Targeted inhibition of AXL and/or its ligand Gas6 may be effective as anti-tumor therapy using, e.g., small molecules or anti-AXL antibodies (Linger et al., 2010). Anti-AXL antibodies have been described that attenuate NSCLC and breast cancer xenograft growth in vivo by downregulation of receptor expression, reducing tumor cell proliferation and inducing apoptosis (Li et al., 2009; Ye et al., 2010 (a);

WO 2011/159980, Genentech). Various other anti-AXL antibodies have also been reported (Leconet et al., 2013; Iida et al., 2014; WO 2012/175691, INSERM; WO 2012/175692, INSERM; WO 2013/064685, Pierre Fabre Medicaments; WO 2013/090776, INSERM; WO 2009/063965, Chugai Pharmaceuticals and WO 2010/131733), including an ADC based on an anti-AXL antibody and a pyrrolobenzo-diazepine (PBD) dimer (WO 2014/174111, Pierre Fabre Medicament and Spirogen Sarl).

Programmed death 1 (PD-1) is a type I membrane protein of 268 amino acids. PD-1 is a member of the extended CD28/CTLA-4 family of T cell regulators and it is suggested that PD-1 and its ligands negatively regulate immune responses. PD-L1 is the ligand for PD1; it is highly expressed in several cancers and the role of PD1 in cancer immune evasion is well established. Recently, a number of cancer immunotherapy agents which target the PD-1 and/or PDL-1 have been developed (Sunshine & Taube, 2015). While inti-PD1/PD-L1 therapy has been claimed to be among the most effective anti-cancer immunotherapies available, it has been shown that as many as 60% of patients receiving such therapy display primary resistance. Furthermore, the development of acquired resistance in melanoma patients with an objective response to anti-PD1 therapy has also been reported (O'Donnell et al., 2016). Since little is known regarding the mechanisms responsible for resistance in patients receiving anti-PD1 therapy, few effective therapeutic options are available for such patients.

Hence, there is a need for improved methods of treating cancers which are, or which are predicted to be or become, resistant to treatment with PD-1/PD-L1 inhibitors.

### SUMMARY OF THE INVENTION

It is an object of the present invention to provide cancer therapy for subjects with resistance to or subjects that are predicted to be or become resistant to treatment with of the interaction between a programmed cell death-1 (PD-1) receptor and a PD-1 receptor ligand.

In a first aspect, the invention provides an antibody binding to human AXL or an antibody-drug conjugate (ADC) comprising said antibody, for use in treating cancer in a subject, wherein
said cancer is resistant to or is predicted to be or become resistant to;
said cancer has failed to respond to, or is predicted to fail to respond to; and/or
said subject has relapsed after or is predicted to relapse after treatment with an inhibitor of the interaction between a programmed cell death-1 (PD-1) receptor and its ligand.

In a second aspect, the invention provides an antibody binding to human AXL or an antibody-drug conjugate (ADC) comprising an antibody binding to human AXL, for use in the manufacture of a medicament for treating cancer in a subject, wherein
said cancer is resistant to or is predicted to be or become resistant to;
said cancer has failed to respond to, or is predicted to fail to respond to; and/or
said subject has relapsed after or is predicted to relapse after
   treatment with an inhibitor of the interaction between a programmed cell death-1 (PD-1) receptor and its ligand.

A third aspect of the invention provides a method of treating cancer in a subject, wherein said cancer
is resistant to or is predicted to be or become resistant to;
has failed to respond to, or is predicted to fail to respond to; and/or
has relapsed after or is predicted to relapse after
   treatment with an inhibitor of the interaction between a programmed cell death-1 (PD-1) receptor and its ligand. The method comprises administering to said subject a therapeutically effective amount of an antibody binding to human AXL or an antibody-drug conjugate (ADC) comprising an antibody binding to human AXL.

### LEGENDS TO THE FIGURES

FIG. 1. Anti-tumor efficacy of IgG1-AXL-107-vcMMAE in the melanoma xenograft model SkMel147 in the presence of tumor-specific, human T-cells, as described in Example 5. Average tumor size after injection of mice with control T cells or MART-1 T cells, in combination with IgG1-b12-vcMMAE (Ctrl ADC), IgG1-AXL-107-vcMMAE, or IgG1-b12-vcMMAE and anti-PD-1 (pembrolizumab). Error bars show the standard error of the mean (SEM).

FIG. 2. Kaplan-Meyer graph showing the survival (tumor size cutoff >500 mm3) of the mice in the different groups in the SkMel147 model, as described in Example 5.

FIG. 3. Tumor size in selected mice from the melanoma xenograft model SkMel147 that were sequentially treated with IgG1-AXL-107-vcMMAE, as described in Example 5. Tumor size in mice initially injected with (A) control T cells and control ADC (n=5), (B) MART-1 T cells and control ADC (n=2), and (C) MART-1 T cells, control ADC and anti-PD-1 (n=2) were treated with 4 mg/kg IgG1-AXL-107-vcMMAE on the day indicated with the arrow. Tumor size per mouse is plotted.

FIG. 4. Anti-tumor efficacy of IgG1-AXL-107-vcMMAE in the melanoma xenograft model BLM in the presence of tumor-specific, human T-cells, as described in Example 6. Average tumor size after injection of mice with control T cells or MART-1 T cells, in combination with IgG1-b12-vcMMAE (Ctrl ADC), IgG1-AXL-107-vcMMAE, or IgG1-b12-vcMMAE and anti-PD-1 (pembrolizumab). Error bars show the standard error of the mean (SEM).

FIG. 5. Kaplan-Meyer graph showing the survival (tumor size cutoff >500 mm3) of the mice in the different groups in the BLM model, as described in Example 6.

FIG. 6: Design of phase 2 study including dose excalation and expansion.

FIG. 7: Design of 1Q3W dosage regimen: Dosing once every 3 weeks.

FIG. 8: Design of 3Q4W dosage regimen: Weekly dosing for 3 weeks followed by one treatment-free week.

FIG. 9: Subject 403 lesion snapshots.

### DETAILED DESCRIPTION

### Definitions

In a first aspect, the present invention provides an antibody binding to human AXL or an antibody-drug conjugate (ADC) comprising an antibody binding to human AXL as defined in any aspect or embodiment herein, for use in treating cancer in a subject. In particular the antibody or ADC is for use in treating cancer in which prior treatment has not been effective

The term “AXL” or “Axl” as used herein, refers to the protein entitled AXL, which is also referred to as UFO or JTK11, a 894 amino acid protein with a molecular weight of 104-140 kDa that is part of the subfamily of mammalian TAM Receptor Tyrosine Kinases (RTKs). The molecular weight is variable due to potential differences in glycosylation of the protein. The AXL protein consists of two extracellular immunoglobulin-like (Ig-like) domains on the N-terminal end of the protein, two membrane-proximal extracellular fibronectin type III (FNIII) domains, a transmembrane domain and an intracellular kinase domain. AXL is activated upon binding of its ligand Gas6, by ligand-independent homophilic interactions between AXL extracellular domains, by autophosphorylation in presence of reactive oxygen species (Korshunov et al., 2012) or by transactivation through EGFR (Meyer et al., 2013), and is aberrantly expressed in several tumor types. In humans, the AXL protein is encoded by a nucleic acid sequence encoding the amino acid sequence shown in SEQ ID NO:130 (human AXL protein: Swissprot P30530). For cynomolgus AXL protein, see Genbank accession HB387229.1 (SEQ ID NO:147).

The term “antibody” as used herein is intended to refer to an immunoglobulin molecule, a fragment of an immunoglobulin molecule, or a derivative of either thereof, which has the ability to specifically bind to an antigen under typical physiological and/or tumor-specific conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to be internalized). The binding region (or binding domain which may be used herein, both having the same meaning) which interacts with an antigen, comprises variable regions of both the heavy and light chains of the immunoglobulin molecule. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody as used herein, unless otherwise stated or clearly contradicted by context, includes fragments of an antibody that retain the ability to specifically interact, such as bind, to the antigen. It has been shown that the antigen-binding function of an antibody may be performed by fragments of a full-length antibody. Examples of binding fragments encompassed within the term “antibody” include (i) a Fab′ or Fab fragment, a monovalent fragment consisting of the VL, VH, CL and CH1 domains, or a monovalent antibody as described in WO 2007/059782; (ii) F(ab′)2 fragments, bivalent fragments comprising two Fab fragments linked by a disulfide bridge at the hinge region; (iii) an Fd fragment consisting essentially of the VH and CH1 domains; (iv) an Fv fragment consisting essentially of the VL and VH domains of a single arm of an antibody, (v) a dAb fragment (Ward et al., 1989), which consists essentially of a VH domain and is also called domain antibody (Holt et al., 2003); (vi) camelid or nanobodies (Revets et al., 2005) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al. (1988) and Huston et al. (1988). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention are discussed further herein. It also should be understood that the term antibody, unless specified otherwise, also includes polyclonal antibodies, monoclonal antibodies (mAbs), antibody-like polypeptides, such as chimeric antibodies and humanized antibodies, as well as ‘antibody fragments’ or ‘fragments thereof’ retaining the ability to specifically bind to the antigen (antigen-binding fragments) provided by any known technique, such as enzymatic cleavage, peptide synthesis, and recombinant techniques, and retaining the ability to be conjugated to a toxin. An antibody as generated can possess any isotype.

The term “an inhibitor of the interaction between a programmed cell death-1 (PD-1) receptor and its ligand” refers broadly to any agent which is agent which is capable of inhibiting (e.g. reducing or abolishing) the interaction between the programmed cell death-1 (PD-1) receptor, such as the human programmed cell death-1 (PD-1) receptor and at least one of its ligands. In particular, the term includes such an agent, which is capable of reducing or abolishing any of the responses to activation of the PD-1 receptor, including the inhibition of T lymphocyte proliferation, the survival and effector functions (cytotoxicity, cytokine release), the induction of apoptosis of tumor-specific T cells, the promotion of differentiation of CD4+ T cells into Foxp3+ regulatory T cells, and/or the resistance of tumor cells to cytotoxic T-lymphocyte (CTL) attack.

The term “an inhibitor of the interaction between a programmed cell death-1 (PD-1) receptor and its ligand” also includes the commonly used term “PD-1/PD-L1 inhibitor”.

The term “Growth Arrest-Specific 6” or “Gas6” as used herein, refers to a 721 amino acid protein, with a molecular weight of 75-80 kDa, that functions as a ligand for the TAM family of receptors, including AXL. Gas6 is composed of an N-terminal region containing multiple gamma-carboxyglutamic acid residues (Gla), which are responsible for the specific interaction with the negatively charged phospholipid membrane. Although the Gla domain is not necessary for binding of Gas6 to AXL, it is required for activation of AXL. Gas6 may also be termed as the “ligand to AXL”.

When used herein in the context of an antibody and a Gas6 ligand or in the context of two or more antibodies, the term “competes with” or “cross-competes with” indicates that the antibody competes with the ligand or another antibody, e.g., a “reference” antibody in binding to an antigen, respectively. Example 2 of WO 2016/005593 A1 (Genmab) describes an example of how to test competition of an anti-AXL antibody with the AXL-ligand Gas6. Preferred reference antibodies for cross-competition between two antibodies are those comprising a binding region comprising the VH region and VL region of an antibody herein designated 107, 148, 733, 154, 171, 183, 613, 726, 140, 154-M103L, 172, 181, 183-N52Q, 187, 608-01, 610-01, 613-08, 620-06 or 726-M101L, as set forth in Table 2. A particularly preferred reference antibody is the antibody designated 107.

The term “immunoglobulin” as used herein is intended to refer to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized (see for instance Fundamental Immunology Ch. 7 (Paul, W., ed., 2nd ed. Raven Press, N.Y. (1989). Within the structure of the immunoglobulin, the two heavy chains are inter-connected via disulfide bonds in the so-called “hinge region”. Equally to the heavy chains each light chain is typically comprised of several regions; a light chain variable region (abbreviated herein as VL region) and a light chain constant region. Furthermore, the VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4. CDR sequences are defined according to IMGT (see Lefranc et al. (1999) and Brochet et al. (2008)).

The term “immunoglobulin heavy chain” or “heavy chain of an immunoglobulin” as used herein is intended to refer to one of the heavy chains of an immunoglobulin. A heavy chain is typically comprised of a heavy chain variable (abbreviated herein as VH) region and a heavy chain constant region (abbreviated herein as CH) which defines the isotype of the immunoglobulin. The heavy chain constant region typically is comprised of three domains, CH1, CH2, and CH3.

The term “immunoglobulin light chain” or “light chain of an immunoglobulin” as used herein is intended to refer to one of the light chains of an immunoglobulin. A light chain is typically comprised of a light chain variable (abbreviated herein as VL) region and a light chain constant region (abbreviated herein as CL). The light chain constant region typically is comprised of one domain, CL.

The terms “monoclonal antibody”, “monoclonal Ab”, “monoclonal antibody composition”, “mAb”, or the like, as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be produced by a hybridoma which includes a B cell obtained from a transgenic or transchromosomal non-human animal, such as a transgenic mouse, having a genome comprising a human heavy chain transgene and a light chain transgene, fused to an immortalized cell.

The term “full-length antibody” when used herein, refers to an antibody (e.g., a parent or variant antibody) which contains all heavy and light chain constant and variable domains corresponding to those that are normally found in a wild-type antibody of that isotype.

As used herein, “isotype” refers to the immunoglobulin class (for instance IgG1, IgG2, IgG3, IgG4, IgD, IgA, IgE, or IgM) that is encoded by heavy chain constant region genes.

The term “antigen-binding region” or “binding region” as used herein, refers to a region of an antibody which is capable of binding to the antigen. The antigen can be in solution, adhered to or bound to a surface or, e.g., present on a cell, bacterium, or virion. The terms “antigen” and “target” may, unless contradicted by the context, be used interchangeably in the context of the present invention.

The term “epitope” means a protein determinant capable of specific binding to an antibody. Epitopes usually consist of surface groupings of molecules such as amino acids, sugar side chains or a combination thereof and usually have specific three dimensional structural characteristics, as well as specific charge characteristics. Conformational and non conformational epitopes are distinguished in that the binding to the former but not the latter is lost in the presence of denaturing solvents. The epitope may comprise amino acid residues which are directly involved in the binding, and other amino acid residues, which are not directly involved in the binding, such as amino acid residues which are effectively blocked or covered by the specific antigen binding peptide (in other words, the amino acid residue is within the footprint of the specific antigen binding peptide).

The term “binding” as used herein refers to the binding of an antibody to a predetermined antigen or target, typically with a binding affinity corresponding to a K_{D} of about 10⁻⁶ M or less, e.g. 10⁻⁷ M or less, such as about 10⁻⁸ M or less, such as about 10⁻⁹ M or less, about 10⁻¹° M or less, or about 10⁻¹¹ M or even less when determined by for instance surface plasmon resonance (SPR) technology in a BIAcore 3000 instrument using the antigen as the ligand and the protein as the analyte, and binds to the predetermined antigen with an affinity corresponding to a K_{D} that is at least ten-fold lower, such as at least 100 fold lower, for instance at least 1,000 fold lower, such as at least 10,000 fold lower, for instance at least 100,000 fold lower than its affinity for binding to a non-specific antigen (e.g., BSA, casein) other than the predetermined antigen or a closely-related antigen. The amount with which the affinity is lower is dependent on the K_{D} of the protein, so that when the K_{D} of the protein is very low (that is, the protein is highly specific), then the amount with which the affinity for the antigen is lower than the affinity for a non-specific antigen may be at least 10,000 fold. The term “K_{D}” (M), as used herein, refers to the dissociation equilibrium constant of a particular antibody-antigen interaction, and is obtained by dividing k_{d} by kₐ.

The term “k_{d}” (sec⁻¹), as used herein, refers to the dissociation rate constant of a particular antibody-antigen interaction. Said value is also referred to as the k_{off} value.

The term “kₐ” (M⁻¹×sec⁻¹), as used herein, refers to the association rate constant of a particular antibody-antigen interaction.

The term “K_{D}” (M), as used herein, refers to the dissociation equilibrium constant of a particular antibody-antigen interaction.

The term “K_{A}” (M⁻¹), as used herein, refers to the association equilibrium constant of a particular antibody-antigen interaction and is obtained by dividing the kₐ by the k_{d}.

The term “internalized” or “internalization” as used herein, refers to a biological process in which molecules such as the AXL-ADC are engulfed by the cell membrane and drawn into the interior of the cell. It may also be referred to as “endocytosis”. The internalization of an antibody can, for example, be evaluated according to the assay described in Example 16 of WO 2016/005593 A1.

The terms “antibody binding AXL”, “AXL-antibody” or “anti-AXL antibody” as used herein, refers to any antibody binding an epitope on the extracellular part of AXL.

In the context of the present invention, the term “ADC” refers to an antibody drug conjugate, which in the context of the present invention refers to an anti-AXL antibody which is coupled to a therapeutic moiety, e.g., a cytotoxic moiety as described in the present application. It may e.g. be coupled with a linker to e.g. cysteine or with other conjugation methods to other amino acids. The moiety may e.g. be a drug or a toxin or the like.

As used herein, a “therapeutic moiety” means a compound which exerts a therapeutic or preventive effect when administered to a subject, particularly when delivered as an ADC as described herein. A “cytotoxic” or “cytostatic” moiety is a compound that is detrimental to (e.g., kills) cells. Some cytotoxic or cytostatic moieties for use in ADCs are hydrophobic, meaning that they have no or only a limited solubility in water, e.g., 1 g/L or less (very slightly soluble), such as 0.8 g/L or less, such as 0.6 g/L or less, such as 0.4 g/L or less, such as 0.3 g/L or less, such as 0.2 g/L or less, such as 0.1 g/L or less (practically insoluble). Exemplary hydrophobic cytotoxic or cytostatic moieties include, but are not limited to, certain microtubulin inhibitors such as auristatin and its derivatives, e.g., MMAF and MMAE.

The abbreviation “MMAE” refers to monomethyl auristatin E.

The abbreviation “PAB” refers to the self-immolative spacer:

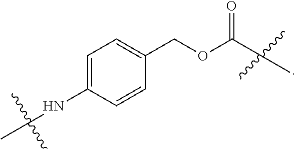 [SMILES: *Nc1ccc(COC(*)=O)cc1]

The abbreviation “MC” refers to the stretcher maleimidocaproyl:

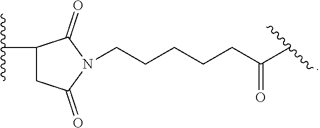 [SMILES: *C(=O)CCCCCN1C(=O)CC(*)C1=O]

“Treatment” refers to the administration of an effective amount of a therapeutically active compound as described herein to a subject with the purpose of easing, ameliorating, arresting or eradicating (curing) symptoms or disease states of the subject.

As used herein, the term “subject” is typically a human to whom an antibody binding to AXL or an ADC comprising such antibody is administered, and who may benefit from the administration of the antibody binding to AXL or the ADC comprising such antibody, including for instance human patients diagnosed as having a cancer that may be treated by killing of AXL-expressing cells, directly or indirectly.

An “effective amount” or “therapeutically effective amount” refers to an amount effective, at dosages and for periods of time necessary, to achieve a desired therapeutic result. A therapeutically effective amount of an AXL-ADC may vary according to factors such as the disease state, age, sex, and weight of the individual, and the ability of the AXL-ADC to elicit a desired response in the individual. A therapeutically effective amount is also one in which any toxic or detrimental effects of the AXL-ADC are outweighed by the therapeutically beneficial effects.

As used herein, a “resistant”, cancer, tumor or the like, means a cancer or tumor in a subject, wherein the cancer or tumor did not respond to treatment with a therapeutic agent from the onset of the treatment (herein referred to as “native resistance”) or initially responded to treatment with the therapeutic agent but became non-responsive or less responsive to the therapeutic agent after a certain period of treatment (herein referred to as “acquired resistance”), resulting in progressive disease. For solid tumors, also an initial stabilization of disease represents an initial response. Other indicators of resistance include recurrence of a cancer, increase of tumor burden, newly identified metastases or the like, despite treatment with the therapeutic agent. Whether a tumor or cancer is, or has a high tendency of becoming, resistant to a therapeutic agent can be determined by a person of skill in the art. For example, the National Comprehensive Cancer Network (NCCN, www.nccn.org) and European Society for Medical Oncology (ESMO, www.esmo.org/Guidelines) provide guidelines for assessing whether a specific cancer responds to treatment.

As used herein, a cancer which is predicted to be or become resistant resistance to a therapeutic agent is a cancer which is known to be associated with a high tendency and/or frequency of being or becoming resistant or refractory to treatment with the therapeutic agent or to the class of drugs to which the therapeutic agent belongs. Likewise, a cancer, which is predicted to fail to respond to treatment with a therapeutic agent is a cancer which is known to be associated with a high tendency and/or frequency of failing to respond to treatment with the therapeutic agent or to the class of drugs to which the therapeutic agent belongs. A subject, which is predicted to relapse after treatment with a therapeutic agent is a patient with a cancer which is known to be associated with a high tendency and/or frequency of relapse after treatment with the therapeutic agent or with an agent of the class of drugs to which the therapeutic agent belongs.

The present invention also provides, in one embodiment, the use of antibodies comprising functional variants of the VL region, VH region, or one or more CDRs of the antibodies described herein. A functional variant of a VL, VH, or CDR used in the context of an anti-AXL antibody still allows the antibody to retain at least a substantial proportion (at least about 50%, 60%, 70%, 80%, 90%, 95% or more) of the affinity/avidity and/or the specificity/selectivity of the parent antibody and in some cases such an anti-AXL antibody may be associated with greater affinity, selectivity and/or specificity than the parent antibody.

Such functional variants typically retain significant sequence identity to the parent antibody. The percent identity between two sequences is a function of the number of identical positions shared by the sequences (i.e., % homology=#of identical positions/total #of positions×100), taking into account the number of gaps, and the length of each gap, which needs to be introduced for optimal alignment of the two sequences. The comparison of sequences and determination of percent identity between two sequences may be accomplished using a mathematical algorithm, as described in the non-limiting examples below.

The term “isotype” as used herein refers to the immunoglobulin class (for instance IgG1, IgG2, IgG3, IgG4, IgD, IgA, IgE, or IgM) or any allotypes thereof, such as IgG1m(za) and IgG1m(f)) that is encoded by heavy chain constant region genes. Further, each heavy chain isotype can be combined with either a kappa (κ) or lambda (λ) light chain.

The term “full-length antibody” when used herein, refers to an antibody (e.g., a parent or variant antibody) which contains all heavy and light chain constant and variable domains corresponding to those that are normally found in a wild-type antibody of that isotype. A full-length antibody according to the present invention may be produced by a method comprising the steps of (i) cloning the CDR sequences into a suitable vector comprising complete heavy chain sequences and complete light chain sequence, and (ii) expressing the complete heavy and light chain sequences in suitable expression systems. It is within the knowledge of the skilled person to produce a full-length antibody when starting out from either CDR sequences or full variable region sequences. Thus, the skilled person would know how to generate a full-length antibody for use according to the present invention.

The percent identity between two nucleotide sequences may be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences may also be determined using the algorithm of E. Meyers and W. Miller, Comput. Appl. Biosci 4, 11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444 453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The term “amino acid substitution” embraces a substitution into any one or the other nineteen natural amino acids, or into other amino acids, such as non-natural amino acids. For example, an amino acid may be substituted for another conservative or non-conservative amino acid. Amino acid residues may also be divided into classes defined by alternative physical and functional properties. Thus, classes of amino acids may be reflected in one or both of the following lists:

Amino acid residue of conservative class:

### Acidic Residues: D and E

### Basic Residues: K, R, and H

### Hydrophilic Uncharged Residues: S, T, N, and Q

### Aliphatic Uncharged Residues: G, A, V, L, and I

### Non-polar Uncharged Residues: C, M, and P

### Aromatic Residues: F, Y, and W

Alternative Physical and Functional Classifications of Amino Acid Residues:

Alcohol group-containing residues: S and T
Aliphatic residues: I, L, V, and M
Cycloalkenyl-associated residues: F, H, W, and Y
Hydrophobic residues: A, C, F, G, H, I, L, M, R, T, V, W, and Y
Negatively charged residues: D and E
Polar residues: C, D, E, H, K, N, Q, R, S, and T
Positively charged residues: H, K, and R
Small residues: A, C, D, G, N, P, S, T, and V
Very small residues: A, G, and S
Residues involved in turn formation: A, C, D, E, G, H, K, N, Q, R, S, P, and T
Flexible residues: Q, T, K, S, G, P, D, E, and R

The terms “lyophilized” and “freeze-dried” are used interchangeably herein and refer to a material that is dehydrated by first freezing and then reducing the surrounding pressure to allow the frozen water in the material to sublimate.

The term “buffer” as used herein denotes a pharmaceutically acceptable buffer. The term “buffer” encompasses those agents which maintain the pH value of a solution, e.g., in an acceptable range and includes, but is not limited to, histidine, citrate, MES, phosphate, TRIS® (tris (hydroxymethyl)aminomethane), carbonic acid, succinate, glycolate and the like, as described herein. Generally, the “buffer” as used herein has a pKa and buffering capacity suitable for the pH range of about 5 to about 7, preferably of about 5.5 to 6.5, preferably about 5.8 to 6.2, such as about pH 6 or about pH 6.0.

The term “bulking agent” includes agents that can provide additional structure to a freeze-dried product (e.g., to provide a pharmaceutically acceptable cake). Commonly used bulking agents include mannitol, glycine, and the like. In addition to providing a pharmaceutically acceptable cake, bulking agents also typically impart useful qualities to the lyophilized composition such as modifying the collapse temperature, providing freeze-thaw protection, further enhancing the protein stability over long-term storage, and the like. These agents can also serve as tonicity modifiers.

The term “stabilizer” as used herein includes agents that provide stability to a protein, e.g., serving as a cryoprotectant during freezing and/or a lyoprotectant during a (freeze-) drying or ‘dehydration’ process. Suitable stabilizers include non-reducing sugars or saccharides and sugar alcohols such as sucrose, trehalose, mannitol, xylitol and the like, as well as amino acids such as glycine, alanine and lysine. Stabilizers can also serve as bulking agents, tonicity-modifying and/or viscosity-increasing agents.

A “surfactant” as used herein is a compound that is typically used in pharmaceutical formulations to prevent drug adsorption to surfaces and or aggregation. Furthermore, surfactants lower the surface tension (or interfacial tension) between two liquids or between a liquid and a solid. For example, an exemplary surfactant can significantly lower the surface tension when present at very low concentrations (e.g., 5% w/w or less, such as 3% w/w or less, such as 1% w/w or less). Surfactants are amphiphilic, which means they are usually composed of both hydrophilic and hydrophobic or lipophilic groups, thus being capable of forming micelles or similar self-assembled structures in aqueous solutions. Known surfactants for pharmaceutical use include glycerol monooleat, benzethonium chloride, sodium docusate, phospholipids, polyethylene alkyl ethers, sodium lauryl sulfate and tricaprylin (anionic surfactants); benzalkonium chloride, citrimide, cetylpyridinium chloride and phospholipids (cationic surfactants); and alpha tocopherol, glycerol monooleate, myristyl alcohol, phospholipids, poloxamers, polyoxyethylene alkyl ethers, polyoxyethylene castor oil derivatives, polyoxyethylene sorbintan fatty acid esters, polyoxyethylene sterarates, polyoxyl 15 hydroxystearate, polyoxylglycerides, polysorbates, propylene glycol dilaurate, propylene glycol monolaurate, sorbitan esters sucrose palmitate, sucrose stearate, tricaprylin and TPGS (Nonionic and zwitterionic surfactants).

A “diluent” of interest herein is one which is pharmaceutically acceptable (safe and non-toxic for administration to a human) and is useful for the preparation of a reconstituted formulation. Exemplary diluents are liquids, preferably aqueous, and include sterile water, bacteriostatic water for injection (BWFI), a pH buffered solution (e.g. phosphate-buffered saline), sterile saline solution, Ringer's solution or dextrose solution.

### Specific Aspects and Embodiments of the Invention

In a first aspect the present invention provides an antibody binding to human AXL or an antibody-drug conjugate (ADC) comprising said antibody, for use in treating cancer in a subject, wherein
said cancer is resistant to or is predicted to be or become resistant to;
said cancer has failed to respond to, or is predicted to fail to respond to; and/or
said subject has relapsed after or is predicted to relapse after
   treatment with an inhibitor of the interaction between a programmed cell death-1 (PD-1) receptor and its ligand.

In the context of the invention the response to treatment with an inhibitor of the interaction between a programmed cell death-1 (PD-1) receptor and its ligand as well as whether a cancer is resistant to or has failed to respond to such treatment and whether a subject has relapsed after such treatment may be evaluated by a person of skill in the art according to known methods, e.g., the guidelines of the NCCN or ESMO. In a specific embodiment, the evaluation can be based on the following criteria (RECIST Criteria v1.1):

TABLE 1
Definition of Response (RECIST Criteria v1.1)

| | Category | Criteria |
|---|---|---|
| Based on | Complete Response | Disappearance of all target lesions. |
| target lesions | (CR) | Any pathological lymph nodes |
| | | must have reduction in short axis |
| | | to <10 mm. |
| | Partial Response | ≥30% decrease in the sum of the |
| | (PR) | LD of target lesions, taking as |
| | | reference the baseline sum LD. |
| | Stable Disease | Neither sufficient shrinkage to |
| | (SD) | qualify for PR nor sufficient |
| | | increase to qualify for PD, taking |
| | | as reference the smallest sum of |
| | | LDs since the treatment started. |
| | Progressive Disease | ≥20% increase in the sum of the |
| | (PD) | LDs of target lesions, taking as |
| | | reference the smallest sum of |
| | | the LDs recorded since the |
| | | treatment started or the |
| | | appearance of one or more |
| | | new lesions. |
| Based on non- | CR | Disappearance of all non-target |
| target lesions | | lesions and normalization of |
| | | tumor marker level. All lymph |
| | | nodes must be non-pathological |
| | | in size (<10 mm short axis). |
| | SD | Persistence of one or more |
| | | non-target lesion(s) or/and |
| | | maintenance of tumor marker |
| | | level above the normal limits. |
| | PD | Appearance of one or more new |
| | | lesions and/or unequivocal |
| | | progression of existing non- |
| | | target lesions. |

The same criteria may be applied when evaluating the effectiveness of the treatment with the antibody binding to human AXL or the ADC according to the present invention.

The said ligand PD-1 may in particular be programmed cell death-ligand 1 (PD-L1) or programmed cell death-ligand 2 (PD-L2).

The inhibitor may be selected from the group consisting of an antibody, such as a monoclonal antibody, that binds PD-1, an antibody, such as a monoclonal antibody, that binds PD-L1 and an antibody, such as a monoclonal antibody, that binds PD-L2.

The cancer may be a solid tumor, such as a metastatic, solid tumor, such as a metastatic, locally advanced tumor.

The antibody or ADC my be for use in treatment, wherein the cancer is a tumor selected from the group consisting of a melanoma, a carcinoma, a sarcoma (such as an undifferentiated pleomorphic sarcoma, aliposarcoma, a leiomyosarcoma, a synovial sarcoma, a Ewing's sarcoma, an osteosarcoma or a chondrosarcoma), an adenoma, a glioma, a hematologic tumor and a tumor of the lymphoid tissue.

Further, the antibody or ADC may be for use in treatment, wherein the solid tumor is selected from the group consisting of a melanoma, a carcinoma (such as squamous cell carcinoma of the head and neck (SCCHN)), a sarcoma (such as an undifferentiated pleomorphic sarcoma, aliposarcoma, a leiomyosarcoma, a synovial sarcoma, a Ewing's sarcoma, an osteosarcoma or a chondrosarcoma), an adenoma, and a glioma.

The solid tumor may in particular be selected from the group consisting of a carcinoma, a sarcoma (such as an undifferentiated pleomorphic sarcoma, aliposarcoma, a leiomyosarcoma, a synovial sarcoma, a Ewing's sarcoma, an osteosarcoma, a gastrointestinal stromal tumor (GIST), a rhabdomyosarcoma or a chondrosarcoma), an adenoma, and a glioma.

The cancer may be selected from from the group consisting of endometrial/cervical cancer, lung cancer (such as small cell lung cancer or non-small cell lung cancer), thyroid cancer, colon cancer, kidney cancer, renal cancer, ovary cancer, breast cancer (such as such as estrogen receptor alpha negative cancer, estrogen receptor alpha positive cancer or triple negative breast cancer; i.e. breast cancer tested negative for estrogen receptors (ER−), progesterone receptors (PR−), and human epidermal growth factor receptor 2 (HER2−)), esophagus cancer, skin cancer, melanoma (such as malignant melanoma), pancreatic cancer (such as unresectable advanced or metastatic pancreatic cancer), gastrointestinal stromal tumors (GISTs), and hematological cancer (such as leukemia; e.g. acute lymphoblastic leukemia, acute myeloid leukemia, chronic lymphocytic leukemia or chronic myeloid leukemia).

In particular, the cancer may be a metastasic, solid tumor other than a melanoma.

The antibody or ADC for use according to the invention, may be for use wherein said subject has documented progressive disease during or after last prior treatment with an inhibitor of the interaction between a programmed cell death-1 (PD-1) receptor and its ligand. Again, a person of skill in the art may evaluate whether a subject has documented progressive disease according to known methods; e.g. the guidelines of the NCCN or ESMO. The evaluation may in particular be based on the RECIST Criteria set forth in Table 1 above.

The antibody or ADC may in particular be used to treat subjects in which the resistance to, the failure to respond to or the relapse from the treatment with an inhibitor of the interaction between a programmed cell death-1 (PD-1) receptor and its ligand is associated with increased expression of AXL. In relation to the present invention, the inhibitor of the interaction between a programmed cell death-1 (PD-1) receptor and its ligand may be selected from the group consisting of Opdivo/Nivolumab (Bristol-Myers Squibb), Keytruda/pembrolizumab (Merck & Co), Amp-514/MEDI0680 (Amplimmune), BGB-A317 (BeiGene), REGN2810 (Regeneron), TSR-042 (Tesaro/AnaptysBio), CBT-501/genolimzumab (Genor Bio/CBT Pharma), PF-06801591 (Pfizer), JS-001 (Shanghai Junshi Bio), SHR-1210/INCSHR-1210 (Incyte corp), PDR001 (Novartis), BCD-100 (BioCad), AGEN2034 (Agenus), IBI-308 Innovent Biologics), BI-754091 (Boehringer Ingelheim).

Further, according to the invention, the inhibitor of the interaction between a programmed cell death-1 (PD-1) receptor and its ligand may be selected from the group consisting of Tecentriq/RG7446; MPDL-3280A, atezolizumab (Roche), Imfinzi/MEDI-4736/durvalumab (AstraZeneca), Bavencio/MSB-0010718C/avelumab (Merck Serono/Pfizer), KN-035-(3DMed/Alphamab C0), CX-072 (CytomX), LY-3300054 (Eli Lilly), MSB0011359C*/M-7824 (Merck KGaA), FAZ053 (Novartis), SHR-1316 (Atridia), ansd CA-170 (Aurigene/Curis).

The antibody binding to human AXL or said ADC may be provided to the subject as monotherapy.

Alternatively, the antibody binding to human AXL or said ADC may be provided to the subject as part of a combination therapy.

The ADC used according to the invention may comprise a therapeutic moiety, which is a cytotoxic agent, a chemotherapeutic drug or a radioisotope that is linked to the antibody, optionally with a linker.

In the ADC used according to the invention, the therapeutic moiety may be a cytotoxic agent, optionally linked to the ADC with a linker.

The cytotoxic agent may be linked to the antibody binding to human AXL with a cleavable linker, such as N-succinimydyl 4-(2-pyridyldithio)-pentanoate (SSP), maleimidocaproyl-valine-citrulline-p-aminobenzyloxycarbonyl (mc-vc-PAB) or AV-1 K-lock valine-citrulline.

In particular, the cytotoxic agent may be linked to the antibody binding to human AXL with a non-cleavable linker, such as succinimidyl-4(N-maleimidomethyl)cyclohexane-1-carboxylate (MCC) or maleimidocaproyl (MC).

Preferably, the linker has the formula -MC-vc-PAB-, wherein
a) MC is:

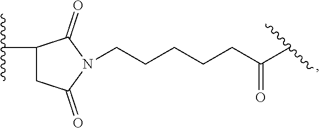 [SMILES: *C(=O)CCCCCN1C(=O)CC(*)C1=O]

b) vc is the dipeptide valine-citrulline, and
c) PAB is:

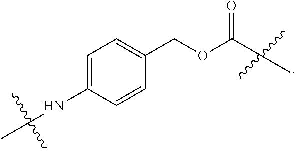 [SMILES: *Nc1ccc(COC(*)=O)cc1]

The cytotoxic agent may be selected from the group consisting of DNA-targeting agents, e.g. DNA alkylators and cross-linkers, such as calicheamicin, duocarmycin, rachelmycin (CC-1065), pyrrolo[2,1-c][1,4] benzodiazepines (PBDs), and indolinobenzodiazepine (IGN); microtubule-targeting agents, such as duostatin, such as duostatin-3, auristatin, such as monomethylauristatin E (MMAE) and monomethylauristatin F (MMAF), auristatin peptide analogs, dolastatin, maytansine, N(2′)-deacetyl-N(2′)-(3-marcapto-1-oxopropyl)-maytansine (DM1), and tubulysin, paclitaxel, docetaxel, vinblastine, vincristine, vinorelbine, maytansanoids, tubulysins; and nucleoside analogs; or analogs, derivatives, or prodrugs thereof.

The cytotoxic agent monomethyl auristatin E (MMAE) may be linked to the antibody via a valine-citrulline (VC) linker and the maleimidocaproyl (MC)linker, wherein the combination of the cytotoxic agent and the linkers has the chemical structure;

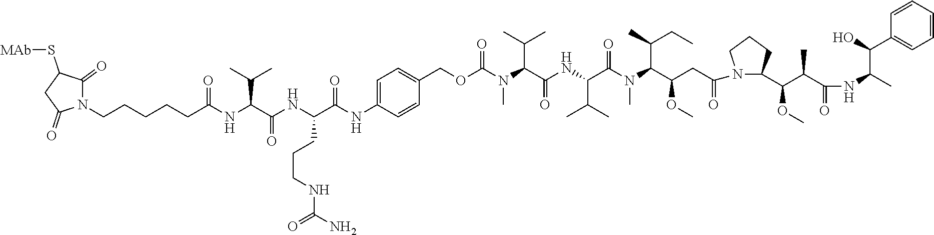

wherein MAb is the antibody.

In particular embodiments, the linker is attached to MMAE (vcMMAE), wherein vcMMAE is:

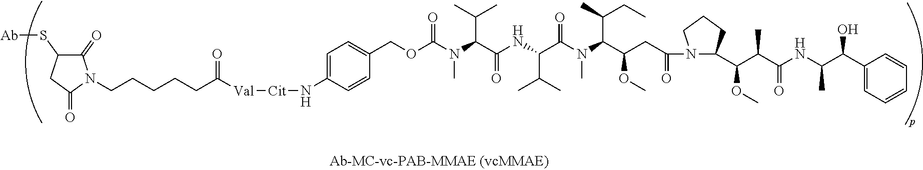

wherein p denotes a number form 1 to 8, S represents a sulfhydryl residue of the antibody, and Ab designates the antibody or antigen-binding fragments. In particular, p may be 1, 2, 3, 4, 5, 6, 7 or 8. Preferably, p is 4.

The average value of p in a population of the antibody-drug conjugate may in particular be about 1, such as 1; about 2, such as 2; about 3, such as 3; about 4, such as 4; about 5, such as 5; about 6, such as 6; about 7, such as 7 or about 8, such as 8. Preferably, the average value of p in a population of the antibody-drug conjugate is about 4, such as 4.

In particular, the cytotoxic agent may be monomethyl auristatin F (MMAF);

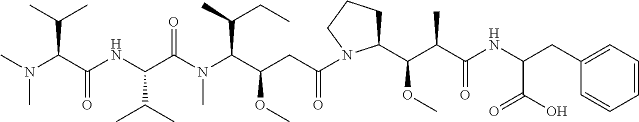 [SMILES: CC[C@H](C)[C@@H]([C@@H](CC(=O)N1CCC[C@H]1[C@H](OC)[C@@H](C)C(=O)NC(Cc1ccccc1)C(=O)O)OC)N(C)C(=O)[C@@H](NC(=O)[C@H](C(C)C)N(C)C)C(C)C]

wherein the antibody is linked to MMAF at the nitrogen (N) on the left-hand side of the chemical structure above by the appropriate linker.

In one embodiment, the cytotoxic agent monomethyl auristatin F (MMAF) is linked to the antibody via a maleimidocaproyl (mc)-linker, wherein the combination of the cytotoxic agent and linker has the chemical structure;

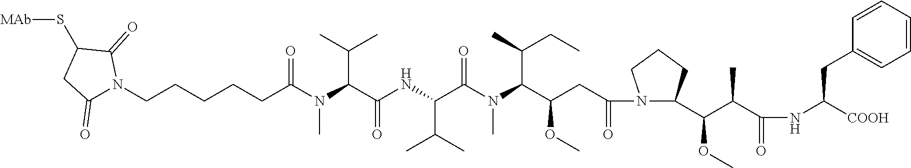

wherein MAb is the antibody.

In the ADC for use according to the invention may be an ADC, wherein
(a) the linker is cleavable and the cytotoxic agent has bystander kill capacity;
(b) the linker is cleavable and the cytotoxic agent does not have bystander kill capacity;
(c) the linker is non-cleavable and the cytotoxic agent has bystander kill capacity; or
(d) the linker is non-cleavable and the cytotoxic agent does not have bystander kill capacity.

In the context of the present invention, the term “bystander kill capacity” may be used interchangeably with “bystander killing effect”, “bystander kill”, or “bystander cytotoxicity”. The terms refer to the effect where the cytotoxic agent that is conjugated to the antibody by either a cleavable or non-cleavable linker has the capacity to diffuse across cell membranes after the release from the antibody and thereby cause killing of neighboring cells. When the cytotoxic agent is conjugated by a cleavable or non-cleavable linker, it may be either the cytotoxic agent only or the cytotoxic agent with a part of the linker that has the bystander kill capacity. The capacity to diffuse across cell membranes is related to the hydrophobicity of the cytotoxic agent or the combination of the cytotoxic agent and the linker. Such cytotoxic agents may advantageously be membrane-permeable toxins, such as MMAE that has been released from the antibody by proteases. Especially in tumors with heterogeneous target expression and in solid tumors where antibody penetration may be limited, a bystander killing effect may be desirable.

A cytotoxic agent that does not have “bystander kill capacity” does not have the capacity to diffuse across cell membranes after release from the antibody. Thus, such cytotoxic agents or combinations of the cytotoxic agent with the linker, will not be able to kill neighboring cells upon release from the antibody. It is believed without being bound by theory, that such combinations of a cytotoxic agent and either a cleavable or non-cleavable linker will only kill cells expressing the target that the antibody binds.

In particular, the linker may be mc-vc-PAB and the cytotoxic agent may be MMAE.

Alternatively, the linker may be SSP and the cytotoxic agent may be DM1.

The cytotoxic agent may in particular be duostatin-3.

With respect to the antibody or ADC for use as disclosed herein, it is preferred that the antibody binding to human AXL does not compete with Growth Arrest-Specific 6 (Gas6) for binding to human AXL.

Further, it is preferred that maximal antibody binding to human AXL in the presence of Gas6 is at least 90%, such as at least 95%, such as at least 97%, such as at least 99%, such as 100%, of binding in the absence of Gas6 as determined by a competition assay, wherein competition between said antibody binding to human AXL and said Gas6 is determined on A431 cells pre-incubated with Gas6 and without Gas6.

In the antibody or ADC for use as provided in the present application, the antibody binding to human AXL may in particular have a binding affinity (K_{D}) in the range of 0.3×10⁻⁹ to 63×10⁻⁹ M to human AXL, optionally wherein the binding affinity is measured using a Bio-layer Interferometry using soluble AXL extracellular domain.

The antibody binding to human AXL may have a dissociation rate of 9.7×10⁻⁵ to 4.4×10⁻³ s⁻¹ to AXL, optionally wherein the dissociation rate is measured by Bio-layer Interferometry using soluble recombinant AXL extracellular domain.

In relation to the antibody or ADC for use as provided in the present application, the amino acid sequence of the human AXL may be as specified in SEQ ID NO:130.

The antibody or ADC for use as provided in the present application may be an antibody or ADC, which binds to cynomolgus monkey AXL as specified in SEQ ID NO:147.

The antibody or ADC for use as provided in the present application, may be an antibody or ADC wherein the antibody binding to human AXL comprises at least one binding region comprising a VH region and a VL region selected from the group consisting of:
(a) a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107];
(b) a VH region comprising the CDR1, CDR2, and CDR3 sequences of SEQ ID Nos.: 46, 47, and 48, respectively; and a VL region comprising the CDR1, CDR2, and CDR3 sequences of SEQ ID Nos.: 49, AAS, and 50, respectively, [148];
(c) a VH region comprising the CDR1, CDR2, and CDR3 sequences of SEQ ID Nos.: 114, 115, and 116, respectively, and a VL region comprising the CDR1, CDR2, and CDR3 sequences of SEQ ID Nos.: 117, DAS, and 118, respectively [733];
(d) a VH region comprising the CDR1, CDR2, and CDR3 sequences of SEQ ID Nos.: 51, 52, and 53, respectively; and a VL region comprising the CDR1, CDR2, and CDR3 sequences of SEQ ID Nos.: 55, GAS, and 56, respectively [154];
(e) a VH region comprising the CDR1, CDR2, and CDR3 sequences of SEQ ID Nos.: 51, 52, and 54, respectively; and a VL region comprising the CDR1, CDR2, and CDR3 sequences of SEQ ID Nos.: 55, GAS, and 56, respectively [154-M103L];
(f) a VH region comprising the CDR1, CDR2, and CDR3 sequences of SEQ ID Nos.: 57, 58, and 59, respectively; and a VL region comprising the CDR1, CDR2, and CDR3 sequences of SEQ ID Nos.: 60, GAS, and 61, respectively, [171];
(g) a VH region comprising the CDR1, CDR2, and CDR3 sequences of SEQ ID Nos.: 62, 63, and 64, respectively; and a VL region comprising the CDR1, CDR2, and CDR3 sequences of SEQ ID Nos.: 65, GAS, and 66, respectively, [172];
(h) a VH region comprising the CDR1, CDR2, and CDR3 sequences of SEQ ID Nos.: 67, 68, and 69, respectively; and a VL region comprising the CDR1, CDR2, and CDR3 sequences of SEQ ID Nos.: 70, GAS, and 71, respectively, [181];
(i) a VH region comprising the CDR1, CDR2, and CDR3 sequences of SEQ ID Nos.: 72, 73, and 75, respectively; and a VL region comprising the CDR1, CDR2, and CDR3 sequences of SEQ ID Nos.: 76, ATS, and 77, respectively, [183];
(j) a VH region comprising the CDR1, CDR2, and CDR3 sequences of SEQ ID Nos.: 72, 74, and 75, respectively; and a VL region comprising the CDR1, CDR2, and CDR3 sequences of SEQ ID Nos.: 76, ATS, and 77, respectively, [183-N52Q];
(k) a VH region comprising the CDR1, CDR2, and CDR3 sequences of SEQ ID Nos.: 78, 79, and 80, respectively; and a VL region comprising the CDR1, CDR2, and CDR3 sequences of SEQ ID Nos.: 81, AAS, and 82, respectively, [187];
(l) a VH region comprising the CDR1, CDR2, and CDR3 sequences of SEQ ID Nos.: 83, 84, and 85, respectively; and a VL region comprising the CDR1, CDR2, and CDR3 sequences of SEQ ID Nos.: 86, GAS, and 87, respectively, [608-01];
(m) a VH region comprising the CDR1, CDR2, and CDR3 sequences of SEQ ID Nos.: 88, 89, and 90, respectively; and a VL region comprising the CDR1, CDR2, and CDR3 sequences of SEQ ID Nos.: 91, GAS, and 92, respectively, [610-01];
(n) a VH region comprising the CDR1, CDR2, and CDR3 sequences of SEQ ID Nos.: 93, 94, and 95, respectively; and a VL region comprising the CDR1, CDR2, and CDR3 sequences of SEQ ID Nos.: 96, GAS, and 97, respectively, [613];
(o) a VH region comprising the CDR1, CDR2, and CDR3 sequences of SEQ ID Nos.: 98, 99, and 100, respectively; and a VL region comprising the CDR1, CDR2, and CDR3 sequences of SEQ ID Nos.: 101, DAS, and 102, respectively, [613-08];
(p) a VH region comprising the CDR1, CDR2, and CDR3 sequences of SEQ ID Nos.: 103, 104, and 105, respectively; and a VL region comprising the CDR1, CDR2, and CDR3 sequences of SEQ ID Nos.: 106, GAS, and 107, respectively, [620-06];
(q) a VH region comprising the CDR1, CDR2, and CDR3 sequences of SEQ ID Nos.: 108, 109, and 110, respectively; and a VL region comprising the CDR1, CDR2, and CDR3 sequences of SEQ ID Nos.: 112, AAS, and 113, respectively, [726];
(r) a VH region comprising the CDR1, CDR2, and CDR3 sequences of SEQ ID Nos.: 108, 109, and 111, respectively; and a VL region comprising the CDR1, CDR2, and CDR3 sequences of SEQ ID Nos.: 112, AAS, and 113, respectively, [726-M101L];
(s) a VH region comprising the CDR1, CDR2, and CDR3 sequences of SEQ ID Nos.: 41, 42, and 43, respectively; and a VL region comprising the CDR1, CDR2, and CDR3 sequences of SEQ ID Nos.: 44, AAS, and 45, respectively, [140];
(t) a VH region comprising the CDR1, CDR2, and CDR3 sequences of SEQ ID Nos.: 93, 94, and 95, respectively, and a VL region comprising the CDR1, CDR2, and CDR3 sequences of SEQ ID Nos.: 128, XAS, wherein X is D or G, and 129, respectively, [613/613-08];
(u) a VH region comprising the CDR1, CDR2, and CDR3 sequences of SEQ ID Nos.: 46, 119, and 120, respectively; and a VL region comprising CDR1, CDR2, and CDR3 sequences of SEQ ID Nos.: 49, AAS, and 50, respectively, [148/140];
(v) a VH region comprising the CDR1, CDR2, and CDR3 sequences of SEQ ID Nos.: 123, 124, and 125, respectively; and a VL region comprising CDR1, CDR2, and CDR3 sequences of SEQ ID Nos.: 60, GAS, and 61, respectively [171/172/181]; and
(w) a VH region comprising the CDR1, CDR2, and CDR3 sequences of SEQ ID Nos.: 121, 109, and 122, respectively; and a VL region comprising the CDR1, CDR2, and CDR3 sequences of SEQ ID Nos.: 112, AAS, and 113, respectively [726/187]; and
(x) a VH region comprising the CDR1, CDR2, and CDR3 sequences of SEQ ID Nos.:93, 126, and 127, respectively; and a VL region comprising the CDR1, CDR2, and CDR3 sequences of SEQ ID Nos.: 96, GAS, and 97, respectively [613/608-01/610-01/620-06].

In particular, the antibody or ADC for the use as provided herein may be an antibody or ADC, wherein the antibody binding to human AXL comprises at least one binding region comprising
(a) a VH region comprising the CDR1, CDR2, and CDR3 sequences of SEQ ID Nos.: 36, 37, and 38, respectively, and
(b) a VL region comprising the CDR1, CDR2, and CDR3 sequences of SEQ ID Nos.: 39, GAS, and 40, respectively [107].

Also, the antibody or ADC for the use as provided in the present application may be an antibody or ADC, wherein the antibody binding to human AXL comprises at least one binding region comprising a VH region and a VL region selected from the group consisting of:
(a) a VH region at least 90%, such as at least 95%, such as at least 97%, such as at least 99% identical to SEQ ID No: 1 and a VL region at least 90%, such as at least 95%, such as at least 97%, such as at least 99% identical to SEQ ID No: 2 [107];
(b) a VH region at least 90%, such as at least 95%, such as at least 97%, such as at least 99% identical to SEQ ID No: 5 and a VL region at least 90%, such as at least 95%, such as at least 97%, such as at least 99% identical to SEQ ID No: 6 [148];
(c) a VH region at least 90%, such as at least 95%, such as at least 97%, such as at least 99% identical to SEQ ID No: 34 and a VL region at least 90%, such as at least 95%, such as at least 97%, such as at least 99% identical to SEQ ID No: 35 [733]
(d) a VH region at least 90%, such as at least 95%, such as at least 97%, such as at least 99% identical to SEQ ID No: 7 and a VL region at least 90%, such as at least 95%, such as at least 97%, such as at least 99% identical to SEQ ID No: 9 [154];
(e) a VH region at least 90%, such as at least 95%, such as at least 97%, such as at least 99% identical to SEQ ID No: 10 and a VL region at least 90%, such as at least 95%, such as at least 97%, such as at least 99% identical to SEQ ID No: 11 [171];
(f) a VH region at least 90%, such as at least 95%, such as at least 97%, such as at least 99% identical to SEQ ID No: 16 and a VL region at least 90%, such as at least 95%, such as at least 97%, such as at least 99% identical to SEQ ID No: 18 [183];
(g) a VH region at least 90%, such as at least 95%, such as at least 97%, such as at least 99% identical to SEQ ID No: 25 and a VL region at least 90%, such as at least 95%, such as at least 97%, such as at least 99% identical to SEQ ID No: 26 [613];
(h) a VH region at least 90%, such as at least 95%, such as at least 97%, such as at least 99% identical to SEQ ID No: 31 and a VL region at least 90%, such as at least 95%, such as at least 97%, such as at least 99% identical to SEQ ID No: 33 [726];
(i) a VH region at least 90%, such as at least 95%, such as at least 97%, such as at least 99% identical to SEQ ID No: 3 and a VL region at least 90%, such as at least 95%, such as at least 97%, such as at least 99% identical to SEQ ID No: 4 [140];
(j) a VH region at least 90%, such as at least 95%, such as at least 97%, such as at least 99% identical to SEQ ID No:8 and a VL region at least 90%, such as at least 95%, such as at least 97%, such as at least 99% identical to SEQ ID No:9 [154-M103L];
(k) a VH region at least 90%, such as at least 95%, such as at least 97%, such as at least 99% identical to SEQ ID No:12 and a VL region at least 90%, such as at least 95%, such as at least 97%, such as at least 99% identical to SEQ ID No:13 [172];
(l) a VH region at least 90%, such as at least 95%, such as at least 97%, such as at least 99% identical to SEQ ID No:14 and a VL region at least 90%, such as at least 95%, such as at least 97%, such as at least 99% identical to SEQ ID No:15 [181];
(m) a VH region at least 90%, such as at least 95%, such as at least 97%, such as at least 99% identical to SEQ ID No:17 and a VL region at least 90%, such as at least 95%, such as at least 97%, such as at least 99% identical to SEQ ID No:18 [183-N52Q];
(n) a VH region at least 90%, such as at least 95%, such as at least 97%, such as at least 99% identical to SEQ ID No:19 and a VL region at least 90%, such as at least 95%, such as at least 97%, such as at least 99% identical to SEQ ID No:20 [187];
(o) a VH region at least 90%, such as at least 95%, such as at least 97%, such as at least 99% identical to SEQ ID No:21 and a VL region at least 90%, such as at least 95%, such as at least 97%, such as at least 99% identical to SEQ ID No:22 [608-01];
(p) a VH region at least 90%, such as at least 95%, such as at least 97%, such as at least 99% identical to SEQ ID No:23 and a VL region at least 90%, such as at least 95%, such as at least 97%, such as at least 99% identical to SEQ ID No:24 [610-01];
(q) a VH region at least 90%, such as at least 95%, such as at least 97%, such as at least 99% identical to SEQ ID No:27 and a VL region at least 90%, such as at least 95%, such as at least 97%, such as at least 99% identical to SEQ ID No:28 [613-08];
(r) a VH region at least 90%, such as at least 95%, such as at least 97%, such as at least 99% identical to SEQ ID No:29 and a VL region at least 90%, such as at least 95%, such as at least 97%, such as at least 99% identical to SEQ ID No:30 [620-06]; and
(s) a VH region at least 90%, such as at least 95%, such as at least 97%, such as at least 99% identical to SEQ ID No:32 and a VL region at least 90%, such as at least 95%, such as at least 97%, such as at least 99% identical to SEQ ID No:33 [726-M101L].

Further, the antibody or ADC for use as disclosed in the present application may be an antibody or ADC, wherein the at least one binding region of the antibody comprises a VH region and a VL region selected from the group consisting of;
(a) a VH region comprising SEQ ID No: 1 and a VL region comprising SEQ ID No: 2 [107];
(b) a VH region comprising SEQ ID No: 5 and a VL region comprising SEQ ID No: 6 [148];
(c) a VH region comprising SEQ ID No: 34 and a VL region comprising SEQ ID No: 35 [733]
(d) a VH region comprising SEQ ID No: 7 and a VL region comprising SEQ ID No: 9 [154];
(e) a VH region comprising SEQ ID No: 10 and a VL region comprising SEQ ID No: 11 [171];
(f) a VH region comprising SEQ ID No: 16 and a VL region comprising SEQ ID No: 18 [183];
(g) a VH region comprising SEQ ID No: 25 and a VL region comprising SEQ ID No: 26 [613];
(h) a VH region comprising SEQ ID No: 31 and a VL region comprising SEQ ID No: 33 [726];
(i) a VH region comprising SEQ ID No: 3 and a VL region comprising SEQ ID No: 4 [140];
(j) a VH region comprising SEQ ID No:8 and a VL region comprising SEQ ID No:9 [154-M103L];
(k) a VH region comprising SEQ ID No:12 and a VL region comprising SEQ ID No:13 [172];
(l) a VH region comprising SEQ ID No:14 and a VL region comprising SEQ ID No:15 [181];
(m) a VH region comprising SEQ ID No:17 and a VL region comprising SEQ ID No:18 [183-N52Q];
(n) a VH region comprising SEQ ID No:19 and a VL region comprising SEQ ID No:20 [187];
(o) a VH region comprising SEQ ID No:21 and a VL region comprising SEQ ID No:22 [608-01];
(p) a VH region comprising SEQ ID No:23 and a VL region comprising SEQ ID No:24 [610-01];
(q) a VH region comprising SEQ ID No:27 and a VL region comprising SEQ ID No:28 [613-08];
(r) a VH region comprising SEQ ID No:29 and a VL region comprising SEQ ID No:30 [620-06]; and
(s) a VH region comprising SEQ ID No:32 and a VL region comprising SEQ ID No:33 [726-M101L].

In the antibody or ADC for use as provided in the present application, the at least one binding region of the antibody binding to human AXL may in particular comprise a VH region comprising SEQ ID No: 1 and a VL region comprising SEQ ID No: 2 [107].

In the antibody or ADC for use as provided in the present application, the antibody binding to human AXL may comprise at least one binding region comprising a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107].

In the antibody or ADC for use as provided in the present application, the antibody may bind to an epitope on AXL wherein the epitope is recognized by any of the antibodies defined above; in particular an antibody having a VH region as defined above.

In the antibody or ADC for use as provided in the present application, the antibody binding to human AXL may in particular bind to an epitope within the Ig1 domain, or Ig1-like domain, of AXL, the epitope comprising or requiring one or more amino acids corresponding to positions L121 to Q129 or T112 to Q124 of human AXL.

In the antibody or ADC for use as provided in the present application, the antibody binding to human AXL may bind to an epitope within the Ig2 domain or Ig2-like domain, of AXL, wherein the epitope comprising or requiring the amino acids corresponding to position D170 or the combination of D179 and one or more amino acids corresponding to positions T182 to R190 of human AXL.

In the antibody or ADC for use as provided in the present application, the antibody may bind to an epitope within the FN1 domain, or FN-like domain, of human AXL, wherein the epitope comprises or requires one or more amino acids corresponding to positions Q272 to A287 and G297 to P301 of human AXL.

In the antibody or ADC for use as provided in the present application, the antibody binding to human AXL may bind to an epitope within the FN2 domain of human AXL, wherein the epitope comprises or requires the amino acids corresponding to positions A359, R386, and one or more amino acids corresponding to positions Q436 to K439 of human AXL.

In relation to the antibody or ADC for the use as provided herein, the ACD may be one that is able to induce tumor regression in an SKMel-147 human xenograft mouse model and/or in a BLM melanoma xenograft model.

The SKMel-147 human xenograft mouse model and/or the BLM melanoma xenograft model is/are preferably resistant to anti-PD-1 treatment, such as treatment with an inhibitor of the interaction between a programmed cell death-1 (PD-1) receptor and its ligand.

The SKMel-14 human xenograft mouse model may be generated at described in Example 5 herein or essentially as described in Example 5 herein.

The BLM melanoma xenograft model may be generated as described in Example 6 herein or essentially as described in Example 6 herein.

In the antibody or ADC for use as provided in the present application, the antibody binding to human AXL may comprise a heavy chain of an isotype selected from the group consisting of IgG1, IgG2, IgG3, and IgG4.

In the antibody or ADC for use as provided in the present application, the isotype of the antibody binding to human AXL may in particular be IgG1, such as human IgG1, optionally allotype IgG1m(f).

In the antibody or ADC for use as provided in the present application, he antibody binding to human AXL may be a monoclonal antibody or an antigen-binding fragment thereof, such as a full-length monoclonal antibody, such as a full-length monoclonal IgG1,κ antibody.

The antibody is preferably a humanized or human antibody.

In currently preferred embodiments, the antibody is Enapotamab.

In equally preferred embodiments, the ADC is Enapotamab vedotin.

In the antibody or ADC for use as provided in the present application, the antibody binding to human AXL may be an effector-function-deficient antibody, a stabilized IgG4 antibody or a monovalent antibody.

In the antibody or ADC for use as provided in the present application, the heavy chain of the antibody binding to human AXL may have been modified such that the entire hinge region has been deleted.

In the antibody or ADC for use as provided in the present application, the sequence of the antibody binding to human AXL may have been modified so that it does not comprise any acceptor sites for N-linked glycosylation.

In the antibody or ADC for use as provided in the present application, the antibody binding to human AXL may be a single-chain antibody.

In the antibody or ADC for use as provided in the present application, the antibody binding to human AXL may be a bispecific antibody comprising a first binding region of an antibody according to any one of the preceding claims, and a second binding region which binds a different target or epitope than the first binding region.

In the antibody or ADC for use as provided in the present application, the bispecific antibody binding to human AXL may comprise a first and a second heavy chain, each of the first and second heavy chain comprises at least a hinge region, a CH2 and CH3 region, wherein in the first heavy chain at least one of the amino acids in the positions corresponding to positions selected from the group consisting of K409, T366, L368, K370, D399, F405, and Y407 in a human IgG1 heavy chain has been substituted, and in the second heavy chain at least one of the amino acids in the positions corresponding to a position selected from the group consisting of F405, T366, L368, K370, D399, Y407, and K409 in a human IgG1 heavy chain has been substituted, and wherein the substitutions of the first and the second heavy chains are not in the same positions.

In the antibody or ADC for use as provided in the present application, the amino acid in the position corresponding to K409 in a human IgG1 heavy chain may be R in the first heavy chain, and the amino acid in the position corresponding to F405 in a human IgG1 heavy chain may be L in the second heavy chain, or vise versa.

The antibody or ADC for use as set forth above may be in a formulation such as a formulation comprising one or more pharmaceutically acceptable excipients, carriers, stabilizers, bulking agents, surfactants and/or diluents, such as a pharmaceutical formulation.

The antibody or ADC for use as set forth above may in particular be in a lyophilized formulation.

The lyophilized formulation may be obtainable or may be obtained by lyophilizing an aqueous formulation comprising the antibody or ADC and one or more excipients, wherein the aqueous formulation is free of any surfactant.

The lyophilized formulation may in particular be one which is obtainable or is obtained by lyophilizing an aqueous formulation comprising the antibody or ADC and
a. a buffer providing for a pH of between about 5 and about 7 in the aqueous formulation;
b. at least one bulking agent; and
c. at least one non-reducing sugar which forms an amorphous phase with the antibody or ADC in solid state.

The aqueous formulation my be one which is free of any surfactant.

The aqueous formulation may comprise a buffer selected from the group consisting of histidine, citrate, 2-(N-morpholino)ethanesulfonic acid (MES), succinate, glycolate, carbonic acid and phosphate, or a combination of any thereof, wherein the pH of the aqueous formulation is in a range from about 5 to about 7, such as in a range from 5 to 7.

The aqueous formulation may in particular comprise a histidine buffer.

The aqueous formulation may comprise a buffer at a concentration of about 5 mM to about 100 mM, such as a concentration of 5 mM to 100 mM, such as from about 10 mM to about 50 mM buffer, such as from 10 mM to 50 mM buffer, such as from about 20 mM to about 40 mM, such as from 20 mM to 40 mM, such as from about 28 mM to about 32 mM, such as from 28 mM to 32 mM, such as about 30 mM buffer, such as 30 mM buffer.

The lyophilized formulation may comprise a bulking agent selected from mannitol, glycine, and a combination thereof.

The lyophilized formulation may in particular be one, which comprises mannitol.

The aqueous formulation may comprise a bulking agent at a concentration of about 1% (w/v) to about 5% (w/v), such as 1% (w/v) to 5% (w/v), such as about 2% (w/v) to about 4% (w/v), such as 2% (w/v) to 4% (w/v), such as from about 2.5% (w/v) to about 3.5% (w/v), such as from 2.5% (w/v) to 3.5% (w/v), such as about 3% (w/v), such as 3% (w/v).

The aqueous formulation may comprise a bulking agent at a concentration of about 50 mM to about 300 mM, such as a concentration of 50 mM to 300 mM, such as from about 100 mM to about 225 mM, such as from 100 mM to 225 mM, such as from about 150 mM to about 180 mM, such as from 150 mM to 180 mM, such as about 165 mM, such as 165 mM.

The lyophilized formulation may comprise a non-reducing sugar selected from sucrose, trehalose, and a combination thereof.

The lyophilized formulation may in particular be one that comprises sucrose.

The aqueous formulation may comprise a non-reducing sugar at a concentration of about 0.5% (w/v) to about 7% (w/v), such as a concentration of 0.5% (w/v) to 7% (w/v), such as from about 0.5% (w/v) to about 4% (w/v), such as from 0.5% (w/v) to 4% (w/v), such as from about 1% (w/v) to about 3% (w/v), such as from 1% (w/v) to 3% (w/v), or from about 2.5% to about 3.5%, or from 2.5% to 3.5%, such as about 3% (w/v), such as 3% (w/v).

The aqueous formulation may comprise a non-reducing sugar at a concentration of about 15 mM to about 200 mM, such as at a concentration of 15 mM to 200 mM, such as from about 30 mM to about 150 mM, such as from 30 mM to 150 mM, such as about 80 mM to about 100 mM, such as 80 mM to 100 mM, such as from about 70 to about 90 mM, such as from 70 to 90 mM, such as from about 84 mM to about 92 mM sucrose, such as from 84 mM to 92 mM sucrose, such as about 88 mM, such as 88 mM.

The lyophilized formulation may be one, which is obtainable or is obtained by lyophilizing an aqueous formulation, wherein the antibody or ADC concentration in the aqueous formulation is from about 5 mg/mL to about 30 mg/mL, from 5 mg/mL to 30 mg/mL, such as from about 7 mg/mL to about 20 mg/mL, such as from 7 mg/mL to 20 mg/mL, such as from about 8 mg/mL to about 15 mg/mL, such as from 8 mg/mL to 15 mg/mL, such as from about 9 mg/mL to about 11 mg/mL, such as from 9 mg/mL to 11 mg/mL such as about 10 mg/mL, such as 10 mg/mL.

The lyophilized formulation may be obtainable or be obtained by lyophilizing an aqueous formulation in which the pH is in a range from about 5.5 to 6.5, such as in a range from about 5.5 to 6.5, such as about 6, such as 6.

The lyophilized formulation may be a formulation, which is obtainable or is obtained by lyophilizing an aqueous formulation having a pH of about 5 to about 7, such as a pH of 5 to 7, and comprises
a. from about 5 mg/mL to about 30 mg/mL, such as from 5 mg/mL to 30 mg/mL of the antibody or ADC;
b. from about 10 mM to about 50 mM histidine, such as from 10 mM to 50 mM histidine;
c. from about 30 mM to about 150 mM sucrose or trehalose, such as from 30 mM to 150 mM sucrose or trehalose; and
d. from about 150 mM to about 180 mM mannitol or glycine, such as from 150 mM to 180 mM mannitol or glycine.

The aqueous formulation may have a pH in the range of about 5.5 to about 6.5, such as in the range of 5.5 to 6.5 and comprise
a. from about 9 mg/mL to about 11 mg/mL of the antibody or ADC, such as from 9 mg/mL to 11 mg/mL, of the antibody or ADC, such as about 10 mg/mL of the antibody or ADC, such as 10 mg/mL of the antibody or ADC;
b. from about 20 mM to about 40 mM histidine, such as from 20 mM to 40 mM histidine, such as about 30 mM histidine, such as 30 mM histidine;
c. from about 80 mM to about 100 mM sucrose, such as from 80 mM to 100 mM sucrose, such as about 88 mM sucrose, such as 88 mM sucrose; and
d. from about 150 mM to about 180 mM mannitol, such as from 150 mM to 180 mM mannitol, such as about 165 mM mannitol, such as 165 mM mannitol;
   wherein the aqueous formulation is free of any surfactant.

The antibody or ADC in said lyophilized formulation is is preferably stable at 2-8° C., such as at 5° C. for pharmaceutical use for at least 6 months, such as for at least 9 months, such as for at least 15 months or preferably for at least 18 months, or even more preferred for at least 24 months, or most preferred for at least 36 months.

The lyophilized formulation may be considered stable when it has less than 10% aggregates, such as less than 5.0% aggregates, such as less than 3.0% aggregates, such as less than 2.0% aggregates when stored at 5° C. for at least 6 months, such as for at least 9 months, such as for at least 15 months or preferably for at least 18 months, or even more preferred for at least 24 months, or most preferred for at least 36 months.

The stability is preferably determined by size-exclusion analysis, cIEF, or both.

Preferably, the lyophilized formulation contains less than 3.0% moisture, such as less than 2.0% moisture, such as less than 1% moisture, or less than 0.5% moisture.

The lyophilized formulation may be a formulation, which is free of any inorganic salts.

The pharmaceutical formulation may be obtained or may be obtainable by reconstituting the lyophilized formulation as defined above in a sterile aqueous diluent.

The pharmaceutical formulation may be a formulation, which has a pH of about 5 to about 7, such as a pH of about 5 to about 7, and comprise, in aqueous solution:
a. from about 5 mg/mL to about 30 mg/mL of the antibody or ADC, such as from 5 mg/mL to 30 mg/mL of the antibody or ADC;
b. from about 10 mM to about 50 mM histidine, such as from 10 mM to 50 mM histidine;
c. from about 30 mM to about 150 mM sucrose or trehalose, such as from 30 mM to 150 mM sucrose or trehalose; and
d. from about 50 mM to about 300 mM mannitol or glycine, such as from 50 mM to 300 mM mannitol or glycine.

The pharmaceutical formulation may have a pH in the range of about 5.5 to about 6.5 such as in the range of 5.5 to 6.5, and comprises:
a. from about 9 mg/mL to about 11 mg/mL of the antibody or ADC, such as from 9 mg/mL to 11 mg/mL of the antibody or ADC, such as about 10 mg/mL of the antibody or ADC, such as 10 mg/mL of the antibody or ADC;
b. from about 20 mM to about 40 mM histidine, such as from 20 mM to 40 mM histidine, such as about 30 mM histidine, such as 30 mM histidine;
c. from about 80 mM to about 100 mM sucrose, such as from 80 mM to 100 mM sucrose, such as about 88 mM sucrose, such as 88 mM sucrose; and
d. from about 150 mM to about 180 mM mannitol, such as from 150 mM to 180 mM mannitol, such as about 165 mM, such as 165 mM;
   wherein the aqueous formulation is free of any surfactant.

The antibody or ADC for use as set forth above may be in an aqueous formulation comprising one or more pharmaceutically acceptable excipients, wherein the aqueous formulation is free of any surfactant.

The antibody or ADC for use as set forth above may be in an aqueous formulation comprising a buffer and at least one stabilizer, wherein the pH of the aqueous formulation is between about 5 and about 7, such as between 5 and 7, and wherein the aqueous formulation is free of any surfactant.

The antibody or ADC for use as set forth above may be in an aqueous formulation comprising a buffer selected from the group consisting of histidine, citrate, MES, phosphate, carbonic acid, succinate, glycolate, or a combination of any thereof, wherein the pH of the aqueous formulation is in a range from about 5 to about 7, such as from 5 to 7.

The antibody or ADC for use as set forth above may in particular be in an aqueous formulation, comprising a histidine buffer.

The antibody or ADC for use as set forth above may be in an aqueous formulation comprising a buffer at a concentration of about 10 mM to about 50 mM, such as 10 mM to 50 mM, such as from about 20 mM to about 40 mM buffer, such as from 20 mM to 40 mM buffer, such as from about 28 mM to about 34 mM, such as from 28 mM to 34 mM, such as from about 29 mM to about 31 mM, such as from 29 mM to 31 mM, such as about 30 mM, such as 30 mM.

The antibody or ADC for use as set forth above may be in an aqueous formulation, comprising a stabilizer selected from the group consisting of mannitol, sucrose and trehalose.

The antibody or ADC for use as set forth above may be in an aqueous formulation, comprising a stabilizer which is mannitol.

The antibody or ADC for use as set forth above may be in an aqueous formulation, comprising a stabilizer at a concentration of about 20 mM to about 200 mM, such as of 20 mM to 200 mM, such as from about 30 mM to about 100 mM, such as from 30 mM to 100 mM, such as from about 40 mM to about 80 mM, such as from 40 mM to 80 mM, such as about 50 mM to about 60 mM, such as 50 mM to 60 mM, such as about 55 mM, such as 55 mM.

The antibody or ADC for use as set forth above may be in an aqueous formulation comprising a stabilizer selected from sucrose, trehalose and a combination thereof.

The antibody or ADC for use as set forth above may be in an aqueous formulation, which is free of any one or more of arginine, glycine, glutamic acid, sorbitol, trehalose, sucrose and sodium chloride.

The antibody or ADC for use as set forth above may be in an aqueous formulation, wherein the antibody or ADC concentration is from about 5 mg/mL to about 40 mg/mL, such as from 5 mg/mL to 40 mg/mL, such as from about 8 mg/mL to about 35 mg/mL, such as from 8 mg/mL to 35 mg/mL, such as from about 10 mg/mL to about 30 mg/mL, such as from 10 mg/mL to 30 mg/mL, such as from about 15 mg/mL to about 25 mg/mL, such as from 15 mg/mL to 25 mg/mL, such as about 20 mg/mL, such as 20 mg/mL.

The antibody or ADC for use as provided in the present application may be in an aqueous formulation, wherein the pH of the aqueous formulation is in a range from about 5.5 to 6.5, such as from about 5.5 to 6.5, such as about 6, such as 6.

The antibody or ADC for use as set forth above may be in an aqueous formulation having a pH of about 5 to about 7 and comprising
a. from about 5 mg/mL to about 40 mg/mL of the antibody or ADC, such as from 5 mg/mL to 40 mg/mL of the antibody or ADC, and
b. from about 10 mM to about 50 mM histidine, from 10 mM to 50 mM histidine;
c. from about 50 mM to about 300 mM mannitol, such as from 50 mM to 300 mM mannitol.

The antibody or ADC for use as provided above may be in an aqueous formulation, which has a pH in the range of about 5.5 to about 6.5, such as in the range of 5.5 to 6.5 and comprises
a. from about 15 mg/mL to about 25 mg/mL of the antibody or ADC, from 15 mg/mL to 25 mg/mL of the antibody or ADC, such as about 20 mg/mL of the antibody or ADC, such as 20 mg/mL of the antibody or ADC;
b. from about 20 mM to about 40 mM histidine, from 20 mM to 40 mM histidine such as about 30 mM histidine;
c. from about 50 mM to about 60 mM mannitol, from 50 mM to 60 mM mannitol, such as about 55 mM, such as 55 mM;
wherein the aqueous formulation is free of any added surfactant, amino acid excipient, NaCl, or a combination of any thereof.

The antibody or ADC for use as provided in the present application may be in a frozen aqueous formulation, which is obtained or is obtainable by freezing the aqueous formulation defined herein above.

The antibody or ADC for use as set forth above may be administered to said subject in therapeutically effective amounts and frequencies, such as
In at least one cycle comprising administration once every three weeks, such as on day 1 of a cycle of 21 days; or
in at least one cycle comprising administration once a week for three consecutive weeks followed by a one-week resting period without any administration of ADC so that each cycle time is 28 days including the resting period, such as on days 1, 8 and 15 in the cycle of 28 days.

As used herein, the term “resting period” is to be understood as a period of time wherein the antibody or ADC is administered at a substantially lower dose than that administered the preceding week, or wherein the antibody or ADC is not administered at all, e.g., during which the antibody or ADC is not administered at all. In a preferred embodiment of any aspect or embodiment herein, no antibody or ADC is administered during the resting period, in which case the resting period may alternatively be referred to as an “off-period”. A resting period or off-period of one week can also be referred to as a “resting week” or “off-week”, respectively

When provided for use as defined in the present application, the dose of the antibody or ADC in said cycle of 21 days may in particular be between 0.6 mg/kg and 4.0 mg/kg of the subject's body weight, such as between 0.6 mg/kg and 3.2 mg/kg of the subject's body weight, such as at a dose of about 0.6 mg/kg, such as at a dose of 0.6 mg/kg, or at a dose of about 0.8 mg/kg, such as at a dose of 0.8 mg/kg or at a dose of about 1.0 mg/kg, such as at a dose of 1.0 mg/kg, or at a dose of about 1.2 mg/kg, at a dose of 1.2 mg/kg, or at a dose of about 1.4 mg/kg, such as at a dose of 1.4 mg/kg, or at a dose of about 1.6 mg/kg, such as at a dose of 1.6 mg/kg, or at a dose of about 1.8 mg/kg, such as at a dose of 1.8 mg/kg, or at a dose of about 2.0 mg/kg, such at a dose of 2.0 mg/kg, or at a dose of about 2.2 mg/kg, such as at a dose of 2.2 mg/kg, or at a dose of about 2.4 mg/kg, such as at a dose of 2.4 mg/kg, or at a dose of about 2.6 mg/kg, such as at a dose of 2.6 mg/kg, or at a dose of about 2.8 mg/kg, such as at a dose of 2.8 mg/kg, or at a dose of about 3.0 mg/kg, such as at a dose of about 3.0 mg/kg, or at a dose of about 3.2 mg/kg, such as at a dose of 3.2 mg/kg.

When provided for use as defined in the present application, the dose of the antibody or ADC in said cycle of 28 days may be between 0.45 mg/kg and 2.0 mg/kg of the subject's body weight, such as between 0.45 mg/kg and 2.0 mg/kg of the subject's body weight, such as at a dose of about 0.45 mg/kg, such as at a dose of 0.45 mg/kg, or at a dose of about 0.5 mg/kg, such as a dose of 0.5 mg/kg, or at a dose of about 0.6 mg/kg, such as at a dose of 0.6 mg/kg, or at a dose of about 0.7 mg/kg, such as at a dose of 0.7 mg/kg, or at a dose of about 0.8 mg/kg, such as at a dose of 0.8 mg/kg, or at a dose of about 0.9 mg/kg, such as at a dose of 0.9 mg/kg, or at a dose of about 1.0 mg/kg, such as at a dose of 1.0 mg/kg, or at a dose of about L1 mg/kg, such as at a dose of 1.1 mg/kg, or at a dose of about 1.2 mg/kg, such as at a dose of 1.2 mg/kg, or at a dose of about 1.3 mg/kg, such as at a dose of 1.3 mg/kg, or at a dose of about 1.4 mg/kg, such as at a dose of 1.4 mg/kg, or at a dose of about 1.5 mg/kg, such as or at a dose of 1.5 mg/kg, or at a dose of about 1.6 mg/kg, such as at a dose of 1.6 mg/kg, or at a dose of about 1.7 mg/kg, such as at a dose of 1.7 mg/kg, or at a dose of about 1.8 mg/kg, such as at a dose of 1.8 mg/kg, or at a dose of about 1.9 mg/kg, such as at a dose of 1.9 mg/kg, or at a dose of about 2.0 mg/kg, such as at a dose of 2.0 mg/kg.

In relation to the use of the antibody or ADC provided herein, wherein the number of cycles of 21 days or the number of cycles of 28 days is preferably between 2 and 48, such as between 2 and 36, such as between 2 and 24, such as between 2 and 15, such as between 2 and 12, such as 2 cycles, 3 cycles, 4 cycles, 5 cycles, 6 cycles, 7 cycles, 8 cycles, 9 cycles, 10 cycles, 11 cycles or 12 cycles.

The antibody or ADC for use as set forth above may administered for at least four treatment cycles of 28 days, wherein the antibody or ADC in each treatment cycle is administered once a week at a dose of about 0.45 mg/kg body weight, such as a dose of 0.45 mg/kg body weight, a dose of about 0.6 mg/kg body weight, a dose of 0.6 mg/kg body weight, a dose of about 0.8 mg/kg body weight, such as a dose of 0.8 mg/kg body weight, a dose of about 1.0 mg/kg body weight, such as dose of 1.0 mg/kg body weight, a dose of about 1.2 mg/kg body weight, such as a dose of 1.2 mg/kg body weight, a dose of about 1.4 mg/kg body weight, such as a dose of 1.4 mg/kg body weight, a dose of about 1.6 mg/kg body weight, such as a dose of 1.6 mg/kg body weight, a dose of about 1.8 mg/kg body weight, such as a dose of 1.8 mg/kg body weight, or a dose of about 2.0 mg/kg body weight, such as 2.0 mg/kg body weight for three consecutive weeks followed by a resting week without any administration of the antibody or ADC.

The conjugate may be administered to the subject at a dose of about 2.0-about 2.4 mg/kg body weight, such as 2.0-2.4 mg/kg body weight, once every three weeks or by weekly dosing of about 0.6-about 1.4 mg/kg body weight, such as 0.6-1.4 mg/kg body weight for three weeks, optionally followed by one treatment-free week.

The conjugate may administered to the subject at a dose of about 2.2 mg/kg body weight, such as 2.2 mg/kg body weight, once every three weeks or by weekly dosing of about 1.0 mg/kg body weight, such as 1.0 mg/kg body weight, for three weeks, optionally followed by one treatment-free week.

The conjugate may be administered to the subject by weekly dosing of about 0.4-about 1.0 mg/kg body weight, such as by weekly dosing of 0.4-1.0 mg/kg body weight.

The conjugate may be administered to the subject by weekly dosing of about 0.6-about 1.0 mg/kg body weight, such as by weekly dosing of 0.6-1.0 mg/kg body weight.

The conjugate may be administered to the subject by weekly dosing of about 0.4-about 0.8 mg/kg body weight, such as by weekly dosing of 0.4-0.8 mg/kg body weight.

The conjugate may be administered to the subject by weekly dosing of about 0.5-about 0.7 mg/kg body weight, such as by weekly dosing of 0.5-0.7 mg/kg body weight

The conjugate may be administered to the subject by weekly dosing of about 0.6 mg/kg body weight, such as by weekly dosing of 0.6 mg/kg body weight.

The route of administration may in particular be intravenous.

The treatment may be continued at least until said subject has experienced progression-free survival of at least about 1 month, such as at least 1 month; at least about 2 months, such as at least 2 months; at least about 3 months, such as at least 3 months; at least about 4 months, such as at least 4 months; at least about 5 months, such as at least 5 months; at least about 6 months, such as at least 6 months; at least about 7 months, such as at least 7 months; at least about 8 months, such as at least 8 months; at least about 9 months, such as at least 9 months; at least about 10 months, such as at least 10 months; at least about 11 months, such as at least 11 months; at least about 12 months, such as at least 12 months; at least about eighteen months, such as at least eighteen months; at least about two years, such as at least 2 years; at least about three years, such as at least three years; at least about four years, such as at least four years; or at least about five years, such as at least 5 years, after administration of the first dose of the conjugate.

The treatment may be continued until disease progression or unacceptable toxicity.

In a second aspect, the invention provides an antibody binding to human AXL or an antibody-drug conjugate (ADC) comprising an antibody binding to human AXL, for use in the manufacture of a medicament for treating cancer in a subject, wherein
said cancer is resistant to or is predicted to be or become resistant to;
said cancer has failed to respond to, or is predicted to fail to respond to; and/or
said subject has relapsed after or is predicted to relapse after
   treatment with an inhibitor of the interaction between a programmed cell death-1 (PD-1) receptor and its ligand.

It is to be understood that the above disclosure of features relating to the first aspect of the invention also applies to the second aspect of the invention

In particular, the antibody or ADC, for use in the manufacture of a medicament, wherein
the ligand is as defined above;
the inhibitor of the interaction between a programmed cell death-1 (PD-1) receptor and its ligand is as defined above;
the cancer is as defined above;
the subject is as defined above;
antibody or ADC is as defined above;
the formulation is as defined above; and/or
the amounts and frequencies in which the antibody or ADC is administered to said subject is as defined above.

A third aspect of the invention provides a method of treating cancer in a subject, wherein said cancer
is resistant to or is predicted to be or become resistant to;
has failed to respond to, or is predicted to fail to respond to; and/or
has relapsed after or is predicted to relapse after
   treatment with an inhibitor of the interaction between a programmed cell death-1 (PD-1) receptor and its ligand. The method comprises administering to said subject a therapeutically effective amount of an antibody binding to human AXL or an antibody-drug conjugate (ADC) comprising an antibody binding to human AXL.

In particular, the method of treating cancer according to the third aspect of the invention is a method, wherein
the ligand is as defined above;
the inhibitor of the interaction between a programmed cell death-1 (PD-1) receptor and its ligand is as defined above;
the cancer is as defined above;
the subject is as defined above;
antibody or ADC is as defined above;
the formulation is as defined above; and/or
the amounts and frequencies in which the antibody or ADC is administered to said subject is as defined above.

### Sequences

TABLE 2

| SEQ ID NO: | Name | Amino acid sequence | Comments |
|---|---|---|---|
| SEQ ID NO: 1 | 107 VH | EVQLLESGGGLVQPGGSLRLSCAASGFTFSSYAMNWVR | HCo12-BalbC |
| | | QAPGKGLEWVSTTSGSGASTYYADSVKGRFTISRDNSK | Ig1 domain |
| | | NTLYLQMNSLRAEDTAVYYCAKIWIAFDIWGQGTMVT | binding Ab |
| | | VSS | |
| SEQ ID NO: 2 | 107 VL | EIVLTQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGSSPYTFGQGTKLEIK | |
| SEQ ID NO: 3 | 140 VH | EVQLLESGGGLVQPGGSLRLSCAASGFTFSSYAMTWVR | |
| | | QAPGKGLEWVSAISISGASTFYADSVKGRFTISRDNSKN | |
| | | TLSLQMNSLRAEDTAVYFCRGYSGYVYDAFDIWGQGT | |
| | | MVTVSS | |
| SEQ ID NO: 4 | 140 VL | DIQMTQSPSSLSASVGDRVTITCRASQGISNWLAWYQ | |
| | | QKPEKAPKSLIYAASSLQSGVPSRFSGSGSGTDFTLTISSL | |
| | | QPEDFATYYCQQYNSYPLTFGGGTKVEIK | |
| SEQ ID NO: 5 | 148 VH | EVQLLESGGGLVQPGGSLRLSCAASGFTFSSYAMTWVR | HCo12-BalbC |
| | | QAPGKGLEWVSAISISGGSTFYADSVKGRFTISRDNSKN | Ig2 domain |
| | | TLYLQMNSLRAEDTAVYYCRGYSGYVYDAFDFWGQGT | binding Ab |
| | | MVTVSS | |
| SEQ ID NO: 6 | 148 VL | DIQMTQSPSSLSASVGDRVTITCRASQGISNWLAWYQ | |
| | | QKPEKAPKSLIYAASSLQSGVPSRFSGSGSGTDFTLTISSL | |
| | | QPEDFATYYCQQYNSYPLTFGGGTKVEIK | |
| SEQ ID NO: 7 | 154 VH | EVQLLDSGGGLVQPGGSLRLSCAASGFTFSSYAMSWVR | HCo12-BalbC |
| | | QAPGKGLEWVSAISIGGGNAYYADSVKGRFTISRDNSK | FN1 domain |
| | | NTLYLQMNSLRAADTAVYYCAKPGFIMVRGPLDYWGQ | binding Ab |
| | | GALVTVSS | |
| SEQ ID NO: 8 | 154-M103L VH | EVQLLDSGGGLVQPGGSLRLSCAASGFTFSSYAMSWVR | |
| | | QAPGKGLEWVSAISIGGGNAYYADSVKGRFTISRDNSK | |
| | | NTLYLQMNSLRAADTAVYYCAKPGFILVRGPLDYWGQ | |
| | | GALVTVSS | |
| SEQ ID NO: 9 | 154 VL | EIVLTQSPGTLSLSPGERATLSCRASQSVSNSYLAWYQQ | |
| | | KPGQAPRLLIYGASSRATGIPDRFSGSGSGTDFTLTISRLE | |
| | | PEDFAVYYCQQYGSSPYTFGQGTKLEIK | |
| SEQ ID NO: 10 | 171 VH | EVQLLESGGGLVQPGGSLRLSCAASGFTFSSYAMSWVR | HCo17-BalbC |
| | | QAPGKGLEWVSDISVSGGSTYYADSVKGRFTISRDNSK | Ig2 domain |
| | | NTLYLQMNSLRAEDTAVYYCAKEGYIWFGESLSYAFDI | binding Ab |
| | | WGQGTMVTVSS | |
| SEQ ID NO: 11 | 171 VL | EIVLTQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGRSFTFGPGTKVDIK | |
| SEQ ID NO: 12 | 172 VH | EVQLLESGGGLVQPGGSLRLSCAASGFTFSNYAMSWVR | |
| | | QAPGKGLEWVSDISVSGGSTYYADSVKGRFTISRDNSK | |
| | | NTLYLQMNSLRAEDTAVYYCAKEGYIWFGESLSYAFDI | |
| | | WGQGTMVTVSS | |
| SEQ ID NO: 13 | 172 VL | EIVLTQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGRSFTFGPGTKVDIK | |
| SEQ ID NO: 14 | 181 VH | EVQLLESGGGLVQPGGSLRLSCAASGFTFSSYAMSWVR | |
| | | QAPGKGLEWVSDISVSGGSTYYADSVKGRFTISRDNSK | |
| | | NTLYLHMNSLRAEDTAVYYCAKEGYIWFGESLSYAFDIW | |
| | | GQGTMVTVSS | |
| SEQ ID NO: 15 | 181 VH | EIVLTQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGRSFTFGPGTKVDIK | |
| SEQ ID NO: 16 | 183 VH | QVQLQQWGAGLLKPSETLSLTCAVYGGSFSGYYWSWI | HCo17-BalbC |
| | | RQPPGKGLEWIGEINQSGSTNYNPSLKSRVTISVDTSKN | FN1 domain |
| | | QFSLKLSSVTAADTSVYYCASGNWDHFFDYWGQGTLV | binding Ab |
| | | TVSS | |
| SEQ ID NO: 17 | 183-N52Q VH | QVQLQQWGAGLLKPSETLSLTCAVYGGSFSGYYWSWI | |
| | | RQPPGKGLEWIGEIQQSGSTNYNPSLKSRVTISVDTSKN | |
| | | QFSLKLSSVTAADTSVYYCASGNWDHFFDYWGQGTLV | |
| | | TVSS | |
| SEQ ID NO: 18 | 183 VL | DIQMTQSPSSVSASVGDRVTITCRASQGISSWLAWYQH | |
| | | KPGKAPKLLIYATSSLQSGVTSRFSGSGSGTDFTLTISSLQ | |
| | | PEDFATYYCQQAKSFPWTFGQGTKVEIK | |
| SEQ ID NO: 19 | 187 VH | QVPLQQWGAGLLKPSETLSLTCAVYGGSFSGYHWSWI | |
| | | RQPPGKGLEWIGEISHSGRTNYNPSLKSRVTISIDTSKNQ | |
| | | FSLKLSSVTAADTAVYYCASFITMIRGTIITHFDYWGQGT | |
| | | LVTVSS | |
| SEQ ID NO: 20 | 187 VL | DIQMTQSPSSLSASVGDRVTITCRASQGISSWLAWYQQ | |
| | | KPEKAPKSLIYAASSLQSGVPSRFSGSGSGTDFTLTISSLQ | |
| | | PEDFATYYCQQYHSYPYTFGQGTKLEIK | |
| SEQ ID NO: 21 | 608-01 VH | QVQLVQSGAEVKKPGSSVKVSCKASGGTFSSYAISWVR | |
| | | QAPGQGLEWMGRIIPIFGIANYVQKFQGRVTITADKSTS | |
| | | TAYMELSSLRAEDTAVYYCARRGDYYGSGSPDVFDIWG | |
| | | QGTMVTVSS | |
| SEQ ID NO: 22 | 608-01 VL | EIVLTQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGSSYTFGQGTKLEIK | |
| SEQ ID NO: 23 | 610-01 VH | QVQLVQSGAEVKKPGSSVKVSCKASGGTFSSYAISWVR | |
| | | QAPGQGLEWMGRIIPIFGIANYVQKFQGRVTITADKSTS | |
| | | TAYMELSSLRAEDTAVYYCARRGNYYGSGSPDVFDIWG | |
| | | QGTMVTVSS | |
| SEQ ID NO: 24 | 610-01 VL | EIVLTQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGSSYTFGQGTKLEIK | |
| SEQ ID NO: 25 | 613 VH | QVQLVQSGAEVKKPGSSVKVSCKASGGTFSSYAINWM | HCo20 |
| | | RQAPGQGLEWMGRIIPIFGIVNYAQKFQGRVTLTADKS | Ig1 domain |
| | | TSTAYMELSSLRSEDTAVYYCARRGNYYGSGSPDVFDIW | binding Ab |
| | | GQGTMVTVSS | |
| SEQ ID NO: 26 | 613 VL | EIVLTQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGSSYTFGQGTKLEIK | |
| SEQ ID NO: 27 | 613-08 VH | QVQLVQSGAEVKKPGSSVKVSCKASGGTFSSYAINWM | |
| | | RQAPGQGLEWMGRIIPIFGIVNYAQKFQGRVTLTADKS | |
| | | TSTAYMELSSLRSEDTAVYYCARRGNYYGSGSPDVFDIW | |
| | | GQGTMVTVSS | |
| SEQ ID NO: 28 | 613-08 VL | EIVLTQSPATLSLSPGERATLSCRASQSVSSYLAWYQQKP | |
| | | GQAPRLLIYDASNRATGIPARFSGSGSGTDFTLTISSLEPE | |
| | | DFAVYYCQQRSNWLTFGGGTKVEIK | |
| SEQ ID NO: 29 | 620-06 VH | QVQLVQSGAEVKKPGSSVKVSCKASGGTFSSYAISWVR | |
| | | QAPGQGLEWMGRIIPIFGIANYAQKFQGRVTITADKSTS | |
| | | TAYMELSSLRSEDTAVYYCARRGNYYGSGSPDVFDIWG | |
| | | QGTMVTVSS | |
| SEQ ID NO: 30 | 620-06 VL | EIVLTQSPGTLSLSPGERATLSCRASQSVSSSYLAWYQQK | |
| | | PGQAPRLLIYGASSRATGIPDRFSGSGSGTDFTLTISRLEP | |
| | | EDFAVYYCQQYGSSYTFGQGTKLEIK | |
| SEQ ID NO: 31 | 726 VH | QVQLQQWGAGLLKPSETLSLTCAIDGGSFSGYYWSWIR | HCo17-BalbC |
| | | QPPGKGLEWIGEISHSGRTNYNPSLKSRVTISIDTSKNQF | FN2 domain |
| | | SLKLSSVAAADTAVYYCARFITMIRGAIITHFDYWGQGA | binding Ab |
| | | LVTVSS | |
| SEQ ID NO: 32 | 726-M101L VH | QVQLQQWGAGLLKPSETLSLTCAIDGGSFSGYYWSWIR | |
| | | QPPGKGLEWIGEISHSGRTNYNPSLKSRVTISIDTSKNQF | |
| | | SLKLSSVAAADTAVYYCARFITLIRGAIITHFDYWGQGAL | |
| | | VTVSS | |
| SEQ ID NO: 33 | 726 VL | DIQMTQSPSSLSASVGDRVTITCRASQGISSWLAWYQQ | |
| | | KPEKAPKSLIYAASSLQSGVPSRFSGSGSGTDFTLTISSLQ | |
| | | PEDFATYYCQQYHSYPYTFGQGTKLEIK | |
| SEQ ID NO: 34 | 733 VH | QVQLVESGGGVVQPGRSLRLSCAASGFSFSTYAMHWV | HCo17-BalbC |
| | | RQAPGKGLEWVAVISYDGDNKYSADSVKGRFTISRDNS | FN1 domain |
| | | KNTLYLQMNSLRAEDTAVYYCARGRKLGIDAFDIWGQG | binding Ab |
| | | TMVTVSS | |
| SEQ ID NO: 35 | 733 VL | AIQLTQSPSSLSASVGDRVTITCRASQGISSALAWYQQK | |
| | | PGKAPKLLIYDASSLESGVPSRFSGSGSGTDFTLTISGLQP | |
| | | EDFATYYCQQFNSYPFTFGPGTKVDIK | |
| SEQ ID NO: 36 | 107 VH CDR1 | GFTFSSYA | |
| SEQ ID NO: 37 | 107 VH CDR2 | TSGSGAST | |
| SEQ ID NO: 38 | 107 VH CDR3 | AKIWIAFDI | |
| SEQ ID NO: 39 | 107 VL CDR1 | QSVSSSY | |
| | 107 VL CDR2 | GAS | |
| SEQ ID NO: 40 | 107 VL CDR3 | QQYGSSPYT | |
| SEQ ID NO: 41 | 140 VH CDR1 | GFTFSSYA | |
| SEQ ID NO: 42 | 140 VH CDR2 | ISISGAST | |
| SEQ ID NO: 43 | 140 VH CDR3 | RGYSGYVYDAFDI | |
| SEQ ID NO: 44 | 140 VL CDR1 | QGISNW | |
| | 140 VL CDR2 | AAS | |
| SEQ ID NO: 45 | 140 VL CDR3 | QQYNSYPLT | |
| SEQ ID NO: 46 | 148 VH CDR1 | GFTFSSYA | |
| SEQ ID NO: 47 | 148 VH CDR2 | ISISGGST | |
| SEQ ID NO: 48 | 148 VH CDR3 | RGYSGYVYDAFDF | |
| SEQ ID NO: 49 | 148 VL CDR1 | QGISNW | |
| | 148 VL CDR2 | AAS | |
| SEQ ID NO: 50 | 148 VL CDR3 | QQYNSYPLT | |
| SEQ ID NO: 51 | 154 VH CDR1 | GFTFSSYA | |
| SEQ ID NO: 52 | 154 VH CDR2 | ISIGGGNA | |
| SEQ ID NO: 53 | 154 VH CDR3 | AKPGFIMVRGPLDY | |
| SEQ ID NO: 54 | 154-M103L VH | AKPGFILVRGPLDY | |
| | CDR3 | | |
| SEQ ID NO: 55 | 154 VL CDR1 | QSVSNSY | |
| | 154 VL CDR2 | GAS | |
| SEQ ID NO: 56 | 154 VL CDR3 | QQYGSSPYT | |
| SEQ ID NO: 57 | 171 VH CDR1 | GFTFSSYA | |
| SEQ ID NO: 58 | 171 VH CDR2 | ISVSGGST | |
| SEQ ID NO: 59 | 171 VH CDR3 | AKEGYIWFGESLSYAFDI | |
| SEQ ID NO: 60 | 171 VL CDR1 | QSVSSSY | |
| | 171 VL CDR2 | GAS | |
| SEQ ID NO: 61 | 171 VL CDR3 | QQYGRSFT | |
| SEQ ID NO: 62 | 172 VH CDR1 | GFTFSNYA | |
| SEQ ID NO: 63 | 172 VH CDR2 | ISVSGGST | |
| SEQ ID NO: 64 | 172 VH CDR3 | AKEGYIWFGESLSYAFDI | |
| SEQ ID NO: 65 | 172 VL CDR1 | QSVSSSY | |
| | 172 VL CDR2 | GAS | |
| SEQ ID NO: 66 | 172 VL CDR3 | QQYGRSFT | |
| SEQ ID NO: 67 | 181 VH CDR1 | GFTFSSYA | |
| SEQ ID NO: 68 | 181 VH CDR2 | ISVSGGST | |
| SEQ ID NO: 69 | 181 VH CDR3 | AKEGYIWFGESLSYAFDI | |
| SEQ ID NO: 70 | 181 VL CDR1 | QSVSSSY | |
| | 181 VL CDR2 | GAS | |
| SEQ ID NO: 71 | 181 VL CDR3 | QQYGRSFT | |
| SEQ ID NO: 72 | 183 VH CDR1 | GGSFSGYY | |
| SEQ ID NO: 73 | 183 VH CDR2 | INQSGST | |
| SEQ ID NO: 74 | 183-N52Q VH | IQQSGST | |
| | CDR2 | | |
| SEQ ID NO: 75 | 183 VH CDR3 | ASGNWDHFFDY | |
| SEQ ID NO: 76 | 183 VL CDR1 | QGISSW | |
| | 183 VL CDR2 | ATS | |
| SEQ ID NO: 77 | 183 VL CDR3 | QQAKSFPWT | |
| SEQ ID NO: 78 | 187 VH CDR1 | GGSFSGYH | |
| SEQ ID NO: 79 | 187 VH CDR2 | ISHSGRT | |
| SEQ ID NO: 80 | 187 VH CDR3 | ASFITMIRGTIITHFDY | |
| SEQ ID NO: 81 | 187 VL CDR1 | QGISSW | |
| | 187 VL CDR2 | AAS | |
| SEQ ID NO: 82 | 187 VL CDR3 | QQYHSYPYT | |
| SEQ ID NO: 83 | 608-01 VH CDR1 | GGTFSSYA | |
| SEQ ID NO: 84 | 608-01 VH CDR2 | IIPIFGIA | |
| SEQ ID NO: 85 | 608-01 VH CDR3 | ARRGDYYGSGSPDVFDI | |
| SEQ ID NO: 86 | 608-01 VL CDR1 | QSVSSSY | |
| | 608-01 VL CDR2 | GAS | |
| SEQ ID NO: 87 | 608-01 VL CDR3 | QQYGSSYT | |
| SEQ ID NO: 88 | 610-01 VH CDR1 | GGTFSSYA | |
| SEQ ID NO: 89 | 610-01 VH CDR2 | IIPIFGIA | |
| SEQ ID NO: 90 | 610-01 VH CDR3 | ARRGNYYGSGSPDVFDI | |
| SEQ ID NO: 91 | 610-01 VL CDR1 | QSVSSSY | |
| | 610-01 VL CDR2 | GAS | |
| SEQ ID NO: 92 | 610-01 VL CDR3 | QQYGSSYT | |
| SEQ ID NO: 93 | 613 VH CDR1 | GGTFSSYA | |
| SEQ ID NO: 94 | 613 VH CDR2 | IIPIFGIV | |
| SEQ ID NO: 95 | 613 VH CDR3 | ARRGNYYGSGSPDVFDI | |
| SEQ ID NO: 96 | 613 VL CDR1 | QSVSSSY | |
| | 613 VL CDR2 | GAS | |
| SEQ ID NO: 97 | 613 VL CDR3 | QQYGSSYT | |
| SEQ ID NO: 98 | 613-08 VH CDR1 | GGTFSSYA | |
| SEQ ID NO: 99 | 613-08 VH CDR2 | IIPIFGIV | |
| SEQ ID NO: 100 | 613-08 VH CDR3 | ARRGNYYGSGSPDVFDI | |
| SEQ ID NO: 101 | 613-08 VL CDR1 | QSVSSY | |
| | 613-08 VL CDR2 | DAS | |
| SEQ ID NO: 102 | 613-08 VL CDR3 | QQRSNWLT | |
| SEQ ID NO: 103 | 620-06 VH CDR1 | GGTFSSYA | |
| SEQ ID NO: 104 | 620-06 VH CDR2 | IIPIFGIA | |
| SEQ ID NO: 105 | 620-06 VH CDR3 | ARRGNYYGSGSPDVFDI | |
| SEQ ID NO: 106 | 620-06 VL CDR1 | QSVSSSY | |
| | 620-06 VL CDR2 | GAS | |
| SEQ ID NO: 107 | 620-06 VL CDR3 | QQYGSSYT | |
| SEQ ID NO: 108 | 726 VH CDR1 | GGSFSGYY | |
| SEQ ID NO: 109 | 726 VH CDR2 | ISHSGRT | |
| SEQ ID NO: 110 | 726 VH CDR3 | ARFITMIRGAIITHFDY | |
| SEQ ID NO: 111 | 726-M 101L VH | ARFITLIRGAIITHFDY | |
| | CDR3 | | |
| SEQ ID NO: 112 | 726 VL CDR1 | QGISSW | |
| | 726 VL CDR2 | AAS | |
| SEQ ID NO: 113 | 726 VL CDR3 | QQYHSYPYT | |
| SEQ ID NO: 114 | 733 VH CDR1 | GFSFSTYA | |
| SEQ ID NO: 115 | 733 VH CDR2 | ISYDGDNK | |
| SEQ ID NO: 116 | 733 VH CDR3 | ARGRKLGIDAFDI | |
| SEQ ID NO: 117 | 733 VL CDR1 | QGISSA | |
| | 733 VL CDR2 | DAS | |
| SEQ ID NO: 118 | 733 VL CDR3 | QQFNSYPFT | |
| SEQ ID NO: 119 | Ig2 domain VH | ISISGXST-wherein X is A or G | |
| | CDR2 | | |
| SEQ ID NO: 120 | Ig2 domain VH | RGYSGYVYDAFDX-wherein Xis I or F | |
| | CDR3 | | |
| SEQ ID NO: 121 | FN2 domain VH | GGSFSGYX-wherein X is H or Y | |
| | CDR1 | | |
| SEQ ID NO: 122 | FN2 domain VH | AX1FITMIRGX2IITHFDY-wherein X1 is S or R; | |
| | CDR3 | and X2 is T or A | |
| SEQ ID NO: 123 | FN1 domain VH | GFTFSXYA-wherein X is S or N | |
| | CDR1 | | |
| SEQ ID NO: 124 | FN1 domain VH | ISVSGGST | |
| | CDR2 | | |
| SEQ ID NO: 125 | FN1 domain VH | AKEGYIWFGESLSYAFDI | |
| | CDR3 | | |
| SEQ ID NO: 126 | Ig1 domain VH | IIPIFGIX-wherein X is A or V | |
| | CDR2 | | |
| SEQ ID NO: 127 | Ig1 domain VH | ARRGXYYGSGSPDVFDI-wherein X is D or N | |
| | CDR3 | | |
| SEQ ID NO: 128 | Ig1 domain VL | QSVXSSY-wherein X is S or del | |
| | CDR1 | | |
| | Ig1 domain VL | XAS-wherein X is D or G | |
| | CDR2 | | |
| SEQ ID NO: 129 | Ig1 domain VL | QQX1X2X3X4X5T-wherein X1 is R or Y; X2 is | |
| | CDR3 | S or G; X3 is N or 5; X4 is W or 5; and | |
| | | X5 is L or Y | |
| SEQ ID NO: 130 | Human AXL | MAWRCPRMGRVPLAWCLALCGWACMAPRGTQAEES | |
| | protein | PFVGNPGNITGARGLTGTLRCQLQVQGEPPEVHWLRD | |
| | (Swissprot | GQILELADSTQTQVPLGEDEQDDWIVVSQLRITSLQLSD | |
| | P30530) | TGQYQCLVFLGHQTFVSQPGYVGLEGLPYFLEEPEDRTV | |
| | | AANTPFNLSCQAQGPPEPVDLLWLQDAVPLATAPGHG | |
| | | PQRSLHVPGLNKTSSFSCEAHNAKGVTTSRTATITVLPQ | |
| | | QPRNLHLVSRQPTELEVAWTPGLSGIYPLTHCTLQAVLS | |
| | | DDGMGIQAGEPDPPEEPLTSQASVPPHQLRLGSLHPHT | |
| | | PYHIRVACTSSQGPSSWTHWLPVETPEGVPLGPPENISA | |
| | | TRNGSQAFVHWQEPRAPLQGTLLGYRLAYQGQDTPEV | |
| | | LMDIGLRQEVTLELQGDGSVSNLTVCVAAYTAAGDGP | |
| | | WSLPVPLEAWRPGQAQPVHQLVKEPSTPAFSWPWWY | |
| | | VLLGAVVAAACVLILALFLVHRRKKETRYGEVFEPTVERG | |
| | | ELVVRYRVRKSYSRRTTEATLNSLGISEELKEKLRDVMVD | |
| | | RHKVALGKTLGEGEFGAVMEGQLNQDDSILKVAVKTM | |
| | | KIAICTRSELEDFLSEAVCMKEFDHPNVMRLIGVCFQGS | |
| | | ERESFPAPVVILPFMKHGDLHSFLLYSRLGDQPVYLPTQ | |
| | | MLVKFMADIASGMEYLSTKRFIHRDLAARNCMLNENM | |
| | | SVCVADFGLSKKIYNGDYYRQGRIAKMPVKWIAIESLAD | |
| | | RVYTSKSDVWSFGVTMWEIATRGQTPYPGVENSEIYDY | |
| | | LRQGNRLKQPADCLDGLYALMSRCWELNPQDRPSFTE | |
| | | LREDLENTLKALPPAQEPDEILYVNMDEGGGYPEPPGA | |
| | | AGGADPPTQPDPKDSCSCLTAAEVHPAGRYVLCPSTTP | |
| | | SPAQPADRGSPAAPGQEDGA | |
| SEQ ID NO: 131 | Mus musculus | MAWRCPRMGRVPLAWCLALCGWACMYPYDVPDYAA | |
| | AXL | HKDTQTEAGSPFVGNPGNITGARGLTGTLRCELQVQGE | |
| | | PPEVVWLRDGQILELADNTQTQVPLGEDWQDEWKVV | |
| | | SQLRISALQLSDAGEYQCMVHLEGRTFVSQPGFVGLEG | |
| | | LPYFLEEPEDKAVPANTPFNLSCQAQGPPEPVTLLWLQ | |
| | | DAVPLAPVTGHSSQHSLQTPGLNKTSSFSCEAHNAKGV | |
| | | TTSRTATITVLPQRPHHLHVVSRQPTELEVAWTPGLSGI | |
| | | YPLTHCNLQAVLSDDGVGIWLGKSDPPEDPLTLQVSVP | |
| | | PHQLRLEKLLPHTPYHIRISCSSSQGPSPWTHWLPVETTE | |
| | | GVPLGPPENVSAMRNGSQVLVRWQEPRVPLQGTLLGY | |
| | | RLAYRGQDTPEVLMDIGLTREVTLELRGDRPVANLTVSV | |
| | | TAYTSAGDGPWSLPVPLEPWRPGQGQPLHHLVSEPPP | |
| | | RAFSWPWWYVLLGAVVAAACVLILALFLVHRRKKETRY | |
| | | GEVFEPTVERGELVVRYRVRKSYSRRTTEATLNSLGISEEL | |
| | | KEKLRDVMVDRHKVALGKTLGEGEFGAVMEGQLNQD | |
| | | DSILKVAVKTMKIAICTRSELEDFLSEAVCMKEFDHPNV | |
| | | MRLIGVCFQGSERESFPAPVVILPFMKHGDLHSFLLYSRL | |
| | | GDQPVYLPTQMLVKFMADIASGMEYLSTKRFIHRDLAA | |
| | | RNCMLNENMSVCVADFGLSKKIYNGDYYRQGRIAKMP | |
| | | VKWIAIESLADRVYTSKSDVWSFGVTMWEIATRGQTPY | |
| | | PGVENSEIYDYLRQGNRLKQPADCLDGLYALMSRCWEL | |
| | | NPQDRPSFTELREDLENTLKALPPAQEPDEILYVNMDEG | |
| | | GGYPEPPGAAGGADPPTQPDPKDSCSCLTAAEVHPAG | |
| | | RYVLCPSTTPSPAQPADRGSPAAPGQEDGA | |
| SEQ ID NO: 132 | Homo sapiens | MAWRCPRMGRVPLAWCLALCGWACMAPRGTQAEES | |
| | AXL-Mus | PFVGNPGNITGARGLTGTLRCQLQVQGEPPEVHWLRD | |
| | musculus Ig1 | GQILELADSTQTQVPLGEDEQDDWIVVSQLRITSLQLSD | |
| | domain | TGQYQCLVFLGHQTFVSQPGYVGLEGLPYFLEEPEDKAV | |
| | | PANTPFNLSCQAQGPPEPVTLLWLQDAVPLAPVTGHSS | |
| | | QHSLQTPGLNKTSSFSCEAHNAKGVTTSRTATITVLPQQ | |
| | | PRNLHLVSRQPTELEVAWTPGLSGIYPLTHCTLQAVLSD | |
| | | DGMGIQAGEPDPPEEPLTSQASVPPHQLRLGSLHPHTP | |
| | | YHIRVACTSSQGPSSWTHWLPVETPEGVPLGPPENISAT | |
| | | RNGSQAFVHWQEPRAPLQGTLLGYRLAYQGQDTPEVL | |
| | | MDIGLRQEVTLELQGDGSVSNLTVCVAAYTAAGDGPW | |
| | | SLPVPLEAWRPGQAQPVHQLVKEPSTPAFSWPWWYV | |
| | | LLGAVVAAACVLILALFLVHRRKKETRYGEVFEPTVERGE | |
| | | LVVRYRVRKSYSRRTTEATLNSLGISEELKEKLRDVMVDR | |
| | | HKVALGKTLGEGEFGAVMEGQLNQDDS | |
| | | ILKVAVKTMKIAICTRSELEDFLSEAVCMKEFDHPNVMR | |
| | | LIGVCFQGSERESFPAPVVILPFMKHGDLHSFLLYSRLGD | |
| | | QPVYLPTQMLVKFMADIASGMEYLSTKRFIHRDLAARN | |
| | | CMLNENMSVCVADFGLSKKIYNGDYYRQGRIAKMPVK | |
| | | WIAIESLADRVYTSKSDVWSFGVTMWEIATRGQTPYPG | |
| | | VENSEIYDYLRQGNRLKQPADCLDGLYALMSRCWELNP | |
| | | QDRPSFTELREDLENTLKALPPAQEPDEILYVNMDEGG | |
| | | GYPEPPGAAGGADPPTQPDPKDSCSCLTAAEVHPAGRY | |
| | | VLCPSTTPSPAQPADRGSPAAPGQEDGA | |
| SEQ ID NO: 133 | Homo sapiens | MAWRCPRMGRVPLAWCLALCGWACMAPRGTQAEES | |
| | AXL-Mus | PFVGNPGNITGARGLTGTLRCQLQVQGEPPEVHWLRD | |
| | musculus Ig2 | GQILELADSTQTQVPLGEDEQDDWIVVSQLRITSLQLSD | |
| | domain | TGQYQCLVFLGHQTFVSQPGYVGLEGLPYFLEEPEDKAV | |
| | | PANTPFNLSCQAQGPPEPVTLLWLQDAVPLAPVTGHSS | |
| | | QHSLQTPGLNKTSSFSCEAHNAKGVTTSRTATITVLPQQ | |
| | | PRNLHLVSRQPTELEVAWTPGLSGIYPLTHCTLQAVLSD | |
| | | DGMGIQAGEPDPPEEPLTSQASVPPHQLRLGSLHPHTP | |
| | | YHIRVACTSSQGPSSWTHWLPVETPEGVPLGPPENISAT | |
| | | RNGSQAFVHWQEPRAPLQGTLLGYRLAYQGQDTPEVL | |
| | | MDIGLRQEVTLELQGDGSVSNLTVCVAAYTAAGDGPW | |
| | | SLPVPLEAWRPGQAQPVHQLVKEPSTPAFSWPWWYV | |
| | | LLGAVVAAACVLILALFLVHRRKKETRYGEVFEPTVERGE | |
| | | LVVRYRVRKSYSRRTTEATLNSLGISEELKEKLRDVMVDR | |
| | | HKVALGKTLGEGEFGAVMEGQLNQDDS | |
| | | ILKVAVKTMKIAICTRSELEDFLSEAVCMKEFDHPNVMR | |
| | | LIGVCFQGSERESFPAPVVILPFMKHGDLHSFLLYSRLGD | |
| | | QPVYLPTQMLVKFMADIASGMEYLSTKRFIHRDLAARN | |
| | | CMLNENMSVCVADFGLSKKIYNGDYYRQGRIAKMPVK | |
| | | WIAIESLADRVYTSKSDVWSFGVTMWEIATRGQTPYPG | |
| | | VENSEIYDYLRQGNRLKQPADCLDGLYALMSRCWELNP | |
| | | QDRPSFTELREDLENTLKALPPAQEPDEILYVNMDEGG | |
| | | GYPEPPGAAGGADPPTQPDPKDSCSCLTAAEVHPAGRY | |
| | | VLCPSTTPSPAQPADRGSPAAPGQEDGA | |
| SEQ ID NO: 134 | Homo sapiens | MAWRCPRMGRVPLAWCLALCGWACMAPRGTQAEES | |
| | AXL- | PFVGNPGNITGARGLTGTLRCQLQVQGEPPEVHWLRD | |
| | Mus musculus | GQILELADSTQTQVPLGEDEQDDWIVVSQLRITSLQLSD | |
| | FN1 domain | TGQYQCLVFLGHQTFVSQPGYVGLEGLPYFLEEPEDRTV | |
| | | AANTPFNLSCQAQGPPEPVDLLWLQDAVPLATAPGHG | |
| | | PQRSLHVPGLNKTSSFSCEAHNAKGVTTSRTATITVLPQ | |
| | | RPHHLHVVSRQPTELEVAWTPGLSGIYPLTHCNLQAVLS | |
| | | DDGVGIWLGKSDPPEDPLTLQVSVPPHQLRLEKLLPHTP | |
| | | YHIRISCSSSQGPSPWTHWLPVETTEGVPLGPPENISAT | |
| | | RNGSQAFVHWQEPRAPLQGTLLGYRLAYQGQDTPEVL | |
| | | MDIGLRQEVTLELQGDGSVSNLTVCVAAYTAAGDGPW | |
| | | SLPVPLEAWRPGQAQPVHQLVKEPSTPAFSWPWWYV | |
| | | LLGAVVAAACVLILALFLVHRRKKETRYGEVFEPTVERGE | |
| | | LVVRYRVRKSYSRRTTEATLNSLGISEELKEKLRDVMVDR | |
| | | HKVALGKTLGEGEFGAVMEGQLNQDDS | |
| | | ILKVAVKTMKIAICTRSELEDFLSEAVCMKEFDHPNVMR | |
| | | LIGVCFQGSERESFPAPVVILPFMKHGDLHSFLLYSRLGD | |
| | | QPVYLPTQMLVKFMADIASGMEYLSTKRFIHRDLAARN | |
| | | CMLNENMSVCVADFGLSKKIYNGDYYRQGRIAKMPVK | |
| | | WIAIESLADRVYTSKSDVWSFGVTMWEIATRGQTPYPG | |
| | | VENSEIYDYLRQGNRLKQPADCLDGLYALMSRCWELNP | |
| | | QDRPSFTELREDLENTLKALPPAQEPDEILYVNMDEGG | |
| | | GYPEPPGAAGGADPPTQPDPKDSCSCLTAAEVHPAGRY | |
| | | VLCPSTTPSPAQPADRGSPAAPGQEDGA | |
| SEQ ID NO: 135 | Homo sapiens | MAWRCPRMGRVPLAWCLALCGWACMAPRGTQAEES | |
| | AXL- | PFVGNPGNITGARGLTGTLRCQLQVQGEPPEVHWLRD | |
| | Mus musculus | GQILELADSTQTQVPLGEDEQDDWIVVSQLRITSLQLSD | |
| | FN2 domain | TGQYQCLVFLGHQTFVSQPGYVGLEGLPYFLEEPEDRTV | |
| | | AANTPFNLSCQAQGPPEPVDLLWLQDAVPLATAPGHG | |
| | | PQRSLHVPGLNKTSSFSCEAHNAKGVTTSRTATITVLPQ | |
| | | QPRNLHLVSRQPTELEVAWTPGLSGIYPLTHCTLQAVLS | |
| | | DDGMGIQAGEPDPPEEPLTSQASVPPHQLRLGSLHPHT | |
| | | PYHIRVACTSSQGPSSWTHWLPVETPEGVPLGPPENVS | |
| | | AMRNGSQVLVRWQEPRVPLQGTLLGYRLAYRGQDTPE | |
| | | VLMDIGLIREVILELRGDRPVANLIVSVTAYTSAGDGP | |
| | | WSLPVPLEPWRPGQGQPLHHLVSEPPPRAFSWPWWY | |
| | | VLLGAVVAAACVLILALFLVHRRKKETRYGEVFEPTVERG | |
| | | ELVVRYRVRKSYSRRTTEATLNSLGISEELKEKLRDVMVD | |
| | | RHKVALGKTLGEGEFGAVMEGQLNQDDSILKVAVKTM | |
| | | KIAICTRSELEDFLSEAVCMKEFDHPNVMRLIGVCFQGS | |
| | | ERESFPAPVVILPFMKHGDLHSFLLYSRLGDQPVYLPTQ | |
| | | MLVKFMADIASGMEYLSTKRFIHRDLAARNCMLNENM | |
| | | SVCVADFGLSKKIYNGDYYRQGRIAKMPVKWIAIESLAD | |
| | | RVYTSKSDVWSFGVTMWEIATRGQTPYPGVENSEIYDY | |
| | | LRQGNRLKQPADCLDGLYALMSRCWELNPQDRPSFTE | |
| | | LREDLENTLKALPPAQEPDEILYVNMDEGGGYPEPPGA | |
| | | AGGADPPTQPDPKDSCSCLTAAEVHPAGRYVLCPSTTP | |
| | | SPAQPADRGSPAAPGQEDGA | |
| SEQ ID NO: 136 | 511 VH | EVQLLESGGGLVQPGGSLRLSCAASGFTFSSYAMNWVR | Ig2 domain |
| | | QAPGKGLEWVSGISGSGGHTYHADSVKGRFTISRDNSK | binding Ab |
| | | NTLYLQMNSLRAEDTAVYYCAKDRYDILTGYYNLLDYW | |
| | | GQGTLVTVSS | |
| SEQ ID NO: 137 | 511 VH CDR1 | GFTFSSYA | |
| SEQ ID NO: 138 | 511 VH CDR2 | ISGSGGHT | |
| SEQ ID NO: 139 | 511 VH CDR3 | AKDRYDILTGYYNLLDY | |
| SEQ ID NO: 140 | 511 VL | DIQMTQSPSSLSASVGDRVTITCRASQGISSWLAWYQQ | |
| | | KPEEAPKSLIYAASSLQSGVPSRFSGSGSGTDFTLTISSLQ | |
| | | PEDFATYYCQQYNSYPLTFGGGAKVEIK | |
| SEQ ID NO: 141 | 511 VL CDR1 | QGISSW | |
| | 511 VL CDR2 | AAS | |
| SEQ ID NO: 142 | 511 VL CDR3 | QQYNSYPLT | |
| SEQ ID NO: 143 | 061 VH | QVQLVQSGAEVKKPGASVKVSCKASGYAFTGYGISWVR | Ig1 domain |
| | | QAPGQGLEWIGWISAYNGNTNYVQNLQDRVTMTTDT | binding Ab |
| | | STSTAYMELRSLRSDDTAVYYCARDHISMLRGIIIRNYW | |
| | | GQGTLVTVSS | |
| SEQ ID NO: 144 | 061 VL | EIVLTQSPATLSLSPGERATLSCRASQSVSSYLAWYQQKP | |
| | | GQAPRLLIYDASNRATGIPARFSGSGSGTDFTLTISSLEPE | |
| | | DFAVYYCQQRSSWPRLTFGGGTKVEIK | |
| SEQ ID NO: 145 | 137 VH | QVQLVQSGAEVKKPGSSVKVSCKASGGTFSRYAISWVR | |
| | | QAPGQGLEWMGRIIPIVGIANYAQKFQGRVTLTADKST | |
| | | STAYMELSSLRSEDTAVYYCAREAGYSSSWYAEYFQHW | |
| | | GQGTLVTVSS | |
| SEQ ID NO: 146 | 137 VL | EIVLTQSPGTLSLSPGERATLSCRASQSVSSNYLAWYQQ | |
| | | KPGQAPRLLIYGASSRATGFPDRFSGSGSGTDFTLTISRL | |
| | | EPEDFAVYYCQQYGSSPYTFGQGTKLEIK | |
| SEQ ID NO: 147 | Cynomolgus | AWRCPRMGRVPLAWCLALCGWVCMAPRGTQAEESP | |
| | monkey AXL | FVGNPGNITGARGLTGTLRCQLQVQGEPPEVHWLRDG | |
| | (GenBank number | QILELADSTQTQVPLGEDEQDDWIVVSQLRIASLQLSDA | |
| | HB387229.1) | GQYQCLVFLGHQNFVSQPGYVGLEGLPYFLEEPEDRTV | |
| | | AANTPFNLSCQAQGPPEPVDLLWLQDAVPLATAPGHG | |
| | | PQRNLHVPGLNKTSSFSCEAHNAKGVTTSRTATITVLPQ | |
| | | QPRNLHLVSRQPTELEVAWTPGLSGIYPLTHCTLQAVLS | |
| | | DDGMGIQAGEPDPPEEPLTLQASVPPHQLRLGSLHPHT | |
| | | PYHIRVACTSSQGPSSWTHWLPVETPEGVPLGPPENISA | |
| | | TRNGSQAFVHWQEPRAPLQGTLLGYRLAYQGQDTPEV | |
| | | LMDIGLRQEVTLELQGDGSVSNLTVCVAAYTAAGDGP | |
| | | WSLPVPLEAWRPGQAQPVHQLVKETSAPAFSWPWW | |
| | | VILLGAVVAAACVLILALFLVHRRKKETRYGEVFEPTVER | |
| | | GELVVRYRVRKSYSRRTTEATLNSLGISEELKEKLRDVMV | |
| | | DRHKVALGKTLGEGEFGAVMEGQLNQDDSILKVAVKT | |
| | | MKIAICTRSELEDFLSEAVCMKEFDHPNVMRLIGVCFQG | |
| | | SERESFPAPVVILPFMKHGDLHSFLLYSRLGDQPVYLPTQ | |
| | | MLVKFMADIASGMEYLSTKRFIHRDLAARNCMLNENM | |
| | | SVCVADFGLSKKIYNGDYYRQGRIAKMPVKWIAIESLAD | |
| | | RVYTSKSDVWSFGVTMWEIATRGQTPYPGVENSEIYDY | |
| | | LRQGNRLKQPADCLDGLYALMSRCWELNPQDRPSFTE | |
| | | LREDLENTLKALPPAQEPDEILYVNMDEGGGYPEPPGA | |
| | | AGGADPPTQLDPKDSCSCLTSAEVHPAGRYVLCPSTAPS | |
| | | PAQPADRGSPAAPGQEDGA | |

The present invention is further illustrated by the following examples which should not be construed as further limiting the scope of the present disclosure.

### EXAMPLES

### Example 1—Axl Expression in Tumor Tissues Derived from Patients Who were Resistant to or Relapsed from PD-1 or PD-L1 Targeting Therapy

### Immunohistochemistry

Expression of AXL is evaluated in freshly cut paraffin embedded and formalin fixated (FFPE) tumor tissues obtained from patients with solid tumors, such as esophageal cancer, non-small cell lung cancer (NSCLC), squamous cell carcinoma of the head and neck (SCCHN), bladder cancer, prostate cancer, ovarian/fallopian tube cancer, cervical cancer, endometrial cancer, melanoma, colorectal cancer (CRC), pancreatic cancer, renal cell carcinoma (RCC), small-cell lung cancer (SCLC), liver cancer, gastro-intestinal cancer, breast cancer, glioblastoma, mesothelioma merkel cell carcinoma and sarcoma, who were resistant or refractory to or had relapsed from PD-1 or PD-L1 targeting therapy. Staining is performed either manually in Sequenza Slide Racks (Ted Pella Inc., Redding, Calif., USA; cat. no. 36105) or on a Ventana BenchMark Ultra (IHC Autostainer) with an anti-human Axl rabbit polyclonal antibody H-124 (Santa Cruz, Dallas, Tex., USA).

Prior to staining, FFPE tissue slides are deparaffinized in 100% xylene (Sigma-Aldrich, cat. no. 16446; three times, 5 min.) and dehydrated in 96% ethanol (Sigma Aldrich, cat. no. 32294; two times, 5 min.) at room temperature. Thereafter, antigen retrieval is performed. IHC slides are incubated in citrate buffer (pH6; DAKO; cat. no. 52369) for 5 min. and blocked for endogenous peroxidase in citrate/phosphate buffer (0.43 M citric acid, 0.35 M Na₂HPO₄.2H₂O; pH5.8) at RT for 15 min. Slides are incubated in 10% normal human serum (CLB/Sanquin, cat. no. K1146) in PBS prior to incubation with primary antibodies. Axl expression is determined by incubation with rabbit polyclonal anti-human Axl antibody H-124 in PBS supplemented with 2% normal human serum at RT for 60 min. Slides are washed in PBS supplemented with 0.1% Tween-20 (twice, 3 min.) and binding of rabbit antibodies specific for Axl are detected with undiluted Bright Vision poly-HRP-anti-rabbit IgG. HRP is visualized with 3-amino-9-ethylcarbazole (AEC) chromophore (red color; Sigma, cat. no. A6926-100TAB); nuclei were counterstained with hematoxylin (DAKO, cat. no. S3309). Slides are analyzed by a certified pathologist, who score the intensity and localization of Axl staining in each sample.

### Example 2—Anti-Tumor Activity of a Mouse Crossreactive AXL-ADC in an Axl-Expressing Syngeneic Mouse Tumor Model

The Axl antibody YW327.652 (Ye et al., 2010 (b)), which is cross-reactive with mouse Axl, is conjugated with vcMMAE according to the method described previously (WO 2016/005593). The in vivo anti-tumor activity of this mouse crossreactive AXL-ADC is determined in a B16-F10 syngeneic mouse tumor model after prior treatment with PD1 or PD-L1 blocking antibodies. B16-F10 cells (ATCC, cat no. CRL-6475) are transfected with full length mouse Axl, and stably Axl-expressing B16-F10-AXL cells are selected and expanded.

Tumor induction is performed by subcutaneous injection of 1×10⁵ B16-F10 wild type cells or B16-F10-AXL cells into the right flank of female C57Bl/6 mice. Treatment is started when the average tumor size was >100-200 mm³ and distinct tumor growth is observed. Two times per week (every 3-4 days), mice receive intraperitoneal injections with 5 mg/kg anti-mouse PD-1 (Bio X Cell, West Lebanon, N.H.; Clone RMP1-14; Cat no. BP0146) or 5 mg/kg anti-mouse PD-L1 (Bio X Cell; clone 10F.9G2; Cat no. BP0101) until progression of tumor growth is observed. Subsequently, mice intravenously or intraperitonellay receive a single dose or a total of 4 doses in 2 weeks (every 3-4 days) of mouse crossreactive AXL-ADC (4 and 8 mg/kg), control ADC (IgG1-b12-MMAE, 8 mg/kg) or control antibody (unconjugated IgG1-b12, 8 mg/kg), as indicated. Tumor volume is determined at least two times per week. Tumor volumes (mm³) are calculated from caliper (PLEXX) measurements as: 0.52×(length)×(width)².

### Example 3—Antibody Production

AXL-specific antibody IgG1-AXL-107 (WO 2016/005593) and isotype control antibody IgG1-b12 (Barbas, C F. J Mol Biol. 1993 Apr. 5; 230(3):812-23) were expressed as IgG1,κ. Plasmid DNA mixtures encoding heavy and light chains of antibodies were transiently transfected to Expi293F cells (Life technologies, USA) using 293 fectin (Life technologies) essentially as described by Vink et al. (Vink et al., Methods, 65 (1), 5-10 2014). Antibodies were purified by immobilized protein G chromatography. Protein batches were analyzed by a number of bioanalytical assays including SDS-PAGE, size exclusion chromatography and measurement of endotoxin levels. Purified antibodies were conjugated with maleimidocaproyl-valine-citrulline-p-aminobenzoyloxycarbonyl-monomethyl auristatin E (vcMMAE) containing a protease-cleavable valine-citrulline dipeptide as described (Doronina, S. O. et al. (2003) Nat. Biotechnol. 21, 778-784). The average drug-antibody ratio was 4:1. The anti-PD1 antibody pembrolizumab (KEYTRUDA®, MSD) was commercially obtained from SelleckChem (Cat. No.: A2005).

### Example 4—Isolation and Generation of Human, MART-1-Specific CD8 T Cells

MART-1 (1D3) T cell receptor (TCR) retrovirus was produced in a packaging cell line as described previously (Jorritsma et al. (2007) Blood; 110, 3564-3572). Peripheral blood mononuclear cells were isolated from healthy donor buffycoats (Sanquin, Amsterdam, the Netherlands) by density gradient centrifugation using Lymphoprep (Stem Cell Technologies). CD8+ T cells were purified using CD8 Dynabeads (Thermo Fisher Scientific), activated for 48 hours on a non-tissue culture treated 24-well plate that was pre-coated overnight with αCD3 and αCD28 antibodies (eBioscience, 16-0037-85 and 16-0289-85, respectively) at 2×106 per well. Activated CD8 T cells were harvested and mixed with TCR retrovirus (MART-1 T cells) or mock retrovirus (control T cells) and spinfected on a Retronectin coated (Takara, 25 μg per well) non-tissue culture treated 24-well plate for 2 hours at 2000×g. After 24 hours, T cells were harvested and maintained in RPMI (Gibco) containing 10% human serum (One Lamda), 100 units per mL of penicillin, 100 μg per mL of streptomycin, 100 units per mL IL-2 (Proleukin, Novartis), 10 ng per mL IL-7 (ImmunoTools) and 10 ng per mL IL-15 (ImmunoTools).

### Example 5—Anti-Tumor Activity of IgG1-AXL-107-vcMMAE in the SkMel-147 Melanoma Xenograft Model in Mice that is Resistant to Anti-PD-1 Treatment

The anti-tumor activity of IgG1-AXL-107-vcMMAE (HuMax®-AXL-ADC) versus anti-PD-1 (pembrolizumab) was evaluated in the SkMel-147 human melanoma xenograft model in mice that systemically received human T-cells that were engineered to express a melanoma-specific T-cell receptor (TCR) against MART-1. Before inoculation of mice with SkMel-147 cells, the cells were transduced with the antigen (MART-1) as well as the correct HLA haplotype (HLA-A2) in order for the MART-1-specific T cells to recognize the tumor cells.

### Cell Line and Cell Culture Conditions

Melanoma cell line SkMel-147 was cultured in DMEM (Gibco), with fetal bovine serum (Sigma), 100 U/mL penicillin (Gibco) and 0.1 mg/mL streptomycin (Gibco) under standard conditions, and was regularly confirmed to be mycoplasma-free by PCR.

### HLA-A2 and MART-1 Transduction in SkMel-147

MART-126-35 and HLA-A2 were introduced using lentiviral and retroviral constructs. Constructs for lentivirus were packaged in lentivirus using two helper plasmids (psPax and MS2G, Addgene) in HEK293T cells. Constructs for retrovirus were produced in a packaging cell line (Fly cells). Viral supernatant was either snap frozen or immediately used for infection. MART-126-35-Katushka and HLA-A2-GFP double positive cells were sorted by flow cytometry and seeded into 96-well plates at one cell per well. When single cells grew out, expression of HLA-A2 and MART-Katushka were confirmed by FACS.

### SkMel-147 Xenograft Model and Treatment

8-14 week old male and female NOD-SCID Gamma (NSG) mice (bred in-house at the Netherlands Cancer Institute (NKI), Amsterdam, The Netherlands) were subcutaneously injected in the right flank with 1×106 SkMel-147 tumor cells. Tumors were measured three times weekly with a caliper, and when tumors were 50 mm3 (after 9 days) the animals were randomized over the following treatment groups:
1. Control T cells+Control ADC (n=9)
2. MART-1 T cells+Control ADC (n=10)
3. Control T cells+IgG1-AXL-107-vcMMAE (n=10)
4. MART-1 T cells+IgG1-AXL-107-vcMMAE (n=10)
5. MART-1 T cells+Control ADC+anti-PD1 (n=9)

On day 9, mice were i.v. injected with a single dose (2 mg/kg) of IgG1-AXL-107-vcMMAE or control ADC (IgG1-b12-vcMMAE). Simultaneously, mice were i.v. injected with MART-1 or control T cells at a dose of 5×106 cells/mouse. The total injected volume was diluted to 200 μL per mouse in PBS. To support the T cells, all mice received intraperitoneally (i.p.) injection with 100.000 IU IL-2 (Proleukin, Novartis; diluted in 100 μL PBS) for 3 consecutive days.

One selected group (group 5), anti-PD1 (pembrolizumab, SelleckChem) was given weekly via i.p. injection from day 9 onwards, at a dose of 5 mg/kg.

Tumor volumes were measured 3 times weekly by an independent animal technician in a blinded fashion. Tumor volume was calculated as follows: length (mm)×width (mm)/2. Tumors were harvested when they reached 1000 mm3.

### SkMel-147 Sequential Treatment

For selected groups (Control T cells+Control ADC, MART-1 T cells+Control ADC, MART+1 T cells+Control ADC+anti-PD1), a subset of mice were sequentially treated with IgG1-AXL-107-vcMMAE. Mice were selected for sequentially treatment based on a similar tumor volume of ^{˜}650 mm3. IgG1-AXL-107-vcMMAE was weekly i.v. injected at a dose of 4 mg/kg.

### Results

The anti-tumor effects of IgG1-AXL-107-vcMMAE versus anti-PD1 (pembrolizumab) in the SKMel-147 human xenograft mouse model were assessed in the context of a tumor-specific human T-cell response. Therefore, the AXL-expressing human melanoma cell line SkMel-147 was first transduced with both an antigen (MART-1) and the correct HLA haplotype (HLA-A2) in order for tumor-specific T cells to recognize the tumor cells. Subsequently, mice were inoculated with these cells, and after establishment of the xenograft, mice were randomized into different treatment groups (see above), and injected with a single dose of ADC and T cells, while one selected group received additional weekly injections of anti-PD1.

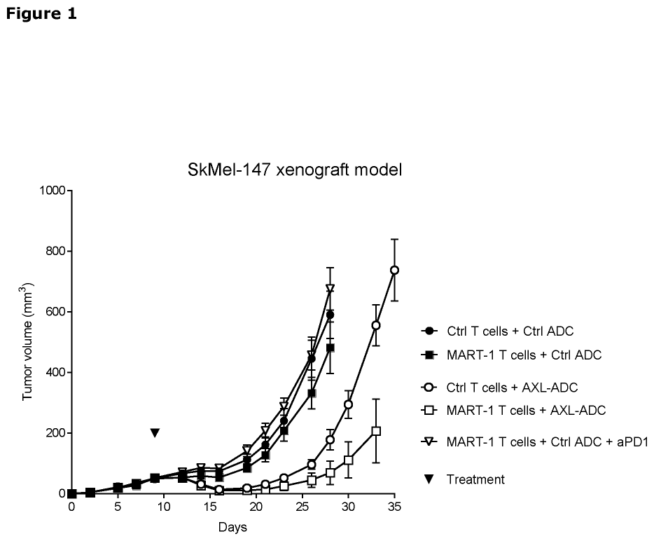

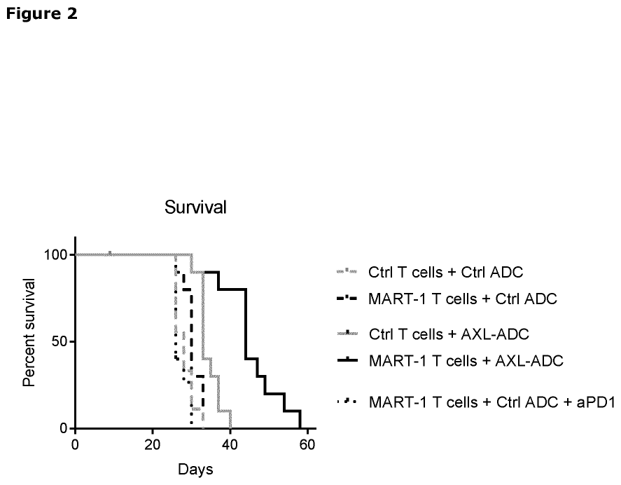

Mice that received tumor antigen-specific T cells (MART-1 T cells) in combination with control ADC showed no differential effect in terms of tumor growth compared to mice that received control, non-specific T cells (Ctrl T cells) in combination with control ADC (FIG. 1). Furthermore, no tumor control was noted in mice that received anti-PD1 treatment in combination with antigen-specific T cells (MART-1 T cells) and control ADC, indicating that this model is resistant to PD-1/PDL-1 axis inhibition (FIG. 1). In comparison, treatment with IgG1-AXL-107-vcMMAE induced tumor regression after a single dose of 2 mg/kg. This effect was observed in mice that received control T cells, and was further enhanced in the setting of MART-1 T cells. IgG1-AXL-107-vcMMAE treatment in the context of MART-1 T cells also prolonged the lifespan of these mice compared to all other groups, as indicated by the survival curve (FIG. 2).

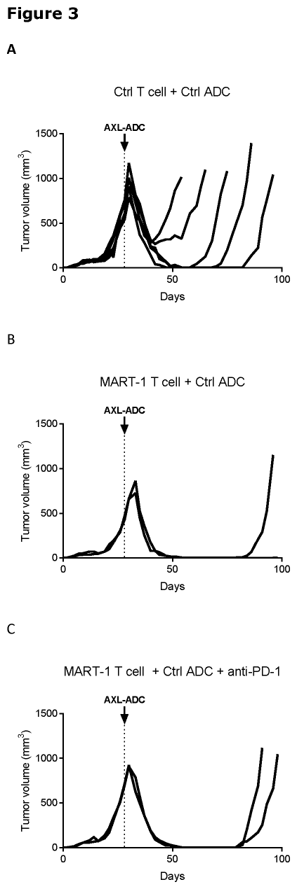

Next, when the average tumor size reached ^{˜}650 mm3 about half of the mice from group 1 (Ctrl T cells+Ctrl ADC), group 2 (MART-1 T cells+Ctrl ADC), and group 5 (MART+1 T cells+Ctrl ADC+anti-PD1) were sequentially treated with IgG1-AXL-107-vcMMAE at a dose of 4 mg/kg in weekly i.v. injections. Whereas the tumors that received no additional treatment quickly reached maximum tumor volume, the IgG1-AXL-107-vcMMAE treated mice showed strong tumor regressions, with tumor volume shrinkage from around 900 mm3 to less than 100 mm3 in two weeks (FIG. 3).

This shows that IgG1-AXL-107-vcMMAE induces anti-tumor effects and survival benefit in the SkMel-147 human melanoma model, which is resistant to PD-1 pathway inhibition in the context of tumor-specific T cells. While PD-1 blockade in the presence of tumor-specific T cells did not affect the tumor growth and survival in this model, IgG1-AXL-107-vcMMAE demonstrated potent anti-tumor and survival effects in the presence of tumor-specific T cells. These results also show that sequential treatment with IgG1-AXL-107-vcMMAE can provide benefit as a single agent in anti-PD-1 resistant tumors in the presence of tumor-specific T cells, indicating that IgG1-AXL-107-vcMMAE can be efficacious in tumors that have progressed on PD-1 inhibitor treatment.

### Example 6—Anti-Tumor Activity of IgG1-AXL-107-vcMMAE in the BLM Melanoma Xenograft Model that is Resistant to Anti-PD-1 Treatment

The anti-tumor activity of IgG1-AXL-107-vcMMAE versus anti-PD1 (pembrolizumab) was evaluated in the BLM human melanoma xenograft model in mice that systemically received human T-cells that were engineered to express a melanoma-specific T-cell receptor (TCR) against MART-1. Before inoculation of mice with BLM cells, the cells were transduced with the antigen (MART-1) as well as the correct HLA haplotype (HLA-A2) in order for the MART-1-specific T cells to recognize the tumor cells.

### Cell Line and Cell Culture Conditions

Melanoma cell line BLM was cultured in DMEM (Gibco), with fetal bovine serum (Sigma), 100 U/mL penicillin (Gibco) and 0.1 mg/mL streptomycin (Gibco) under standard conditions, and was regularly confirmed to be mycoplasma-free by PCR.

### HLA-A2 and MART-1 Transduction in BLM

MART-126-35 and HLA-A2 were introduced using lentiviral and retroviral constructs. Constructs for lentivirus were packaged in lentivirus using two helper plasmids (psPax and MS2G, Addgene) in HEK293T cells. Constructs for retrovirus were produced in a packaging cell line (Fly cells). Viral supernatant was either snap frozen or immediately used for infection. MART-126-35-Katushka positive cells were sorted by flow cytometry and seeded into 96-well plates at one cell per well. When single cells grew out, expression of MART-Katushka and HLA-A2 was confirmed by FACS.

### BLM Xenograft Model and Treatment

8-14 week old male and female NOD-SCID Gamma (NSG) mice (bred in-house at the Netherlands Cancer Institute (NKI), Amsterdam, The Netherlands) were subcutaneously injected in the right flank with 1×10⁶ BLM tumor cells. Tumors were measured three times weekly with a caliper, and when tumors were 100 mm³ (after 7 days) the animals were randomized over the following treatment groups:
1. Control T cells+Control ADC (n=7)
2. MART-1 T cells+Control ADC (n=8)
3. Control T cells+IgG1-AXL-107-vcMMAE (n=8)
4. MART-1 T cells+IgG1-AXL-107-vcMMAE (n=8)
5. MART-1 T cells+Control ADC+anti-PD1 (n=10)

On day 7, mice were i.v. injected with a single dose (4 mg/kg) of IgG1-AXL-107-vcMMAE or control ADC (IgG1-b12-vcMMAE). Simultaneously, mice were i.v. injected with MART-1 or control T cells at a dose of 5×10⁶ cells/mouse. The total injected volume was diluted to 200 μL per mouse in PBS. To support the T cells, all mice received intraperitoneally (i.p.) injection with 100.000 IU IL-2 (Proleukin, Novartis; diluted in 100 μL PBS) for 3 consecutive days.

One selected group (group 5), anti-PD1 (pembrolizumab, SelleckChem) was given weekly via i.p. injection from day 7 onwards, at a dose of 5 mg/kg.

Tumor volumes were measured 3 times weekly by an independent animal technician in a blinded fashion. Tumor volume was calculated as follows: length (mm)×width (mm)/2. Tumors were harvested when they reached 1000 mm3.

### Results

The anti-tumor effects of IgG1-AXL-107-vcMMAE versus anti-PD-1 (pembrolizumab) in the BLM human xenograft mouse model were assessed in the context of a tumor-specific human T cell response. Therefore, the human melanoma cell line BLM was first transduced with an antigen (MART-1) and the correct HLA haplotype (HLA-A2) in order for tumor-specific T cells to recognize the tumor cells. Subsequently, mice were inoculated with these cells, and after establishment of the xenograft, mice were randomized into different treatment groups (see above), and injected with a single dose of ADC and T cells, while one selected group received additional weekly injections of anti-PD1.

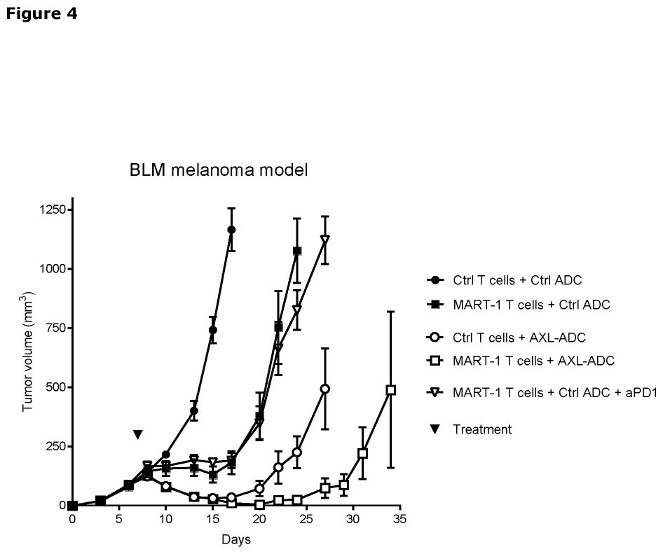

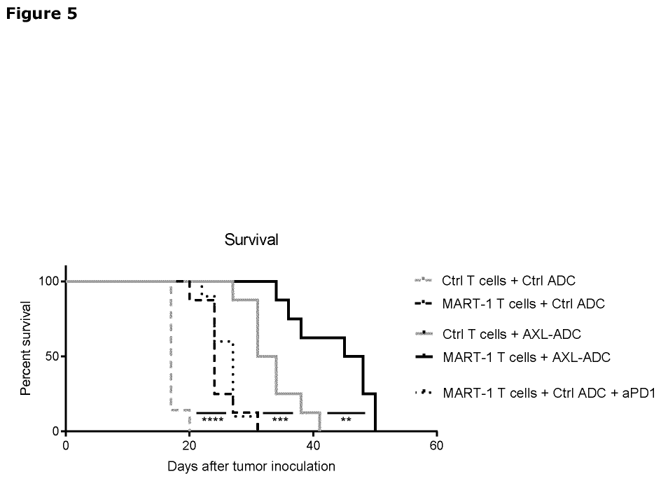

Mice that received antigen-specific T cells (MART-1 T cells) in combination with control ADC showed some tumor growth inhibition compared to mice that received control, non-specific T cells (Ctrl T cells) in combination with control ADC (FIG. 4). However, no enhanced tumor growth inhibition was noted in mice that received anti-PD1 treatment in combination with antigen-specific T cells (MART-1 T cells) and control ADC, indicating that this model is resistant to PD-1/PDL-1 axis inhibition (FIG. 4). In comparison, treatment with IgG1-AXL-107-vcMMAE induced tumor regression after a single dose of 4 mg/kg. This effect was observed in mice that received control T cells, and was further enhanced in the setting of MART-1 T cells. In both instances, treatment with IgG1-AXL-107-vcMMAE led to greater anti-tumor effects compared to tumor-specific T cells alone or in combination with anti-PD1. IgG1-AXL-107-vcMMAE treatment in the context of MART-1 T cells also prolonged the lifespan of these mice compared to all other groups, as indicated by the survival curve (FIG. 5).

These results show that IgG1-AXL-107-vcMMAE treatment is efficacious in the BLM human melanoma model which is resistant to anti-PD1 treatment in the setting of tumor-specific T cells. While inhibition of PD-1 in the presence of tumor-specific T cells had no effect on tumor growth and survival, IgG1-AXL-107-vcMMAE led to potent tumor reduction and survival benefit, consistent with efficacy in tumors resistant to PD-1/PDL-1 axis blockade.

### Example 7—First-in-Human, Open-Label, Dose-Escalation Trial with Expansion Cohorts to Evaluate Safety of Axl-Specific Antibody-Drug Conjugate (HuMax®-AXL-ADC; Enapotamab Vedotin) in Patients with Solid Tumors

The present study was an open label, multi-center Phase I/IIa safety trial of HuMax AXL ADC in a mixed population of patients with solid tumors known from the literature to overexpress Axl and where the use of systemic tubulin inhibitors was part of Standard of Care (SoC). The trial consisted of two parts; a dose escalation part (phase I, first-in-human (FIH)) and an expansion part (phase IIa).

The dose escalation part consists of two, staggered, arms for identification of the most optimal dosing regimen:
1Q3W: Dosing once every 3 weeks
3Q4W: Weekly dosing for 3 weeks followed by one treatment-free week.

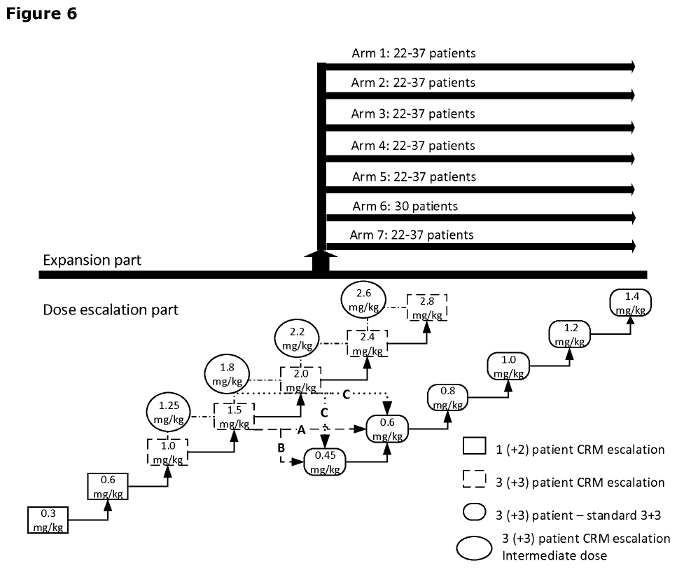

The aim of the expansion part of the study was to provide further data on the safety, tolerability, PK and anti-tumor activity of the selected dose. The overall design of the study is presented in FIG. 6.

### Inclusion Criteria:

Patients had to meet all of the following inclusion criteria before they were allowed to participate in the trial:
1. For the dose escalation part: Patients with relapsed or refractory cancer of the ovary, cervix, endometrium, thyroid, non-small cell lung cancer (NSCLC), or melanoma (cutaneous, mucosal, acral or uveal melanoma) who had failed available standard therapy or who are not candidates for standard therapy, and for whom, in the opinion of the investigator, experimental therapy with HuMax-AXL-ADC would possibly be beneficial.
2. For the expansion part: Patients with relapsed or refractory, advanced and/or metastatic cancer who were not candidates for standard therapy, and for whom, in the opinion of the investigator, experimental therapy with HuMax-AXL-ADC could be beneficial.

Expansion cohorts included patients with solid tumors, for instance non-small cell lung cancer (NSCLC), an melanoma (including cutaneous, acral, and mucosal melanoma). The patients were included on the basis of the following criteria:
Documented progressive disease on or after last prior treatment
Last treatment prior to enrollment was treatment with a PD-1/PD-L1 inhibitor

For the following condition in the Expansion Cohorts, the sponsor medical officer's approval of enrolment was needed:
if documented progression had not been on measurable disease (i.e. symptomatic progression).

Patients were required to have measurable disease according to RECIST (Response Evaluation Criteria In Solid Tumors) version 1.1.
A minimum of one lesion ≥10 mm (or twice the slice thickness if slices were not 5 mm thick) in the longest diameter (LD) from a non-irradiated area
   Lymph nodes lesion ≥15 mm in the shortest diameter from a non-irradiated area.
   If target lesion(s) were located within previously irradiated area patients could be enrolled if:
      target lesions had not been irradiated within the last 3 months.
      there had been demonstrated progression in the “in field” target lesion and after sponsor acceptance

In the dose escalation part, patients with ovarian cancer could be included based on CA 125 positivity according to the Gynecologic Cancer Intergroup Guideline (Rustin et al., 2004; Rustin et al., 2011); only if they had a pretreatment sample that was at least twice the upper limit of the reference range and within 2 weeks before starting the treatment.

Patients were not evaluable by CA 125 if they had received mouse antibodies (unless the assay used had been shown not to be influenced by human anti-mouse antibody) or if there had been medical and/or surgical interference with their peritoneum or pleura during the previous 28 days (e.g. paracentesis).

In the dose escalation part, all patients were required to provide a tumor tissue sample (Formalin Fixed Paraffin Embedded (FFPE) blocks/slides) from archival tissue or fresh biopsy collected before Cycle 1, Visit 1, preferably derived from advanced disease stage.

In the expansion part, all patients were required to provide a mandatory fresh biopsy (FFPE tissue blocks/slides) at screening (aspirates are not acceptable) which contained tumor tissue and was taken after failure/stop of last prior treatment, unless not clinically feasible as documented by investigator.

Documentation of the fresh FFPE biopsy shipment had to be submitted to the Sponsor as a part of eligibility package prior to administration of first dose of enapotamab vedotin. In case it was not feasible to meet the required criteria for fresh tumor biopsy, the sponsor medical officer's approval of enrollment was needed. Furthermore, the latest available archival tumor tissue sample, which was taken before failure/stop of last prior treatment, had to be be collected if available.

Age ≥18 years.

Have an acceptable renal function defined as:
Glomerular filtration rate (GFR) ≥40 mL/min/1.73 m²—e.g., according to the abbreviated Modification of Diet in Renal Disease (MDRD) equation:

GFR=186×(SCr^{-1.154})×(age^{-0.203})
(where SCr, the serum creatinine level, is expressed in mg/dL; multiply it by 0.742 if the patient is female; multiply it by 1.212, if the patient is African-American).
Not being on dialysis

Have an acceptable liver function defined as:
Alanine aminotransferase (ALT) and aspartate aminotransferase (AST) ≤3 times the upper limit of normal (ULN); if liver tumor/metastases were present, then ≤5×ULN was allowed.
Bilirubin ≤1.5×ULN, except in patients diagnosed with Gilbert's syndrome, direct bilirubin ≤2×ULN

Have an acceptable hematological status defined as:
Hemoglobin 5.6 mmol/L (^{˜}9 g/dL).
Absolute neutrophil count (ANC) ≥1500/μL (1.5×109/L).
Platelet count ≥100×109/L.

Have an Eastern Cooperative Oncology Group (ECOG) performance status of 0 or 1.

Life expectancy of at least 3 months.

Patients, both females and males, of childbearing/reproductive potential had to agree to use adequate contraception while included in the trial and for 6 months after the last infusion of Enapotamab vedotin.

Patients were required to provide signed informed consent form (ICF).

### Exclusion Criteria

If any of the following applied, the patient was required to not enter the trial:

### Hematological

1. Acute deep vein thrombosis or clinically relevant pulmonary embolism, not stable for at least 4 weeks prior to first enapotamab vedotin administration.
2. Patient having a history of thromboembolic event(s) and not being willing to take thromboembolic prophylaxis.

### Cardiovascular

3. Have clinically significant cardiac disease, including:
Onset of unstable angina within 6 months of signing the ICE.
Acute myocardial infarction within 6 months of the signing the ICE.
Known congestive heart failure (Grade III or IV as classified by the New York Heart

Association); and/or a known decreased cardiac ejection fraction of <45% and/or baseline QT interval as corrected by Fridericia's formula (QTcF) >480 msec or uncontrolled atrial fibrillation.
Uncontrolled hypertension defined as systolic blood pressure ≥160 mmHg and/or diastolic blood pressure ≥100 mmHg, despite optimal medical management.

### Immunological

4. Ongoing or recent (within 1 year) evidence of significant autoimmune disease that required treatment with systemic immunosuppressive treatments, which could suggest risk for immune related adverse events.
5. Patients with a history of Grade 3 or higher immune related adverse events were excluded (adverse events below Grade 3 had to be discussed with the sponsor).
6. Patients with ongoing pneumonitis at screening or with a history of non-infections pneumonitis that required steroids.

### Excluded Medications or Treatment Regimens

7. Have received granulocyte colony stimulating factor (G-CSF) or granulocyte/macrophage colony stimulating factor support 3 weeks prior to first enapotamab vedotin administration.
8. Have received a cumulative dose of corticosteroid >150 mg prednisone (or equivalent doses of corticosteroids) within two weeks before the first enapotamab vedotin administration.
9. History of ≥Grade 3 allergic reactions to monoclonal antibody therapy as well as known or suspected allergy or intolerance to any agent given in the course of this trial.

### Surgery/Procedures

10. Major surgery within 4 weeks before first Enapotamab vedotin administration.

### Central Nervous System

11. Any history of intracerebral arteriovenous malformation, cerebral aneurysm, brain metastases or stroke.
Transient ischemic attack 6 months prior to screening was allowed.
Patients with known or suspected central nervous system metastases symptoms were required to undergo a Computed Tomography (CT) scan or Magnetic Resonance Imaging of the brain and/or spinal cord for documentation of baseline disease status. Spinal cord metastasis was acceptable. However, patients with known spinal cord compression that was symptomatic or patients who had not undergone definitive treatment for the spinal cord compression and subsequently did not have evidence of clinically stable disease (SD) for at least 28 days, were excluded.
In the expansion cohorts the enrolment of patients with stable brain metastases, i.e., being asymptomatic for the last 14 days prior to treatment initiation, was allowed.
Symptomatic uncontrolled brain or leptomeningeal metastases. (To be considered “controlled”, central nervous system [CNS] disease was required to have undergone treatment (e.g., radiation or chemotherapy) at least 2 weeks prior to first enapotamab vedotin administration. The patient could not have any new or progressive signs or symptoms related to the CNS disease and must be taking <10 mg of prednisone or equivalent per day or no steroids). Patients who had untreated brain metastases and who were not symptomatic were allowed to enroll if the investigator felt that treatment of these metastases was not indicated. Patients with spinal cord compression could be considered for enrolment if they had received definitive treatment for this and evidence of clinically stable disease (SD) for at least 28 days.

### Prior Therapy

12. Any anticancer therapy including; small molecules, immunotherapy, chemotherapy monoclonal antibodies or any other experimental drug within 5 half-lives but maximum 4 weeks before first infusion. Accepted exceptions were bisphosphonates, denosumab and gonadotropin-releasing hormone agonist or antagonist, which could be continued throughout the trial.
Toxic effects of prior anti-cancer therapy considered as chronic, such as chemotherapy-induced fatigue, alopecia, or anorexia of Grade 2, where no more resolution was expected, did not prevent the patient from participation in the trial.
   13. Any prior therapy with a conjugated or unconjugated auristatin derivative/vinca-binding site targeting payload. (Previous treatment with vinca alkaloids was allowed in line with inclusion criterion #1.)
   14. Radiotherapy within 14 days prior to first enapotamab vedotin administration. (Palliative radiotherapy was allowed.

### Other Cancer/Metastases

15. Known past or current malignancy other than inclusion diagnosis, except for:
Cervical carcinoma of Stage 1B or less.
Non-invasive basal cell or squamous cell skin carcinoma.
Non-invasive, superficial bladder cancer.
Prostate cancer with a current PSA level <0.1 ng/mL.
Breast cancer in BRCA1 or BRCA2 positive ovarian cancer patients.
Any curable cancer with a complete response (CR) of >2 years duration.

### Other

16. Melanoma patients with an LDH ≥3×ULN.
17. Ongoing significant, uncontrolled medical condition including:
Serious, non-healing wound, skin ulcer (of any grade), or bone fracture.
   18. Presence of ≥Grade 2 peripheral neuropathy.
   19. Clinically significant active viral, bacterial or fungal infection requiring:
I.v. treatment with anti-infective therapy that had been administered less than two weeks prior to first dose, or
Oral treatment with anti-infective therapy that had been administered less than one week prior to first dose.
Prophylactic anti-infective therapy, which was given without clinical symptomatic was allowed (e.g. antibiotic prophylaxis prior to dental extraction, etc.).
   20. Known human immunodeficiency virus seropositivity.
   21. Known history/positive serology for hepatitis B (unless immune due to vaccination or resolved natural infection or unless passive immunization due to immunoglobulin therapy):
Positive test for antibodies to hepatitis B core antigens (anti-HBc); and
Negative test for antibodies to hepatitis B surface antigens (anti-HBs).
   22. Known positive serology for hepatitis C (unless due to immunoglobulin therapy)
   23. Substance abuse, medical, psychological or social conditions that could interfere with the patient's participation in the trial or evaluation of the trial result
   24. History of organ allograft (except for corneal transplant) or autologous or allogeneic bone marrow transplant, or stem cell rescue within 3 months prior to the first dose of enapotamab vedotin
   25. Body weight <40 kg
   26. Women who are pregnant or breast feeding.
   27. Patients are not allowed to take part in any other interventional trial while participating in current trial.

### Specifically for NSCLC

28. Pulmonary hemorrhage or hemoptysis >2.5 ml blood within 6 weeks unless cause has been addressed and is medically resolved.
29. History of acute pneumonitis.

### Dose Escalation and Mode of Administration:

### 1Q3W

The 1Q3W dose escalation evaluated HuMax-AXL-ADC at seven main dose levels: 0.3, 0.6, 1.0, 1.5, 2.0, 2.4 and 2.8 mg/kg, and 4 optional intermediate dose levels 1.25, 1.8, 2.2 and 2.6 mg/kg. Further escalation with steps of 0.4 mg/kg and de-escalation by 0.2 mg/kg was allowed, if the MTD had not been declared at a dose level up to 2.8 mg/kg.

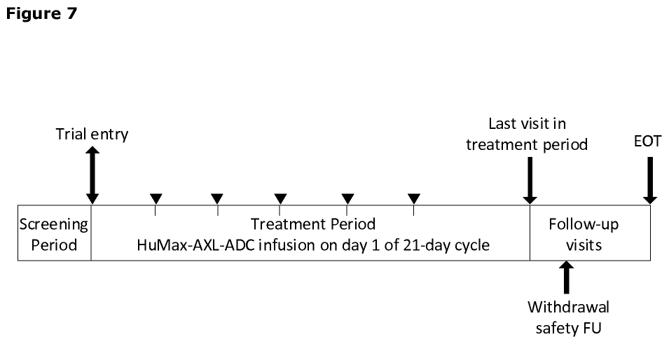

In the 1Q3W dose escalation the patients received 1 infusion of HuMax-AXL-ADC every three weeks as according to FIG. 7.

### 3Q4W

When a minimum of 8 patients had been treated and evaluated for Dose limiting toxicities (DLTs), the 1.5 mg/kg cohort was declared safe on the 1Q3W arm, and the predicted AUC on the starting dose in 3Q4W arm was below pre-defined limits, the 3Q4W arm was initiated.

The 3Q4W dose escalation was conducted as a standard 3 (+3) design which evaluated HuMax-AXL-ADC at doses of (0.45), 0.6, 0.8, 1.0, 1.2 and 1.4 mg/kg. The escalation was allowed to continue to higher dose levels with increments up to 20%, if the 1.4 mg/kg was reached without significant safety concerns and it was considered safe to escalate above 1.4 mg/kg, the. The starting dose was expected to be 0.6 mg/kg (a dose level of 0.45 mg/kg could be added) and as an additional precaution, the independent Data Monitoring Committee (DMC) could recommend intermediate dose levels at any step during dose escalation.

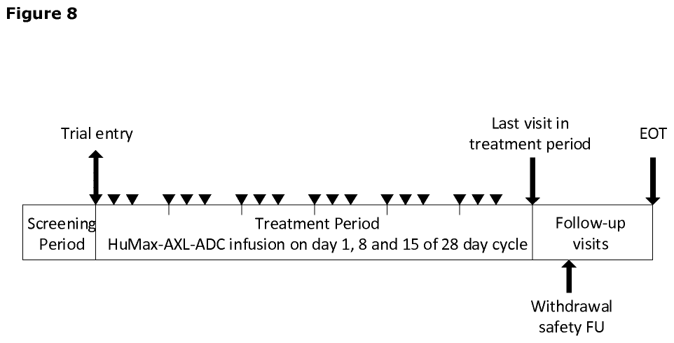

In the 3Q4W dose escalation the patients received weekly dosing for 3 weeks followed by one treatment-free week according to FIG. 8. Patients were treated until disease progression or unacceptable toxicity was observed.

### Rationale for Dose Frequency

In the dose escalation part, HuMax-AXL-ADC was administered 1Q3W in the first dose escalation arm and 3Q4W in the second dose escalation arm. The dosing frequency was based on toxicokinetic and toxicology data obtained in cynomolgus monkeys, suggesting adequate recovery of neutrophils, thrombocytes and red blood cell parameters and otherwise an acceptable safety profile. No relevant accumulation of HuMax-AXL-ADC or MMAE between cycles was anticipated.

### Treatment Preparation

The dose of HuMax-AXL-ADC for administration was prepared by the site pharmacy using aseptic technique. HuMax-AXL-ADC was supplied to the site/pharmacy as bulk supply cartons. Labelling of the IMP was done in accordance with local standards and regulations.

The Investigational Medicinal Product (IMP) was supplied in vials containing 40 mg of HuMax-AXL-ADC as lyophilized powder. The powder was reconstituted with 4 mL water for injection leading to a 10 mg/mL solution.

The reconstituted HuMax-AXL-ADC was diluted into 0.9% NaCl 100 mL infusion bag according to the dose assigned to the patient.

HuMax-AXL-ADC (lyophilized vials) were stored in a refrigerator at 2° C. to 8° C.

The infusion was required to be completed within 24 hours after the HuMax-AXL-ADC vials have been reconstituted. An in-line filter 0.2 μm must be used for the infusion. The entire 100 mL infusion volume from the prepared infusion bag needs to be administered, no dead volume is provided.

### Treatment Administration

HuMax-AXL-ADC was administered as an intravenous infusion. Each patient's dose was calculated based on the patient's weight rounded to the nearest kilogram, i.e., assigned cohort dose in mg/kg×body weight in kg. For patients whose body mass index (BMI) was greater than 30 kg/m², the investigator was required to use a weight that, based on the patient's height, corresponds to a maximum BMI of 30.

The dose was calculated according to the following formula if BMI was greater than 30 kg/m²:

Dose (mg)=x (mg/kg)*30 (kg/m2)*height (m)*height (m) (where x denotes the dose level)

HuMax-AXL-ADC was administered over a minimum of 30 minutes and the infusion must be completed within 4 hours. The infusion was complete when the infusion line had been flushed with saline.

In the dose-escalation part, there was a minimum of 2 nights between the first and second patient in each dose cohort to account for any safety concerns in each new dose.

### Duration of Treatment:

Dependent on which dose-escalation arm the patient was recruited to, HuMax AXL ADC was administered either 1Q3W or 3Q4W. The patients received treatment with HuMax-AXL-ADC until disease progression or unacceptable toxicity. Patients were followed for 52 weeks after end of treatment. In the expansion part of the trial patients received HuMax AXL ADC at the maximum tolerated dose (MTD) found in either 1Q3W or 3Q4W schedule as recommended by the DMC and confirmed by the internal sponsor safety committee.

### Criteria for Evaluation:

### Primary Endpoints

Dose Limiting Toxicities (DLTs)
Adverse events (AEs): incidences of AEs, serious adverse events (SAEs), infusion-related AEs, ≥grade 3 AEs, and AEs related to IMP during the trial.

### Secondary Endpoints

Safety laboratory parameters (hematology and biochemistry).
PK parameters (clearance, volume of distribution and area-under-the-concentration-time curve [AUC_{0-Clast} and AUC_{0-∞}], maximum concentration [Cₘₐₓ], time of Cₘₐₓ [Tₘₐₓ], pre dose values, and half-life of HuMax-AXL-ADC and free toxin monomethyl auristatin E [MMAE]).
Immunogenicity of HuMax-AXL-ADC (anti-drug antibodies).
Anti-tumor activity measured by tumor shrinkage (based on computerized tomography [CT]-scan evaluations), as well as change in CA 125 in patients with ovarian cancer and change in prostate specific antigen (PSA) in patients with castration-resistant prostate cancer (CRPC).
Objective Response, Progression-Free Survival (PFS), Duration of Response (DoR) and Overall survival (OS).
Axl expression in the tumor biopsy.

### Response

Response in solid tumor cancers was assessed in accordance with the RECIST criteria version 1.1 and for patients with ovarian cancer according to RECIST 1.1 in combination with CA 125 as defined by the Gynecological Cancer Intergroup (Rustin et al., 2011).

TABLE 5
Definition of Response (RECIST Criteria v1.1)

| | Category | Criteria |
|---|---|---|
| Based on | Complete Response | Disappearance of all target lesions. |
| target lesions | (CR) | Any pathological lymph nodes |
| | | must have reduction in short axis |
| | | to <10 mm. |
| | Partial Response | ≥30% decrease in the sum of the |
| | (PR) | LD of target lesions, taking as |
| | | reference the baseline sum LD. |
| | Stable Disease | Neither sufficient shrinkage to |
| | (SD) | qualify for PR nor sufficient |
| | | increase to qualify for PD, taking |
| | | as reference the smallest sum of |
| | | LDs since the treatment started. |
| | Progressive Disease | ≥20% increase in the sum of the |
| | (PD) | LDs of target lesions, taking as |
| | | reference the smallest sum of |
| | | the LDs recorded since the |
| | | treatment started or the |
| | | appearance of one or more |
| | | new lesions. |
| Based on non- | CR | Disappearance of all non-target |
| target lesions | | lesions and normalization of |
| | | tumor marker level. All lymph |
| | | nodes must be non-pathological |
| | | in size (<10 mm short axis). |
| | SD | Persistence of one or more |
| | | non-target lesion(s) or/and |
| | | maintenance of tumor marker |
| | | level above the normal limits. |
| | PD | Appearance of one or more new |
| | | lesions and/or unequivocal |
| | | progression of existing non- |
| | | target lesions. |

### Response Evaluation and Reporting of Results

In the dose escalation, response evaluation was performed by the investigator and sponsor. In the expansion, response evaluation was performed by the investigator and sponsor as well as a group of external medical experts. Each patient was assigned one of the following categories:

### 1) CR,

### 2) PR,

### 3) SD,

### 4) PD, or

### 5) Not Evaluable

Patients in response categories 1 and 2 were considered responders and patients in response categories 4 and 5 were considered as failing to respond to treatment (disease progression). Patients in response categories 1, 2 and 3 were considered to be in disease control.

Individual patient data listings and summaries of objective response, best overall tumor response (based primarily on confirmed but also on unconfirmed response) and disease control was to be presented.

For patients with ovarian cancer, responses were to be evaluated and reported as per RECIST 1.1 (Eisenhauer et al., 2009), CA 125 and the combination of the two sets of response criteria in accordance with the Gynecological Cancer Intergroup definitions (Rustin et al., 2011).

For patients with prostate cancer, responses were to be evaluated and reported as per RECIST 1.1 (Eisenhauer et al., 2009) and PSA according to the Updated Recommendations from the Prostate Cancer Clinical Trials Working Group 3 (Scher et al, 2016).

### Progression-Free Survival

PFS is defined as the number of days from Visit 1 in Cycle 1 to first PD or death. Only deaths that occurred within 30 days of the last progression assessment were to be considered in the analysis. If no death was observed within this period, PFS was to be censored at the last progression assessment. PFS was derived for all patients and presented graphically as well as summarized using survival analysis methods: distribution functions were to be estimated using Kaplan-Meier technique and times were to be censored in accordance with Table A in Appendix 3 in the FDA Guidance for Industry: Clinical Trial Endpoints for the Approval of Cancer Drugs and Biologics (2007).

### Duration of Response

DoR is defined as the number of days from the first documentation of objective tumor response (CR or PR) to the date of first PD or death. DoR was to be analyzed using the same statistical methodology as PFS.

### Overall Survival

Overall survival (OS) is defined as the number of days from Visit 1 in Cycle 1 to death. OS was analyzed using the same statistical methodology as PFS and DoR except that censoring was not applied neither when visits were skipped nor when new anti-cancer therapies were given.

### Tumor Shrinkage

Tumor shrinkage (based on CT-scan evaluations) was listed and summarized, per source (radiologist, central read).

### Results:

### Dose Escalation

Results: 47 patients with NSCLC (n=8), melanoma (n=9), ovarian (n=22), cervical (n=3) and endometrial (n=5) cancer enrolled in Phase 1 (1Q3W n=32; 3Q4W n=15). Most patients were female (87%), White (94%) and aged <65y (66%). Maximum Tolerated Dose (MTD) was 2.2 mg/kg in the 1Q3W arm and 1.0 mg/kg in the 3Q4W arm; Recommended Phase 2 Dose (RP2D) was 2.2 mg/kg for the 1Q3W dosing regime. Enapotamab Vedotin median elimination half-life was 0.9-2.2 days across doses/schedules. In the 47 patients enrolled, there were 6 DLTs (Table). The most common Adverse Effects (any grade; in ≥40% of patients) were fatigue (64%), nausea (57%), constipation (57%), diarrhea (47%), vomiting (45%) and decreased appetite (43%). Three patients in the 1Q3W arm had a partial response (1 NSCLC [2.2 mg/kg dose]; 2 ova2rian [1.5 and 2.4 mg/kg dose levels]).

Conclusions: The RP2D of single agent Enapotamab Vedotin in pre-treated patients with solid tumors was 2.2 mg/kg 1Q3W. Enapotamab Vedotin showed encouraging preliminary antitumor activity.

| DLT | Dose, mg/kg (n) |
|---|---|
| 1Q3W | |
| Constipation | 2 (1); 2.2 (1) |
| Vomiting | 2.2 (1) |
| GGT increase | 2.4 (1) |
| 3Q4W | |
| Febrile neutropenia | 1.2 (1) |
| Diarrhea | 1.2 (1) |

### NSCLC Patients, Subject Examples:

### Subject 401

This 71 year old, white female patient was enrolled in the study GEN1021 and signed the informed consent form on the 11 Apr. 2018 at a site in the UK.

The patient was diagnosed with stage IIIA, non-small cell lung andenocarcinoma (negative for ALK rearrangement) on the 5 Aug. 2016.

Past cancer treatments included cisplatin plus vinorelbin from August to September 2016, reported with progression during treatment and a best response of progressive disease (PD). The patient received cisplatin plus premetrexed from October 2016 to November 2016 with a best response of partial response (PR) but treatment was discontinued due to toxicity. Patient received Erlotinb from June to August 2017 with best response of PD and last prior treatment before enrolment on GEN 1021 was pembrolizumab from September 2018 to January 2018 with a best response of stable disease (SD). Treatment with pembrolizumab was discontinued due to progression of disease.

Medical history included childhood polio and subdural hematoma, both conditions resolved at the time of enrollment. In addition the patient had peripheral neuropathy, cough and right eye cataract, all conditions ongoing at the time of enrollment. Patient is a non-smoker and was reported with an ECOG of 1 at the time of enrollment.

Patient received the first dose of enapotamab vendotin on C1D1 (20 Apr. 2018).

Treatment emerging events include urinary tract infection (G2, unrelated), creatinine kinase increase (fluctuating between G1 and G2, possibly related), muscle cramps (G1, possibly related), worsening of cough (G2, unrelated) and ALT increase and AST increase (both G1 and unrelated). For none of these events study drug administration was altered. In addition patient experienced the event of dysphonia and left leg weakness, both events reported as G1 and possibly related and due to these events, study drug administration was interrupted.

At screening, two target lesions (TLs) were identified in the lungs, one in left lower lobe reported with the longest diameter of 11 mm and one in the right upper lobe reported with the longest diameter of 15 mm. The sum of diameters at screening was 26 mm. In addition, one non-target lesion (NTL) was identified in the lung (site not specified).

At C2D15 (25 May 2018), first post-baseline scan was performed. At that time, TL in the left lower lobe was reported with a diameter of 10 mm and the TL in the upper right lung with a diameter of 12 mm and thus the sum of diameters of 22 mm. As compared to screening, this corresponds to 15% decrease in sum of diameters. The NTL was reported as present (SD) and no new lesions were detected. The overall response assessment was reported as SD according to RECIST 1.1.

At C4D15 (Jun. 7, 2018), the second post-baseline scan was performed. At that time, TL in the left lower lobe was reported with a diameter of 8 mm and the TL in the upper right lung with a diameter of 9 mm and thus the sum of diameters of 17 mm. As compared to screening, this corresponds to 34.6% decrease in sum of diameters. The NTL was reported as present (SD) and no new lesions were detected. The overall response assessment was reported as PR according to RECIST 1.1.

At C6D15 (17 Aug. 2018), the third post-baseline scan was performed. At that time, TL in the left lower lobe was reported with a diameter of 5 mm and the TL in the upper right lung with a diameter of 6 mm and thus the sum of diameters of 11 mm. As compared to screening, this corresponds to 57.6% decrease in sum of diameters. The NTL was reported as present (SD) and no new lesions were detected. The overall response assessment was reported as PR according to RECIST 1.1.

### Subject 403

This 63 year old, white female patient was enrolled in the study GEN1021 and signed the informed consent form on the 4 May 2018 at a site in the UK.

The patient was diagnosed with stage IV, non-small cell lung andenocarcinoma (negative for EGFR mutations and ALK rearrangement) on the 19 Jan. 2017.

Past cancer treatments included carboplatin plus pemetrexed from February 2017 to March 2017, reported with progression during treatment and a best response of PD. The patient was treated with radiotherapy in April 2017, with a best response of PR. Last prior treatment before enrolment on GEN 1021 was pembrolizumab from June 2017 to September 2017 with a best response of PD.

Medical history included cervical intraepithelial neoplasia Dizziness, light headaches and constipation, all of which were resolved at the time of enrollment. Hypertension, neck osteoarthritis, gallstones, postural hypotension, fatigue, cough, intermittent left sided chest pain, anxiety, arthralgia, anorexia and dry skin were reported as ongoing medical conditions at the time of enrollment.

Patient was a past-smoker (47 years) but discontinued smoking in January 2017. Patient was reported with an ECOG of 1 at the time of enrollment.

Patient received the first dose of enapotamab vendotin on C1D1 (15 May 2018).

Treatment emerging events include two episodes of nausea (both Gland possibly related), skin and subcutaneous tissue disorder (G1, not related), constipation (G2, related), two episodes of anorexia (G1, first episode unrelated, second episode possibly related) gastroesophageal reflux (G1, not related), alopecia (G1, related), AST increase (G1, possibly related). For none of the reported events, study treatment administration was changed.

At screening four TLs were identified. The lesions were following: Left axillary nodal mass reported with diameter of 24 mm, right lower lung lobe lesion with diameter of 15 mm, right lower lung lobe lesion with diameter of 13 mm and right iliac lesion with diameter of 36 mm. The sum of diameters at screening was 88 mm. In addition, two NTLs were identified, one in the right middle lobe of the lung and in left supraclavicular fossa lymph node

At C2D15 (19 Jun. 2018), first post-baseline scan was performed. At that time, the left axillary nodal mass reported with diameter of 14 mm, right lower lung lobe lesion with diameter of 12 mm, right lower lung lobe lesion with diameter of 9 mm and right iliac lesion with diameter of 36 mm. The sum of diameters at C2D15 was 71 mm. As compared to screening, this corresponds to 19.3% decrease in sum of diameters. The NTLs were reported as present (SD) and no new lesions were detected. The overall response assessment was reported as SD according to RECIST 1.1.

At C4D15 (31 Jul. 2018), the second post-baseline scan was performed. At that time the left axillary nodal mass reported with diameter of 10 mm, right lower lung lobe lesion with diameter of 9 mm, right lower lung lobe lesion with diameter of 6 mm and right iliac lesion with diameter of 32 mm. The sum of diameters at C2D15 was 57 mm. As compared to screening, this corresponds to 35.2% decrease in sum of diameters. One of the two NTLs was reported as present whereas the other was reported as absent (SD) and no new lesions were detected. The overall response assessment has not been reported in the eCRF to date.

At C6D15 (Nov. 9, 2018), the third post-baseline scan was performed. At that time the left axillary nodal mass reported with diameter of 10 mm, right lower lung lobe lesion with diameter of 9 mm, right lower lung lobe lesion with diameter of 7 mm and right iliac lesion with diameter of 30 mm. The sum of diameters at C2D15 was 56 mm. As compared to screening, this corresponds to 36.4% decrease in sum of diameters. To date, the status of the two NTLs has not been reported in the eCRF and no new lesions were detected. Overall TL assessment had been reported as PR, overall status of NTL has been reported as “not evaluable” but overall response assessment for this time point had not yet been reported.

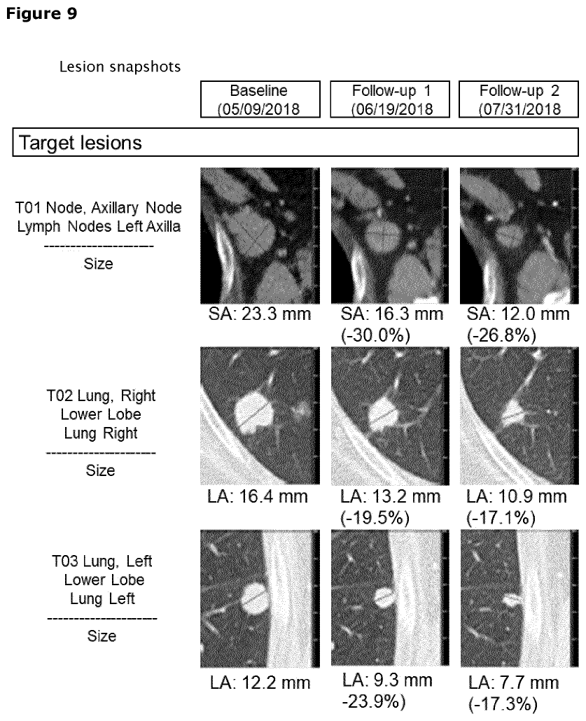

Lesion snapshots are provided in FIG. 9.

### Subject 406

This 64 year old, white male patient was enrolled in the study GEN1021 and signed the informed consent form on the 11 Jun. 2018 at a site in the US.

The patient was diagnosed with stage IV, non-small cell lung andenocarcinoma (negative for EGFR mutations and ALK rearrangement) on the 20 Dec. 2016.

Past cancer treatments included carboplatin plus pemetrexed from December 2016 to February 2017, reported with progression during treatment and a best response of PD. The patient was treated with duruvalumab plus IPH-2201 (anti-NKG2A) from March 2017 to May 2017 with a best response of PD. The patient was subsequently treated with docetaxel plus ramucirumab from May 2017 to September 2017 with a best response of PD. Patient was treated with gemcitabine from October 2017 to January 2018, best response unknown but patient discontinued treatment due to PD. Patient received palliative radiotherapy in March 2018 (response to treatment not reported).

Medical history included hypertension, hyperlipidemia, fatigue, appetite and weight change, shortness of breath, depression and back pain. All conditions were ongoing at the time of enrollment.

Patient was a past-smoker (32 years) but discontinued smoking in January 2004. Patient was reported with an ECOG of 1 at the time of enrollment.

Patient received the first dose of enapotamab vendotin on C1D1 (20 Jun. 2018).

Treatment emerging events include two episodes of back pain (G2 and G3, both unrelated), neutropenia (G3, possibly related), fatigue (G2, not related), hypotension (G3, not related), hyponatremia (G3, not related), puritis (G1, possibly related), dry skin (G1, possibly related), neuropathy (G1, not related), anorexia (G2, not related), insomnia (G1, not related) and weight loss (G2, possibly related). Drug was interrupted due to G3 back pain but administration was not changed to any of the other events.

At screening, two TLs were identified in the lung, a right lung lesion reported with a diameter of 18 mm and a left lung lesion reported with a diameter of 14 mm. The sum of diameters at screening was 32 mm. In addition, one NTL was identified, a bilateral lung lesion.

At C2D15 (Aug. 8, 2018), first post-baseline scan was performed. At that time, the right lung lesion reported with a diameter of 8 mm and a left lung lesion reported with a diameter of 9 mm. The sum of diameters at C2D15 was 17 mm. As compared to screening, this corresponds to 46.8% decrease in sum of diameters. The NTL was reported as present (SD) and no new lesions were detected. The overall response assessment was reported as PR according to RECIST 1.1.

### REFERENCES

Bird et al., Science 242, 423-426 (1988)
Brochet X. Nucl. Acids Res. 36, W503-508 (2008)
Holt et al; Trends Biotechnol. 2003 November; 21(11):484-90
Huston et al., PNAS USA 85, 5879-5883 (1988)
Korshunov et al, Clinical Science, 2012
Leconet et al., Oncogene, 1-10 (2013)
Lefranc M P. et al., Nucleic Acids Research, 27, 209-212, 1999
Li et al, Oncogene, 28, 3442-3455 (2009)
Iida et al, Anticancer Research, 34:1821-1828 (2014)
Linger et al, Expert Opin. Ther. Targets, 14(10):1073-1090 (2010)
Meyer et al., Sci Signal. 2013 Aug. 6; 6(287):ra66.
Meyers, E. and Miller, W., (1988) Comput. Appl. Biosci 4, 11-17
Needleman and Wunsch, J. Mol. Biol. 48, 444 453 (1970
Paccez et al, Int. J. Cancer: 134, 1024-1033 (2013)
Revets et al; Expert Opin Biol Ther. 2005 January; 5(1):111-24
Ward et al., Nature 341, 544-546 (1989)
Ye et al., Oncogene, 1-11 (2010) (a)
Ye et al., Oncogene (2010) 29, 5254-5264 (b)
Sunshine, J. and Taube, J., Curr. Opin. Pharmacol. (2015) 23, 32-38
O'Donnell et al., Genome Medicine (2016) 8:111
Fundamental Immunology, Ch. 7 (Paul, W., ed., 2nd ed. Raven Press, N.Y. (1989).
Rustin G J, Bast R C, Jr., Kelloff G J, et al., Clinical cancer research: an official journal of the American
Association for Cancer Research. Jun. 1, 2004; 10(11):3919-3926.
Rustin G J, Vergote I, Eisenhauer E, et al., International journal of gynecological cancer: official journal of the International Gynecological Cancer Society. February 2011; 21(2):419-423.
Eisenhauer E A, Therasse P, Bogaerts J, et al., Eur J Cancer. January 2009; 45(2):228-247.
Scher H I, Morris M J, Stadler W M, et al., J Clin Oncol. Apr. 20, 2016; 34(12):1402-1418.
Barbas, C F. J Mol Biol. 1993 Apr. 5; 230(3):812-23
Vink et al., Methods, 65 (1), 5-10 2014
Jorritsma et al. (2007) Blood; 110, 3564-3572
Doronina, S. O. et al. (2003) Nat. Biotechnol. 21, 778-784
WO 2009/063965, Chugai Pharmaceuticals
WO 2010/131733
WO 2011/159980, Genentech
WO 2012/175691, INSERM
WO 2012/175692, INSERM
WO 2013/064685, Pierre Fabré Medicaments
WO 2013/090776, INSERM
WO 2014/174111, Pierre Fabré Medicament and Spirogen Sarl
WO 2016/005593, Genmab

1. An antibody binding to human AXL or an antibody-drug conjugate (ADC) comprising said antibody, for use in treating cancer in a subject, wherein
said cancer is resistant to or is predicted to be or become resistant to;
said cancer has failed to respond to, or is predicted to fail to respond to; and/or
said subject has relapsed after or is predicted to relapse after treatment with an inhibitor of the interaction between a programmed cell death-1 (PD-1) receptor and its ligand.

2. The antibody or ADC for use according to claim 1, wherein said ligand is programmed cell death-ligand 1 (PD-L1) or programmed cell death-ligand 2 (PD-L2).

3. The antibody or ADC for use according to claim 1 or 2, wherein said inhibitor is selected from the group consisting of an antibody, such as a monoclonal antibody, that binds PD-1, an antibody, such as a monoclonal antibody, that binds PD-L1 and an antibody, such as a monoclonal antibody, that binds PD-L2.

4. The antibody or ADC for use according to claim 1, wherein said cancer is a solid tumor, such as a metastasic, solid tumor, such as a metastasic, locally advanced tumor.

5. The antibody or ADC for use according to claim 1 or 2, wherein the cancer is a tumor selected from the group consisting of a melanoma, a carcinoma, a sarcoma (such as an undifferentiated pleomorphic sarcoma, aliposarcoma, a leiomyosarcoma, a synovial sarcoma, a Ewing's sarcoma, an osteosarcoma or a chondrosarcoma), an adenoma, a glioma, a hematologic tumor and a tumor of the lymphoid tissue.

6. The antibody or ADC for use according to claim 1 or 2, wherein the solid tumor is selected from the group consisting of a melanoma, a carcinoma (such as squamous cell carcinoma of the head and neck (SCCHN)), a sarcoma (such as an undifferentiated pleomorphic sarcoma, aliposarcoma, a leiomyosarcoma, a synovial sarcoma, a Ewing's sarcoma, an osteosarcoma or a chondrosarcoma), an adenoma, and a glioma.

7. The antibody or ADC for use according to claim 1 or 2, wherein the solid tumor is selected from the group consisting of a carcinoma, a sarcoma (such as an undifferentiated pleomorphic sarcoma, aliposarcoma, a leiomyosarcoma, a synovial sarcoma, a Ewing's sarcoma, an osteosarcoma, a gastrointestinal stromal tumor (GIST), a rhabdomyosarcoma or a chondrosarcoma), an adenoma, and a glioma.

8. The antibody or ADC for use according to claim 1 or 2, wherein the cancer is selected from the group consisting of endometrial/cervical cancer, lung cancer (such as small cell lung cancer or non-small cell lung cancer), thyroid cancer, colon cancer, kidney cancer, renal cancer, ovary cancer, breast cancer (such as such as estrogen receptor alpha negative cancer, estrogen receptor alpha positive cancer or triple negative breast cancer; i.e. breast cancer tested negative for estrogen receptors (ER−), progesterone receptors (PR−), and human epidermal growth factor receptor 2 (HER2-)), esophagus cancer, skin cancer, melanoma (such as malignant melanoma), pancreatic cancer (such as unresectable advanced or metastatic pancreatic cancer), gastrointestinal stromal tumors (GISTs), and hematological cancer (such as leukemia; e.g. acute lymphoblastic leukemia, acute myeloid leukemia, chronic lymphocytic leukemia or chronic myeloid leukemia).

9. The antibody or ADC for use according to claim 1, wherein said cancer is a metastasic, solid tumor other than melanoma.

10. The antibody or ADC for use according to any of the preceding claims, wherein said subject has documented progressive disease during or after last prior treatment with an inhibitor of the interaction between a programmed cell death-1 (PD-1) receptor and its ligand.

11. The antibody or ADC for use according to any of the preceding claims, wherein the resistance to, the failure to respond to or the relapse from said treatment with an inhibitor of the interaction between a programmed cell death-1 (PD-1) receptor and its ligand is associated with increased expression of AXL.

12. The antibody or ADC for use according to any of the preceding claims, wherein the inhibitor of the interaction between a programmed cell death-1 (PD-1) receptor and its ligand is selected from the group consisting of Opdivo/Nivolumab (Bristol-Myers Squibb), Keytruda/pembrolizumab (Merck & Co), Amp-514/MEDI0680 (Amplimmune), BGB-A317 (BeiGene), REGN2810 (Regeneron), TSR-042 (Tesaro/AnaptysBio), CBT-501/genolimzumab (Genor Bio/CBT Pharma), PF-06801591 (Pfizer), JS-001 (Shanghai Junshi Bio), SHR-1210/INCSHR-1210 (Incyte corp), PDR001 (Novartis), BCD-100 (BioCad), AGEN2034 (Agenus), IBI-308 Innovent Biologics), BI-754091 (Boehringer Ingelheim).

13. The antibody or ADC for use according to any of the preceding claims, wherein the inhibitor of the interaction between a programmed cell death-1 (PD-1) receptor and its ligand is selected from the group consisting of Tecentriq/RG7446; MPDL-3280A, atezolizumab (Roche), Imfinzi/MEDI-4736/durvalumab (AstraZeneca), Bavencio/MSB-0010718C/avelumab (Merck Serono/Pfizer), KN-035-(3DMed/Alphamab Co), CX-072 (CytomX), LY-3300054 (Eli Lilly), MSB0011359C*/M-7824 (Merck KGaA), FAZ053 (Novartis), SHR-1316 (Atridia), ansd CA-170 (Aurigene/Curis).

14. The antibody or ADC for use according to any of the preceding claims, wherein said antibody binding to human AXL or said ADC is provided to the subject as monotherapy.

15. The antibody or ADC for use according to any of the preceding claims, wherein said antibody binding to human AXL or said ADC is provided to the subject as part of a combination therapy.

16. The ADC for use according to any one of the preceding claims, wherein the ADC comprises therapeutic moiety, which is a cytotoxic agent, a chemotherapeutic drug or a radioisotope linked to the antibody optionally with a linker.

17. The ADC for use according to any one of the preceding claims, wherein the therapeutic moiety is a cytotoxic agent, optionally linked to the antibody with a linker.

18. The ADC for use according to claim 17, wherein the cytotoxic agent is linked to the antibody binding to human AXL with a cleavable linker, such as N-succinimydyl 4-(2-pyridyldithio)-pentanoate (SSP), maleimidocaproyl-valine-citrulline-p-aminobenzyloxycarbonyl (mc-vc-PAB) or AV-1 K-lock valine-citrulline.

19. The ADC for use according to any one of claims 17 to 18, wherein the cytotoxic agent is linked to the antibody binding to human AXL with a non-cleavable linker, such as succinimidyl-4(N-maleimidomethyl)cyclohexane-1-carboxylate (MCC) or maleimidocaproyl (MC).

20. The ADC for use according to any one of claims 17 to 19, wherein the cytotoxic agent is selected from the group consisting of DNA-targeting agents, e.g. DNA alkylators and cross-linkers, such as calicheamicin, duocarmycin, rachelmycin (CC-1065), pyrrolo[2,1-c][1,4] benzodiazepines (PBDs), and indolinobenzodiazepine (IGN); microtubule-targeting agents, such as duostatin, such as duostatin-3, auristatin, such as monomethylauristatin E (MMAE) and monomethylauristatin F (MMAF), dolastatin, maytansine, N(2′)-deacetyl-N(2′)-(3-marcapto-1-oxopropyl)-maytansine (DM1), and tubulysin; and nucleoside analogs; or an analogs, derivatives, or prodrugs thereof.

21. The ADC for use according to any one of claims 17 to 20, wherein
(a) the linker is cleavable and the cytotoxic agent has bystander kill capacity;
(b) the linker is cleavable and the cytotoxic agent does not have bystander kill capacity;
(c) the linker is non-cleavable and the cytotoxic agent has bystander kill capacity; or
(d) the linker is non-cleavable and the cytotoxic agent does not have bystander kill capacity.

22. The ADC for use according to any one of claims 16 to 21, wherein the linker is mc-vc-PAB and the cytotoxic agent is MMAE.

23. The ADC for use according to any one of claims 16 to 22, wherein the linker is SSP and the cytotoxic agent is DM1.

24. The ADC for use according to any one of claims 17 to 21, wherein the cytotoxic agent is duostatin-3.

25. The antibody or ADC for use according to any one of the preceding claims, wherein the antibody binding to human AXL does not compete with Growth Arrest-Specific 6 (Gas6) for binding to human AXL.

26. The antibody or ADC for use according to any one of the preceding claims, wherein maximal antibody binding to human AXL in the presence of Gas6 is at least 90%, such as at least 95%, such as at least 97%, such as at least 99%, such as 100%, of binding in the absence of Gas6 as determined by a competition assay, wherein competition between said antibody binding to human AXL and said Gas6 is determined on A431 cells pre-incubated with Gas6 and without Gas6.

27. The antibody or ADC for use according to any one of the preceding claims, wherein the antibody binding to human AXL has a binding affinity (K_{D}) in the range of 0.3×10⁻⁹ to 63×10⁻⁹ M to human AXL, optionally wherein the binding affinity is measured using a Bio-layer Interferometry using soluble AXL extracellular domain.

28. The antibody or ADC for use according to any one of the preceding claims, wherein the antibody binding to human AXL has a dissociation rate of 9.7×10⁻⁵ to 4.4×10⁻³ s⁻¹ to AXL, optionally wherein the dissociation rate is measured by Bio-layer Interferometry using soluble recombinant AXL extracellular domain.

29. The antibody or ADC for use according to any one of the preceding claims, wherein the amino acid sequence of the human AXL is as specified in SEQ ID NO:130.

30. The antibody or ADC for use according to any one of the preceding claims, which binds to cynomolgus monkey AXL as specified in SEQ ID NO:147.

31. The antibody or ADC for use according to any one of the preceding claims, wherein the antibody binding to human AXL comprises at least one binding region comprising a VH region and a VL region selected from the group consisting of:
(a) a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107];
(b) a VH region comprising the CDR1, CDR2, and CDR3 sequences of SEQ ID Nos.: 46, 47, and 48, respectively; and a VL region comprising the CDR1, CDR2, and CDR3 sequences of SEQ ID Nos.: 49, AAS, and 50, respectively, [148];
(c) a VH region comprising the CDR1, CDR2, and CDR3 sequences of SEQ ID Nos.: 114, 115, and 116, respectively, and a VL region comprising the CDR1, CDR2, and CDR3 sequences of SEQ ID Nos.: 117, DAS, and 118, respectively [733];
(d) a VH region comprising the CDR1, CDR2, and CDR3 sequences of SEQ ID Nos.: 51, 52, and 53, respectively; and a VL region comprising the CDR1, CDR2, and CDR3 sequences of SEQ ID Nos.: 55, GAS, and 56, respectively [154];
(e) a VH region comprising the CDR1, CDR2, and CDR3 sequences of SEQ ID Nos.: 51, 52, and 54, respectively; and a VL region comprising the CDR1, CDR2, and CDR3 sequences of SEQ ID Nos.: 55, GAS, and 56, respectively [154-M103L];
(f) a VH region comprising the CDR1, CDR2, and CDR3 sequences of SEQ ID Nos.: 57, 58, and 59, respectively; and a VL region comprising the CDR1, CDR2, and CDR3 sequences of SEQ ID Nos.: 60, GAS, and 61, respectively, [171];
(g) a VH region comprising the CDR1, CDR2, and CDR3 sequences of SEQ ID Nos.: 62, 63, and 64, respectively; and a VL region comprising the CDR1, CDR2, and CDR3 sequences of SEQ ID Nos.: 65, GAS, and 66, respectively, [172];
(h) a VH region comprising the CDR1, CDR2, and CDR3 sequences of SEQ ID Nos.: 67, 68, and 69, respectively; and a VL region comprising the CDR1, CDR2, and CDR3 sequences of SEQ ID Nos.: 70, GAS, and 71, respectively, [181];
(i) a VH region comprising the CDR1, CDR2, and CDR3 sequences of SEQ ID Nos.: 72, 73, and 75, respectively; and a VL region comprising the CDR1, CDR2, and CDR3 sequences of SEQ ID Nos.: 76, ATS, and 77, respectively, [183];
(j) a VH region comprising the CDR1, CDR2, and CDR3 sequences of SEQ ID Nos.: 72, 74, and 75, respectively; and a VL region comprising the CDR1, CDR2, and CDR3 sequences of SEQ ID Nos.: 76, ATS, and 77, respectively, [183-N52Q];
(k) a VH region comprising the CDR1, CDR2, and CDR3 sequences of SEQ ID Nos.: 78, 79, and 80, respectively; and a VL region comprising the CDR1, CDR2, and CDR3 sequences of SEQ ID Nos.: 81, AAS, and 82, respectively, [187];
(l) a VH region comprising the CDR1, CDR2, and CDR3 sequences of SEQ ID Nos.: 83, 84, and 85, respectively; and a VL region comprising the CDR1, CDR2, and CDR3 sequences of SEQ ID Nos.: 86, GAS, and 87, respectively, [608-01];
(m) a VH region comprising the CDR1, CDR2, and CDR3 sequences of SEQ ID Nos.: 88, 89, and 90, respectively; and a VL region comprising the CDR1, CDR2, and CDR3 sequences of SEQ ID Nos.: 91, GAS, and 92, respectively, [610-01];
(n) a VH region comprising the CDR1, CDR2, and CDR3 sequences of SEQ ID Nos.: 93, 94, and 95, respectively; and a VL region comprising the CDR1, CDR2, and CDR3 sequences of SEQ ID Nos.: 96, GAS, and 97, respectively, [613];
(o) a VH region comprising the CDR1, CDR2, and CDR3 sequences of SEQ ID Nos.: 98, 99, and 100, respectively; and a VL region comprising the CDR1, CDR2, and CDR3 sequences of SEQ ID Nos.: 101, DAS, and 102, respectively, [613-08];
(p) a VH region comprising the CDR1, CDR2, and CDR3 sequences of SEQ ID Nos.: 103, 104, and 105, respectively; and a VL region comprising the CDR1, CDR2, and CDR3 sequences of SEQ ID Nos.: 106, GAS, and 107, respectively, [620-06];
(q) a VH region comprising the CDR1, CDR2, and CDR3 sequences of SEQ ID Nos.: 108, 109, and 110, respectively; and a VL region comprising the CDR1, CDR2, and CDR3 sequences of SEQ ID Nos.: 112, AAS, and 113, respectively, [726];
(r) a VH region comprising the CDR1, CDR2, and CDR3 sequences of SEQ ID Nos.: 108, 109, and 111, respectively; and a VL region comprising the CDR1, CDR2, and CDR3 sequences of SEQ ID Nos.: 112, AAS, and 113, respectively, [726-M101L];
(s) a VH region comprising the CDR1, CDR2, and CDR3 sequences of SEQ ID Nos.: 41, 42, and 43, respectively; and a VL region comprising the CDR1, CDR2, and CDR3 sequences of SEQ ID Nos.: 44, AAS, and 45, respectively, [140];
(t) a VH region comprising the CDR1, CDR2, and CDR3 sequences of SEQ ID Nos.: 93, 94, and 95, respectively, and a VL region comprising the CDR1, CDR2, and CDR3 sequences of SEQ ID Nos.: 128, XAS, wherein X is D or G, and 129, respectively, [613/613-08];
(u) a VH region comprising the CDR1, CDR2, and CDR3 sequences of SEQ ID Nos.: 46, 119, and 120, respectively; and a VL region comprising CDR1, CDR2, and CDR3 sequences of SEQ ID Nos.: 49, AAS, and 50, respectively, [148/140];
(v) a VH region comprising the CDR1, CDR2, and CDR3 sequences of SEQ ID Nos.: 123, 124, and 125, respectively; and a VL region comprising CDR1, CDR2, and CDR3 sequences of SEQ ID Nos.: 60, GAS, and 61, respectively [171/172/181]; and
(w) a VH region comprising the CDR1, CDR2, and CDR3 sequences of SEQ ID Nos.: 121, 109, and 122, respectively; and a VL region comprising the CDR1, CDR2, and CDR3 sequences of SEQ ID Nos.: 112, AAS, and 113, respectively [726/187]; and
(x) a VH region comprising the CDR1, CDR2, and CDR3 sequences of SEQ ID Nos.:93, 126, and 127, respectively; and a VL region comprising the CDR1, CDR2, and CDR3 sequences of SEQ ID Nos.: 96, GAS, and 97, respectively [613/608-01/610-01/620-06].

32. The ADC for the use of any one of the preceding claims, wherein the antibody binding to human AXL comprises at least one binding region comprising
(a) a VH region comprising the CDR1, CDR2, and CDR3 sequences of SEQ ID Nos.: 36, 37, and 38, respectively, and
(b) a VL region comprising the CDR1, CDR2, and CDR3 sequences of SEQ ID Nos.: 39, GAS, and 40, respectively [107].

33. The ADC for the use of any one of the preceding claims, wherein the antibody binding to human AXL comprises at least one binding region comprising a VH region and a VL region selected from the group consisting of:
(a) a VH region at least 90%, such as at least 95%, such as at least 97%, such as at least 99% identical to SEQ ID No: 1 and a VL region at least 90%, such as at least 95%, such as at least 97%, such as at least 99% identical to SEQ ID No: 2 [107];
(b) a VH region at least 90%, such as at least 95%, such as at least 97%, such as at least 99% identical to SEQ ID No: 5 and a VL region at least 90%, such as at least 95%, such as at least 97%, such as at least 99% identical to SEQ ID No: 6 [148];
(c) a VH region at least 90%, such as at least 95%, such as at least 97%, such as at least 99% identical to SEQ ID No: 34 and a VL region at least 90%, such as at least 95%, such as at least 97%, such as at least 99% identical to SEQ ID No: 35 [733]
(d) a VH region at least 90%, such as at least 95%, such as at least 97%, such as at least 99% identical to SEQ ID No: 7 and a VL region at least 90%, such as at least 95%, such as at least 97%, such as at least 99% identical to SEQ ID No: 9 [154];
(e) a VH region at least 90%, such as at least 95%, such as at least 97%, such as at least 99% identical to SEQ ID No: 10 and a VL region at least 90%, such as at least 95%, such as at least 97%, such as at least 99% identical to SEQ ID No: 11 [171];
(f) a VH region at least 90%, such as at least 95%, such as at least 97%, such as at least 99% identical to SEQ ID No: 16 and a VL region at least 90%, such as at least 95%, such as at least 97%, such as at least 99% identical to SEQ ID No: 18 [183];
(g) a VH region at least 90%, such as at least 95%, such as at least 97%, such as at least 99% identical to SEQ ID No: 25 and a VL region at least 90%, such as at least 95%, such as at least 97%, such as at least 99% identical to SEQ ID No: 26 [613];
(h) a VH region at least 90%, such as at least 95%, such as at least 97%, such as at least 99% identical to SEQ ID No: 31 and a VL region at least 90%, such as at least 95%, such as at least 97%, such as at least 99% identical to SEQ ID No: 33 [726];
(i) a VH region at least 90%, such as at least 95%, such as at least 97%, such as at least 99% identical to SEQ ID No: 3 and a VL region at least 90%, such as at least 95%, such as at least 97%, such as at least 99% identical to SEQ ID No: 4 [140];
(j) a VH region at least 90%, such as at least 95%, such as at least 97%, such as at least 99% identical to SEQ ID No:8 and a VL region at least 90%, such as at least 95%, such as at least 97%, such as at least 99% identical to SEQ ID No:9 [154-M103L];
(k) a VH region at least 90%, such as at least 95%, such as at least 97%, such as at least 99% identical to SEQ ID No:12 and a VL region at least 90%, such as at least 95%, such as at least 97%, such as at least 99% identical to SEQ ID No:13 [172];
(l) a VH region at least 90%, such as at least 95%, such as at least 97%, such as at least 99% identical to SEQ ID No:14 and a VL region at least 90%, such as at least 95%, such as at least 97%, such as at least 99% identical to SEQ ID No:15 [181];
(m) a VH region at least 90%, such as at least 95%, such as at least 97%, such as at least 99% identical to SEQ ID No:17 and a VL region at least 90%, such as at least 95%, such as at least 97%, such as at least 99% identical to SEQ ID No:18 [183-N52Q];
(n) a VH region at least 90%, such as at least 95%, such as at least 97%, such as at least 99% identical to SEQ ID No:19 and a VL region at least 90%, such as at least 95%, such as at least 97%, such as at least 99% identical to SEQ ID No:20 [187];
(o) a VH region at least 90%, such as at least 95%, such as at least 97%, such as at least 99% identical to SEQ ID No:21 and a VL region at least 90%, such as at least 95%, such as at least 97%, such as at least 99% identical to SEQ ID No:22 [608-01];
(p) a VH region at least 90%, such as at least 95%, such as at least 97%, such as at least 99% identical to SEQ ID No:23 and a VL region at least 90%, such as at least 95%, such as at least 97%, such as at least 99% identical to SEQ ID No:24 [610-01];
(q) a VH region at least 90%, such as at least 95%, such as at least 97%, such as at least 99% identical to SEQ ID No:27 and a VL region at least 90%, such as at least 95%, such as at least 97%, such as at least 99% identical to SEQ ID No:28 [613-08];
(r) a VH region at least 90%, such as at least 95%, such as at least 97%, such as at least 99% identical to SEQ ID No:29 and a VL region at least 90%, such as at least 95%, such as at least 97%, such as at least 99% identical to SEQ ID No:30 [620-06]; and
(s) a VH region at least 90%, such as at least 95%, such as at least 97%, such as at least 99% identical to SEQ ID No:32 and a VL region at least 90%, such as at least 95%, such as at least 97%, such as at least 99% identical to SEQ ID No:33 [726-M101L].

34. The antibody or ADC for use according to any one of the preceding claims, wherein the at least one binding region of the antibody comprises a VH region and a VL region selected from the group consisting of;
(a) a VH region comprising SEQ ID No: 1 and a VL region comprising SEQ ID No: 2 [107];
(b) a VH region comprising SEQ ID No: 5 and a VL region comprising SEQ ID No: 6 [148];
(c) a VH region comprising SEQ ID No: 34 and a VL region comprising SEQ ID No: 35 [733]
(d) a VH region comprising SEQ ID No: 7 and a VL region comprising SEQ ID No: 9 [154];
(e) a VH region comprising SEQ ID No: 10 and a VL region comprising SEQ ID No: 11 [171];
(f) a VH region comprising SEQ ID No: 16 and a VL region comprising SEQ ID No: 18 [183];
(g) a VH region comprising SEQ ID No: 25 and a VL region comprising SEQ ID No: 26 [613];
(h) a VH region comprising SEQ ID No: 31 and a VL region comprising SEQ ID No: 33 [726];
(i) a VH region comprising SEQ ID No: 3 and a VL region comprising SEQ ID No: 4 [140];
(j) a VH region comprising SEQ ID No:8 and a VL region comprising SEQ ID No:9 [154-M103L];
(k) a VH region comprising SEQ ID No:12 and a VL region comprising SEQ ID No:13 [172];
(l) a VH region comprising SEQ ID No:14 and a VL region comprising SEQ ID No:15 [181];
(m) a VH region comprising SEQ ID No:17 and a VL region comprising SEQ ID No:18 [183-N52Q];
(n) a VH region comprising SEQ ID No:19 and a VL region comprising SEQ ID No:20 [187];
(o) a VH region comprising SEQ ID No:21 and a VL region comprising SEQ ID No:22 [608-01];
(p) a VH region comprising SEQ ID No:23 and a VL region comprising SEQ ID No:24 [610-01];
(q) a VH region comprising SEQ ID No:27 and a VL region comprising SEQ ID No:28 [613-08];
(r) a VH region comprising SEQ ID No:29 and a VL region comprising SEQ ID No:30 [620-06]; and
(s) a VH region comprising SEQ ID No:32 and a VL region comprising SEQ ID No:33 [726-M101L].

35. The antibody or ADC for use according to any one of the preceding claims, wherein the at least one binding region of the antibody binding to human AXL comprises a VH region comprising SEQ ID No: 1 and a VL region comprising SEQ ID No: 2 [107].

36. The antibody or ADC for use according to any one of the preceding claims, wherein the antibody binding to human AXL comprises at least one binding region comprising a VH region comprising the CDR1, CDR2, and CDR3 sequences of SEQ ID Nos.: 36, 37, and 38, respectively; and a VL region comprising the CDR1, CDR2, and CDR3 sequences of SEQ ID Nos.: 39, GAS, and 40, respectively, [107].

37. The antibody or ADC for use according to any one of the preceding claims, wherein the antibody binds to an epitope on AXL wherein the epitope is recognized by any of the antibodies defined in any one of claims 31 to 36.

38. The antibody or ADC for use according to any one of the preceding claims, wherein the antibody binding to human AXL binds to an epitope within the Ig1 domain, or Ig1-like domain, of AXL, the epitope comprising or requiring one or more amino acids corresponding to positions L121 to Q129 or T112 to Q124 of human AXL.

39. The antibody or ADC for use according to any one of claims 1 to 37, wherein the antibody binding to human AXL binds to an epitope within the Ig2 domain or Ig2-like domain, of AXL, the epitope comprising or requiring the amino acids corresponding to position D170 or the combination of D179 and one or more amino acids corresponding to positions T182 to R190 of human AXL.

40. The ADC for use according to any one of claims 1 to 37, wherein the antibody binding to human AXL binds to an epitope within the FN1 domain, or FN-like domain, of human AXL, the epitope comprises or requires one or more amino acids corresponding to positions Q272 to A287 and G297 to P301 of human AXL.

41. The antibody or ADC for the use of any one of claims 1 to 37, wherein the antibody binding to human AXL binds to an epitope within the FN2 domain of human AXL, the epitope comprises or requires the amino acids corresponding to positions A359, R386, and one or more amino acids corresponding to positions Q436 to K439 of human AXL.

42. The antibody or ADC for the use according to any one of the preceding claims, wherein the ACD is able to induce tumor regression in an SKMel-147 human xenograft mouse model and/or in a BLM melanoma xenograft model.

43. The antibody or ADC for the use according to claim 42, wherein the SKMel-147 human xenograft mouse model and/or the BLM melanoma xenograft model is/are resistant to anti-PD-1 treatment, such as treatment with an inhibitor of the interaction between a programmed cell death-1 (PD-1) receptor and its ligand.

44. The antibody or ADC for use according to claim 42 or 43, wherein the SKMel-14 human xenograft mouse model is generated at described in Example 5 herein or essentially as described in Example 5 herein.

45. The antibody or ADC for use according to claim 42 or 43, wherein the BLM melanoma xenograft model is generated as described in Example 6 herein or essentially as described in Example 6 herein.

46. The antibody or ADC for the use of any of the preceding claims, wherein the antibody binding to human AXL comprises a heavy chain of an isotype selected from the group consisting of IgG1, IgG2, IgG3, and IgG4.

47. The antibody or ADC for use according to claim 46, wherein the isotype of the antibody binding to human AXL is IgG1, such as human IgG1, optionally allotype IgG1m(f).

48. The antibody or ADC for use according to any one of the preceding claims, wherein the antibody binding to human AXL is a monoclonal antibody or an antigen-binding fragment thereof, such as a full-length monoclonal antibody, such as a full-length monoclonal IgG1,κ antibody.

49. The antibody or ADC for use according to any one of the preceding claims, wherein the antibody is a humanized or human antibody.

50. The antibody or ADC for use according to any one of the preceding claims, wherein the antibody is Enapotamab.

51. The antibody or ADC for use according to any one of the preceding claims, wherein the ADC is Enapotamab vedotin.

52. The antibody or ADC for use according to any one of claims 1 to 43, wherein the antibody binding to human AXL is an effector-function-deficient antibody, a stabilized IgG4 antibody or a monovalent antibody.

53. The antibody or ADC for the use according to any one of the preceding claims, wherein the heavy chain of the antibody binding to human AXL has been modified such that the entire hinge region has been deleted.

54. The antibody or ADC for use according to any one of the preceding claims, wherein the sequence of the antibody binding to human AXL has been modified so that it does not comprise any acceptor sites for N-linked glycosylation.

55. The antibody or ADC for use according to any one of the preceding claims, wherein the antibody binding to human AXL is a single-chain antibody.

56. The antibody or ADC for use according to any one of the preceding claims, wherein the antibody binding to human AXL is a bispecific antibody comprising a first binding region of an antibody according to any one of the preceding claims, and a second binding region which binds a different target or epitope than the first binding region.

57. The antibody or ADC for use according to claim 49, wherein the bispecific antibody binding to human AXL comprises a first and a second heavy chain, each of the first and second heavy chain comprises at least a hinge region, a CH2 and CH3 region, wherein in the first heavy chain at least one of the amino acids in the positions corresponding to positions selected from the group consisting of K409, T366, L368, K370, D399, F405, and Y407 in a human IgG1 heavy chain has been substituted, and in the second heavy chain at least one of the amino acids in the positions corresponding to a position selected from the group consisting of F405, T366, L368, K370, D399, Y407, and K409 in a human IgG1 heavy chain has been substituted, and wherein the substitutions of the first and the second heavy chains are not in the same positions.

58. The antibody or ADC for use according to any one of the preceding claims, wherein the amino acid in the position corresponding to K409 in a human IgG1 heavy chain is R in the first heavy chain, and the amino acid in the position corresponding to F405 in a human IgG1 heavy chain is L in the second heavy chain, or vise versa.

59. The antibody or ADC for use according to any one of the preceding claims, wherein the antibody or ADC is in a formulation, such as a formulation comprising one or more pharmaceutically acceptable excipients, such as a pharmaceutical formulation,

60. The antibody or ADC for use according to any one of the preceding claims, wherein the antibody or ADC is in a lyophilized formulation.

61. The antibody or ADC for use according to claim 53, wherein the lyophilized formulation is obtainable or obtained by lyophilizing an aqueous formulation comprising the antibody or ADC and one or more excipients, wherein the aqueous formulation is free of any surfactant.

62. The antibody or ADC for use according to any one of claims 53 to 54, wherein the lyophilized formulation is obtainable or obtained by lyophilizing an aqueous formulation comprising the antibody or ADC and
a. a buffer providing for a pH of between about 5 and about 7 in the aqueous formulation;
b. at least one bulking agent; and
c. at least one non-reducing sugar which forms an amorphous phase with the antibody or ADC in solid state.

63. The antibody or ADC for use according to any one of claims 54 to 55, wherein the aqueous formulation is free of any surfactant.

64. The antibody or ADC for use according to any one of claims 54 to 56, wherein the aqueous formulation comprises a buffer selected from the group consisting of histidine, citrate, 2-(N-morpholino)ethanesulfonic acid (MES), succinate, glycolate, carbonic acid and phosphate, or a combination of any thereof, wherein the pH of the aqueous formulation is in a range from about 5 to about 7.

65. The antibody or ADC for use according to any one of claims 54 to 57, wherein the aqueous formulation comprises a histidine buffer.

66. The antibody or ADC for use according to any one of claims 54 to 58, wherein the aqueous formulation comprises a buffer at a concentration of about 5 mM to about 100 mM, such as from about 10 mM to about 50 mM buffer, such as from about 20 mM to about 40 mM, such as from about 28 mM to about 32 mM, such as about 30 mM buffer.

67. The antibody or ADC for use according to any one of claims 53 to 59, wherein the lyophilized formulation comprises a bulking agent selected from mannitol, glycine, and a combination thereof.

68. The antibody or ADC for use according to any one of claims 53 to 60, wherein the lyophilized formulation, comprises mannitol.

69. The antibody or ADC for use according to any one of claims 54 to 61, wherein the aqueous formulation comprises a bulking agent at a concentration of about 1% (w/v) to about 5% (w/v), such as about 2% (w/v) to about 4% (w/v), such as from about 2.5% (w/v) to about 3.5% (w/v), such as about 3% (w/v).

70. The antibody or ADC for use according to any one of claims 53 to 62, wherein the aqueous formulation comprises a bulking agent at a concentration of about 50 mM to about 300 mM, such as from about 100 mM to about 225 mM, such as from about 150 mM to about 180 mM, such as about 165 mM.

71. The antibody or ADC for use according to any one of claims 53 to 63, wherein the lyophilized formulation of any one of the preceding claims, comprising a non-reducing sugar selected from sucrose, trehalose, and a combination thereof.

72. The antibody or ADC for use according to any one of claims 53 to 64, wherein the lyophilized formulation comprises sucrose.

73. The antibody or ADC for use according to any one of claims 54 to 65, wherein the aqueous formulation comprises a non-reducing sugar at a concentration of about 0.5% (w/v) to about 7% (w/v), such as from about 0.5% (w/v) to about 4% (w/v), such as from about 1% (w/v) to about 3% (w/v) or from about 2.5% to about 3.5%, such as about 3% (w/v).

74. The antibody or ADC for use according to any one of claims 54 to 66, wherein the aqueous formulation comprises a non-reducing sugar at a concentration of about 15 mM to about 200 mM, such as from about 30 mM to about 150 mM, such as 80 mM to about 100 mM, such as from about 70 to about 90 mM, such as from about 84 mM to about 92 mM sucrose, such as about 88 mM.

75. The antibody or ADC for use according to any one of claims 53 to 67, wherein the lyophilized formulation is obtainable or obtained by lyophilizing an aqueous formulation, wherein the antibody or ADC concentration in the aqueous formulation is from about 5 mg/mL to about 30 mg/mL, such as from about 7 mg/mL to about 20 mg/mL, such as from about 8 mg/mL to about 15 mg/mL, such as from about 9 mg/mL to about 11 mg/mL, such as about 10 mg/mL.

76. The antibody or ADC for use according to any one of claims 53 to 68, wherein the lyophilized formulation is obtainable or obtained by lyophilizing an aqueous formulation in which the pH is in a range from about 5.5 to 6.5, such as about 6.

77. The antibody or ADC for use according to any one of claims 53 to 69, wherein the lyophilized formulation is obtainable or obtained by lyophilizing an aqueous formulation having a pH of about 5 to about 7 and comprising
a. from about 5 mg/mL to about 30 mg/mL of the antibody or ADC;
b. from about 10 mM to about 50 mM histidine;
c. from about 30 mM to about 150 mM sucrose or trehalose; and
d. from about 150 mM to about 180 mM mannitol or glycine.

78. The antibody or ADC for use according to any one of claims 54 to 70, wherein the aqueous formulation has a pH in the range of about 5.5 to about 6.5 and comprises
a. from about 9 mg/mL to about 11 mg/mL of the antibody or ADC, such as about 10 mg/mL of the antibody or ADC;
b. from about 20 mM to about 40 mM histidine, such as about 30 mM histidine;
c. from about 80 mM to about 100 mM sucrose, such as about 88 mM sucrose; and
d. from about 150 mM to about 180 mM mannitol, such as about 165 mM; and
wherein the aqueous formulation is free of any surfactant.

79. The antibody or ADC for use according to any one of claims 54 to 71, wherein the antibody or ADC in said lyophilized formulation is stable at 2-8° C., such as at 5° C. for pharmaceutical use for at least 6 months, such as for at least 9 months, such as for at least 15 months or preferably for at least 18 months, or even more preferred for at least 24 months, or most preferred for at least 36 months.

80. The antibody or ADC for use according to any one of claims 54 to 72, wherein the lyophilized formulation is stable when it has less than 10% aggregates, such as less than 5.0% aggregates, such as less than 3.0% aggregates, such as less than 2.0% aggregates when stored at 5° C. for at least 6 months, such as for at least 9 months, such as for at least 15 months or preferably for at least 18 months, or even more preferred for at least 24 months, or most preferred for at least 36 months.

81. The antibody or ADC for use according to claim 73, wherein the stability is determined by size-exclusion analysis, cIEF, or both.

82. The antibody or ADC for use according to any one of claims 53 to 74, wherein the lyophilized formulation contains less than 3.0% moisture, such as less than 2.0% moisture, such as less than 1% moisture, or less than 0.5% moisture.

83. The antibody or ADC for use according to any one of claims 53 to 75, wherein the lyophilized formulation is free of any inorganic salts.

84. The antibody or ADC for use according to any one of claims 52 to 76, wherein the pharmaceutical formulation is obtained or obtainable by reconstituting the lyophilized formulation as defined in any one of claims 53 to 75 in a sterile aqueous diluent.

85. The antibody or ADC for use according to any one of claims 52 to 77, wherein the pharmaceutical formulation has a pH of about 5 to about 7 and comprising, in aqueous solution:
a. from about 5 mg/mL to about 30 mg/mL of the antibody or ADC;
b. from about 10 mM to about 50 mM histidine;
c. from about 30 mM to about 150 mM sucrose or trehalose; and
d. from about 50 mM to about 300 mM mannitol or glycine.

86. The antibody or ADC for use according to any one of claims 52 to 77, wherein the pharmaceutical formulation has a pH in the range of about 5.5 to about 6.5 and comprises:
a. from about 9 mg/mL to about 11 mg/mL of the antibody or ADC, such as about 10 mg/mL of the antibody or ADC;
b. from about 20 mM to about 40 mM histidine, such as about 30 mM histidine;
c. from about 80 mm to about 100 mM sucrose, such as about 88 mM sucrose; and
d. from about 150 mM to about 180 mM mannitol, such as about 165 mM;
wherein the aqueous formulation is free of any surfactant.

87. The antibody or ADC for use according to any one of the preceding claims, wherein the antibody or ADC is in an aqueous formulation comprising one or more pharmaceutically acceptable excipients, wherein the aqueous formulation is free of any surfactant.

88. The antibody or ADC for use according to any one of the preceding claims, wherein the antibody or ADC is in an aqueous formulation comprising a buffer and at least one stabilizer, wherein the pH of the aqueous formulation is between about 5 and about 7 and wherein the aqueous formulation is free of any surfactant.

89. The antibody or ADC for use according to any one of the preceding claims, wherein the antibody or ADC is in an aqueous formulation comprising a buffer selected from the group consisting of histidine, citrate, MES, phosphate, carbonic acid, succinate, glycolate, or a combination of any thereof, wherein the pH of the aqueous formulation is in a range from about 5 to about 7.

90. The antibody or ADC for use according to any one of the preceding claims, wherein the antibody or ADC is in an aqueous formulation, comprising a histidine buffer.

91. The antibody or ADC for use according to any one of the preceding claims, wherein the antibody or ADC is in an aqueous formulation comprising a buffer at a concentration of about 10 mM to about 50 mM, such as from about 20 mM to about 40 mM buffer, such as from about 28 mM to about 34 mM, such as from about 29 mM to about 31 mM, such as about 30 mM.

92. The antibody or ADC for use according to any one of the preceding claims, wherein the antibody or ADC is in an aqueous formulation, comprising a stabilizer selected from the group consisting of mannitol, sucrose and trehalose.

93. The antibody or ADC for use according to any one of the preceding claims, wherein the antibody or ADC is in an aqueous formulation, comprising a stabilizer which is mannitol.

94. The antibody or ADC for use according to any one of the preceding claims, wherein the antibody or ADC is in an aqueous formulation, comprising a stabilizer at a concentration of about 20 mM to about 200 mM, such as from about 30 mM to about 100 mM, such as from about 40 mM to about 80 mM, such as about 50 mM to about 60 mM, such as about 55 mM.

95. The antibody or ADC for use according to any one of the preceding claims, wherein the antibody or ADC is in an aqueous formulation comprising a stabilizer selected from sucrose, trehalose and a combination thereof.

96. The antibody or ADC for use according to any one of the preceding claims, wherein the antibody or ADC is in an aqueous formulation, which is free of any one or more of arginine, glycine, glutamic acid, sorbitol, trehalose, sucrose and sodium chloride.

97. The antibody or ADC for use according to any one of the preceding claims, wherein the antibody or ADC is in an aqueous formulation, wherein the antibody or ADC concentration is from about 5 mg/mL to about 40 mg/mL, such as from about 8 mg/mL to about 35 mg/mL, such as from about 10 mg/mL to about 30 mg/mL, such as from about 15 mg/mL to about 25 mg/mL, such as about 20 mg/m L.

98. The antibody or ADC for use according to any one of the preceding claims, wherein the antibody or ADC is in an aqueous formulation, wherein the pH of the aqueous formulation is in a range from about 5.5 to 6.5, such as about 6.

99. The antibody or ADC for use according to any one of the preceding claims, wherein the antibody or ADC is in an aqueous formulation having a pH of about 5 to about 7 and comprising
a. from about 5 mg/mL to about 40 mg/mL of the antibody or ADC and
b. from about 10 mM to about 50 mM histidine;
c. from about 50 mM to about 300 mM mannitol.

100. The antibody or ADC for use according to any one of the preceding claims, wherein the antibody or ADC is in an aqueous formulation, which has a pH in the range of about 5.5 to about 6.5 and comprises
a. from about 15 mg/mL to about 25 mg/mL of the antibody or ADC, such as about 20 mg/mL of the antibody or ADC;
b. from about 20 mM to about 40 mM histidine, such as about 30 mM histidine;
c. from about 50 mM to about 60 mM mannitol, such as about 55 mM,
wherein the aqueous formulation is free of any added surfactant, amino acid excipient, NaCl, or a combination of any thereof.

101. The antibody or ADC for use according to any one of the preceding claims, wherein the antibody or ADC is in a frozen aqueous formulation, which is obtained or obtainable by freezing the aqueous formulation defined in any one of claims XX to XX.

102. The antibody or ADC for use according to any of the preceding claims, wherein the antibody or ADC is administered to said subject in therapeutically effective amounts and frequencies, such as
In at least one cycle comprising administration once every three weeks, such as on day 1 of a cycle of 21 days; or
in at least one cycle comprising administration once a week for three consecutive weeks followed by a one-week resting period without any administration of ADC so that each cycle time is 28 days including the resting period, such as on days 1, 8 and 15 in the cycle of 28 days.

103. The antibody or ADC for use according to claim 95, wherein the dose of the antibody or ADC in said cycle of 21 days is between 0.6 mg/kg and 4.0 mg/kg of the subject's body weight, such as between 0.6 mg/kg and 3.2 mg/kg of the subject's body weight, such as at a dose of about 0.6 mg/kg or at a dose of about 0.8 mg/kg or at a dose of about 1.0 mg/kg or at a dose of about 1.2 mg/kg or at a dose of about 1.4 mg/kg or at a dose of about 1.6 mg/kg or at a dose of about 1.8 mg/kg or at a dose of about 2.0 mg/kg or at a dose of about 2.2 mg/kg or at a dose of about 2.4 mg/kg or at a dose of about 2.6 mg/kg or at a dose of about 2.8 mg/kg or at a dose of about 3.0 mg/kg or at a dose of about 3.2 mg/kg.

104. The antibody or ADC for use according to claim 95, wherein the dose of the antibody or ADC in said cycle of 28 days is between 0.45 mg/kg and 2.0 mg/kg of the subject's body weight, such as at a dose of 0.45 mg/kg or at a dose of 0.5 mg/kg or at a dose of 0.6 mg/kg or at a dose of 0.7 mg/kg or at a dose of 0.8 mg/kg or at a dose of 0.9 mg/kg or at a dose of 1.0 mg/kg or at a dose of 1.1 mg/kg or at a dose of 1.2 mg/kg or at a dose of 1.3 mg/kg or at a dose of 1.4 mg/kg or at a dose of 1.5 mg/kg or at a dose of 1.6 mg/kg or at a dose of 1.7 mg/kg or at a dose of 1.8 mg/kg or at a dose of 1.9 mg/kg or at a dose of 2.0 mg/kg.

105. The antibody or ADC for use according to any one of claims 95 to 97, wherein the number of cycles of 21 days or the number of cycles of 28 days is between 2 and 48, such as between 2 and 36, such as between 2 and 24, such as between 2 and 15, such as between 2 and 12, such as 2 cycles, 3 cycles, 4 cycles, 5 cycles, 6 cycles, 7 cycles, 8 cycles, 9 cycles, 10 cycles, 11 cycles or 12 cycles.

106. The antibody or ADC for use according to any one of claims 1 to 97, wherein the antibody or ADC is administered for at least four treatment cycles of 28 days, wherein the antibody or ADC in each treatment cycle is administered once a week at a dose of 0.45 mg/kg body weight, such as at a dose of 0.6 mg/kg body weight, 0.8 mg/kg body weight, 1.0 mg/kg body weight, 1.2 mg/kg body weight, 1.4 mg/kg body weight, 1.6 mg/kg body weight, 1.8 mg/kg body weight, or such as 2.0 mg/kg body weight for three consecutive weeks followed by a resting week without any administration of the antibody or ADC.

107. The conjugate for use according to any one of the preceding claims, wherein the conjugate is administered to the subject at a dose of about 2.0-about 2.4 mg/kg body weight once every three weeks or by weekly dosing of about 0.6-about 1.4 mg/kg body weight for three weeks, optionally followed by one treatment-free week.

108. The conjugate for use according to any one of the preceding claims, wherein the conjugate is administered to the subject at a dose of about 2.2 mg/kg body weight once every three weeks or by weekly dosing of about 1.0 mg/kg body weight for three weeks, optionally followed by one treatment-free week.

109. The conjugate for use according to any one of the preceding claims, wherein the conjugate is administered to the subject by weekly dosing of about 0.4-1.0 mg/kg body weight.

110. The conjugate for use according to any one of the preceding claims, wherein the conjugate is administered to the subject by weekly dosing of about 0.6-1.0 mg/kg body weight.

111. The conjugate for use according to any one of the preceding claims, wherein the conjugate is administered to the subject by weekly dosing of about 0.4-0.8 mg/kg body weight.

112. The conjugate for use according to any one of the preceding claims, wherein the conjugate is administered to the subject by weekly dosing of about 0.5-0.7 mg/kg body weight.

113. The conjugate for use according to any one of the preceding claims, wherein the conjugate is administered to the subject by weekly dosing of about 0.6 mg/kg body weight.

114. The conjugate for use according to any one of the preceding claims, wherein the route of administration is intravenous.

115. The conjugate for use according to any one of the preceding claims, wherein treatment is continued at least until said subject has experienced progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the first dose of the conjugate.

116. The conjugate for use according to any one of the preceding claims, wherein treatment is continued until disease progression or unacceptable toxicity.

117. An antibody binding to human AXL or an antibody-drug conjugate (ADC) comprising an antibody binding to human AXL, for use in the manufacture of a medicament for treating cancer in a subject, wherein
said cancer is resistant to or is predicted to be or become resistant to;
said cancer has failed to respond to, or is predicted to fail to respond to; and/or
said subject has relapsed after or is predicted to relapse after
treatment with an inhibitor of the interaction between a programmed cell death-1 (PD-1) receptor and its ligand.

118. The antibody or ADC for use in the manufacture of a medicament according to claim 100, wherein
the ligand is as defined in claim 2;
the inhibitor of the interaction between a programmed cell death-1 (PD-1) receptor and its ligand is as defined in any one of claims 3, 12 and 13;
the cancer is as defined in any one of claims 4 to 9;
the subject is as defined in any one of claims 10 to 11;
antibody or ADC is as defined in any one of claims 14-58;
the formulation is as defined in any one of claims 59 to 101; and/or
the amounts and frequencies in which the antibody or ADC is administered to said subject is as defined in any one of claims 102 to 116.

119. A method of treating cancer in a subject, wherein
said cancer is resistant to or is predicted to be or become resistant to;
said cancer has failed to respond to, or is predicted to fail to respond to; and/or
said subject has relapsed after or is predicted to relapse after
treatment with an inhibitor of the interaction between a programmed cell death-1 (PD-1) receptor and its ligand;
the method comprising administering to said subject a therapeutically effective amount of an antibody binding to human AXL or an antibody-drug conjugate (ADC) comprising an antibody binding to human AXL.

120. The method of treating cancer according to claim 102, wherein
the ligand is as defined in claim 2;
the inhibitor of the interaction between a programmed cell death-1 (PD-1) receptor and its ligand is as defined in any one of claims 3, 12 and 13;
the cancer is as defined in any one of claims 4 to 9;
the subject is as defined in any one of claims 10 to 11;
antibody or ADC is as defined in any one of claims 14-58;
the formulation is as defined in any one of claims 59 to 101; and/or
the amounts and frequencies in which the antibody or ADC is administered to said subject is as defined in any one of claims 102 to 116.


SEQUENCE LISTING (147 sequences)

SEQ ID NO: 1 - protein, 116 aa, homo sapiens
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSS

SEQ ID NO: 2 - protein, 108 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 3 - protein, 120 aa, homo sapiens
EVQLLESGGGLVQPGGSLRLSCAASGFTFSSYAMTWVRQAPGKGLEWVSAISISGASTFYADSVKGRFTISRDNSKNTLSLQMNSLRAEDTAVYFCRGYSGYVYDAFDIWGQGTMVTVSS

SEQ ID NO: 4 - protein, 107 aa, homo sapiens
DIQMTQSPSSLSASVGDRVTITCRASQGISNWLAWYQQKPEKAPKSLIYAASSLQSGVPSRFSGSGSGTDFTLTISSLQPEDFATYYCQQYNSYPLTFGGGTKVEIK

SEQ ID NO: 5 - protein, 120 aa, homo sapiens
EVQLLESGGGLVQPGGSLRLSCAASGFTFSSYAMTWVRQAPGKGLEWVSAISISGGSTFYADSVKGRFTISRDNSKNTLYLQMNSLRAEDTAVYYCRGYSGYVYDAFDFWGQGTMVTVSS

SEQ ID NO: 6 - protein, 107 aa, homo sapiens; identical to SEQ ID NO: 4

SEQ ID NO: 7 - protein, 121 aa, homo sapiens
EVQLLDSGGGLVQPGGSLRLSCAASGFTFSSYAMSWVRQAPGKGLEWVSAISIGGGNAYYADSVKGRFTISRDNSKNTLYLQMNSLRAADTAVYYCAKPGFIMVRGPLDYWGQGALVTVSS

SEQ ID NO: 8 - protein, 121 aa, homo sapiens
EVQLLDSGGGLVQPGGSLRLSCAASGFTFSSYAMSWVRQAPGKGLEWVSAISIGGGNAYYADSVKGRFTISRDNSKNTLYLQMNSLRAADTAVYYCAKPGFILVRGPLDYWGQGALVTVSS

SEQ ID NO: 9 - protein, 108 aa, homo sapiens
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPYTFGQGTKLEIK

SEQ ID NO: 10 - protein, 125 aa, homo sapiens
EVQLLESGGGLVQPGGSLRLSCAASGFTFSSYAMSWVRQAPGKGLEWVSDISVSGGSTYYADSVKGRFTISRDNSKNTLYLQMNSLRAEDTAVYYCAKEGYIWFGESLSYAFDIWGQGTMVTVSS

SEQ ID NO: 11 - protein, 107 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGRSFTFGPGTKVDIK

SEQ ID NO: 12 - protein, 125 aa, homo sapiens
EVQLLESGGGLVQPGGSLRLSCAASGFTFSNYAMSWVRQAPGKGLEWVSDISVSGGSTYYADSVKGRFTISRDNSKNTLYLQMNSLRAEDTAVYYCAKEGYIWFGESLSYAFDIWGQGTMVTVSS

SEQ ID NO: 13 - protein, 107 aa, homo sapiens; identical to SEQ ID NO: 11

SEQ ID NO: 14 - protein, 125 aa, homo sapiens
EVQLLESGGGLVQPGGSLRLSCAASGFTFSSYAMSWVRQAPGKGLEWVSDISVSGGSTYYADSVKGRFTISRDNSKNTLYLHMNSLRAEDTAVYYCAKEGYIWFGESLSYAFDIWGQGTMVTVSS

SEQ ID NO: 15 - protein, 107 aa, homo sapiens; identical to SEQ ID NO: 11

SEQ ID NO: 16 - protein, 117 aa, homo sapiens
QVQLQQWGAGLLKPSETLSLTCAVYGGSFSGYYWSWIRQPPGKGLEWIGEINQSGSTNYNPSLKSRVTISVDTSKNQFSLKLSSVTAADTSVYYCASGNWDHFFDYWGQGTLVTVSS

SEQ ID NO: 17 - protein, 117 aa, homo sapiens
QVQLQQWGAGLLKPSETLSLTCAVYGGSFSGYYWSWIRQPPGKGLEWIGEIQQSGSTNYNPSLKSRVTISVDTSKNQFSLKLSSVTAADTSVYYCASGNWDHFFDYWGQGTLVTVSS

SEQ ID NO: 18 - protein, 107 aa, homo sapiens
DIQMTQSPSSVSASVGDRVTITCRASQGISSWLAWYQHKPGKAPKLLIYATSSLQSGVTSRFSGSGSGTDFTLTISSLQPEDFATYYCQQAKSFPWTFGQGTKVEIK

SEQ ID NO: 19 - protein, 123 aa, homo sapiens
QVPLQQWGAGLLKPSETLSLTCAVYGGSFSGYHWSWIRQPPGKGLEWIGEISHSGRTNYNPSLKSRVTISIDTSKNQFSLKLSSVTAADTAVYYCASFITMIRGTIITHFDYWGQGTLVTVSS

SEQ ID NO: 20 - protein, 107 aa, homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYHSYPYTFGQGTKLEIK

SEQ ID NO: 21 - protein, 124 aa, homo sapiens
QVQLVQSGAEVKKPGSSVKVSCKASGGTFSSYAISWVRQAPGQGLEWMGRIIPIFGIANYVQKFQGRVTITADKSTSTAYMELSSLRAEDTAVYYCARRGDYYGSGSPDVFDIWGQGTMVTVSS

SEQ ID NO: 22 - protein, 107 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSYTFGQGTKLEIK

SEQ ID NO: 23 - protein, 124 aa, homo sapiens
QVQLVQSGAEVKKPGSSVKVSCKASGGTFSSYAISWVRQAPGQGLEWMGRIIPIFGIANYVQKFQGRVTITADKSTSTAYMELSSLRAEDTAVYYCARRGNYYGSGSPDVFDIWGQGTMVTVSS

SEQ ID NO: 24 - protein, 107 aa, homo sapiens; identical to SEQ ID NO: 22

SEQ ID NO: 25 - protein, 124 aa, homo sapiens
QVQLVQSGAEVKKPGSSVKVSCKASGGTFSSYAINWMRQAPGQGLEWMGRIIPIFGIVNYAQKFQGRVTLTADKSTSTAYMELSSLRSEDTAVYYCARRGNYYGSGSPDVFDIWGQGTMVTVSS

SEQ ID NO: 26 - protein, 107 aa, homo sapiens; identical to SEQ ID NO: 22

SEQ ID NO: 27 - protein, 124 aa, homo sapiens; identical to SEQ ID NO: 25

SEQ ID NO: 28 - protein, 106 aa, homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWLTFGGGTKVEIK

SEQ ID NO: 29 - protein, 124 aa, homo sapiens
QVQLVQSGAEVKKPGSSVKVSCKASGGTFSSYAISWVRQAPGQGLEWMGRIIPIFGIANYAQKFQGRVTITADKSTSTAYMELSSLRSEDTAVYYCARRGNYYGSGSPDVFDIWGQGTMVTVSS

SEQ ID NO: 30 - protein, 107 aa, homo sapiens; identical to SEQ ID NO: 22

SEQ ID NO: 31 - protein, 123 aa, homo sapiens
QVQLQQWGAGLLKPSETLSLTCAIDGGSFSGYYWSWIRQPPGKGLEWIGEISHSGRTNYNPSLKSRVTISIDTSKNQFSLKLSSVAAADTAVYYCARFITMIRGAIITHFDYWGQGALVTVSS

SEQ ID NO: 32 - protein, 123 aa, homo sapiens
QVQLQQWGAGLLKPSETLSLTCAIDGGSFSGYYWSWIRQPPGKGLEWIGEISHSGRTNYNPSLKSRVTISIDTSKNQFSLKLSSVAAADTAVYYCARFITLIRGAIITHFDYWGQGALVTVSS

SEQ ID NO: 33 - protein, 107 aa, homo sapiens; identical to SEQ ID NO: 20

SEQ ID NO: 34 - protein, 120 aa, homo sapiens
QVQLVESGGGVVQPGRSLRLSCAASGFSFSTYAMHWVRQAPGKGLEWVAVISYDGDNKYSADSVKGRFTISRDNSKNTLYLQMNSLRAEDTAVYYCARGRKLGIDAFDIWGQGTMVTVSS

SEQ ID NO: 35 - protein, 107 aa, homo sapiens
AIQLTQSPSSLSASVGDRVTITCRASQGISSALAWYQQKPGKAPKLLIYDASSLESGVPSRFSGSGSGTDFTLTISGLQPEDFATYYCQQFNSYPFTFGPGTKVDIK

SEQ ID NO: 36 - protein, 8 aa, homo sapiens
GFTFSSYA

SEQ ID NO: 37 - protein, 8 aa, homo sapiens
TSGSGAST

SEQ ID NO: 38 - protein, 9 aa, homo sapiens
AKIWIAFDI

SEQ ID NO: 39 - protein, 7 aa, homo sapiens
QSVSSSY

SEQ ID NO: 40 - protein, 9 aa, homo sapiens
QQYGSSPYT

SEQ ID NO: 41 - protein, 8 aa, homo sapiens
GFTFSSYA

SEQ ID NO: 42 - protein, 8 aa, homo sapiens
ISISGAST

SEQ ID NO: 43 - protein, 13 aa, homo sapiens
RGYSGYVYDAFDI

SEQ ID NO: 44 - protein, 6 aa, homo sapiens
QGISNW

SEQ ID NO: 45 - protein, 9 aa, homo sapiens
QQYNSYPLT

SEQ ID NO: 46 - protein, 8 aa, homo sapiens
GFTFSSYA

SEQ ID NO: 47 - protein, 8 aa, homo sapiens
ISISGGST

SEQ ID NO: 48 - protein, 13 aa, homo sapiens
RGYSGYVYDAFDF

SEQ ID NO: 49 - protein, 6 aa, homo sapiens
QGISNW

SEQ ID NO: 50 - protein, 9 aa, homo sapiens
QQYNSYPLT

SEQ ID NO: 51 - protein, 8 aa, homo sapiens
GFTFSSYA

SEQ ID NO: 52 - protein, 8 aa, homo sapiens
ISIGGGNA

SEQ ID NO: 53 - protein, 14 aa, homo sapiens
AKPGFIMVRGPLDY

SEQ ID NO: 54 - protein, 14 aa, homo sapiens
AKPGFILVRGPLDY

SEQ ID NO: 55 - protein, 7 aa, homo sapiens
QSVSNSY

SEQ ID NO: 56 - protein, 9 aa, homo sapiens
QQYGSSPYT

SEQ ID NO: 57 - protein, 8 aa, homo sapiens
GFTFSSYA

SEQ ID NO: 58 - protein, 8 aa, homo sapiens
ISVSGGST

SEQ ID NO: 59 - protein, 18 aa, homo sapiens
AKEGYIWFGESLSYAFDI

SEQ ID NO: 60 - protein, 7 aa, homo sapiens
QSVSSSY

SEQ ID NO: 61 - protein, 8 aa, homo sapiens
QQYGRSFT

SEQ ID NO: 62 - protein, 8 aa, homo sapiens
GFTFSNYA

SEQ ID NO: 63 - protein, 8 aa, homo sapiens
ISVSGGST

SEQ ID NO: 64 - protein, 18 aa, homo sapiens
AKEGYIWFGESLSYAFDI

SEQ ID NO: 65 - protein, 7 aa, homo sapiens
QSVSSSY

SEQ ID NO: 66 - protein, 8 aa, homo sapiens
QQYGRSFT

SEQ ID NO: 67 - protein, 8 aa, homo sapiens
GFTFSSYA

SEQ ID NO: 68 - protein, 8 aa, homo sapiens
ISVSGGST

SEQ ID NO: 69 - protein, 18 aa, homo sapiens
AKEGYIWFGESLSYAFDI

SEQ ID NO: 70 - protein, 7 aa, homo sapiens
QSVSSSY

SEQ ID NO: 71 - protein, 8 aa, homo sapiens
QQYGRSFT

SEQ ID NO: 72 - protein, 8 aa, homo sapiens
GGSFSGYY

SEQ ID NO: 73 - protein, 5 aa, homo sapiens
INQSG

SEQ ID NO: 74 - protein, 7 aa, homo sapiens
IQQSGST

SEQ ID NO: 75 - protein, 11 aa, homo sapiens
ASGNWDHFFDY

SEQ ID NO: 76 - protein, 6 aa, homo sapiens
QGISSW

SEQ ID NO: 77 - protein, 9 aa, homo sapiens
QQAKSFPWT

SEQ ID NO: 78 - protein, 8 aa, homo sapiens
GGSFSGYH

SEQ ID NO: 79 - protein, 7 aa, homo sapiens
ISHSGRT

SEQ ID NO: 80 - protein, 17 aa, homo sapiens
ASFITMIRGTIITHFDY

SEQ ID NO: 81 - protein, 6 aa, homo sapiens
QGISSW

SEQ ID NO: 82 - protein, 9 aa, homo sapiens
QQYHSYPYT

SEQ ID NO: 83 - protein, 8 aa, homo sapiens
GGTFSSYA

SEQ ID NO: 84 - protein, 8 aa, homo sapiens
IIPIFGIA

SEQ ID NO: 85 - protein, 17 aa, homo sapiens
ARRGDYYGSGSPDVFDI

SEQ ID NO: 86 - protein, 7 aa, homo sapiens
QSVSSSY

SEQ ID NO: 87 - protein, 8 aa, homo sapiens
QQYGSSYT

SEQ ID NO: 88 - protein, 8 aa, homo sapiens
GGTFSSYA

SEQ ID NO: 89 - protein, 8 aa, homo sapiens
IIPIFGIA

SEQ ID NO: 90 - protein, 17 aa, homo sapiens
ARRGNYYGSGSPDVFDI

SEQ ID NO: 91 - protein, 7 aa, homo sapiens
QSVSSSY

SEQ ID NO: 92 - protein, 8 aa, homo sapiens
QQYGSSYT

SEQ ID NO: 93 - protein, 8 aa, homo sapiens
GGTFSSYA

SEQ ID NO: 94 - protein, 8 aa, homo sapiens
IIPIFGIV

SEQ ID NO: 95 - protein, 17 aa, homo sapiens
ARRGNYYGSGSPDVFDI

SEQ ID NO: 96 - protein, 7 aa, homo sapiens
QSVSSSY

SEQ ID NO: 97 - protein, 8 aa, homo sapiens
QQYGSSYT

SEQ ID NO: 98 - protein, 8 aa, homo sapiens
GGTFSSYA

SEQ ID NO: 99 - protein, 8 aa, homo sapiens
IIPIFGIV

SEQ ID NO: 100 - protein, 17 aa, homo sapiens
ARRGNYYGSGSPDVFDI

SEQ ID NO: 101 - protein, 6 aa, homo sapiens
QSVSSY

SEQ ID NO: 102 - protein, 8 aa, homo sapiens
QQRSNWLT

SEQ ID NO: 103 - protein, 8 aa, homo sapiens
GGTFSSYA

SEQ ID NO: 104 - protein, 8 aa, homo sapiens
IIPIFGIA

SEQ ID NO: 105 - protein, 18 aa, homo sapiens
ARRGNYYGSGSPDVFDIS

SEQ ID NO: 106 - protein, 7 aa, homo sapiens
QSVSSSY

SEQ ID NO: 107 - protein, 8 aa, homo sapiens
QQYGSSYT

SEQ ID NO: 108 - protein, 8 aa, homo sapiens
GGSFSGYY

SEQ ID NO: 109 - protein, 7 aa, homo sapiens
ISHSGRT

SEQ ID NO: 110 - protein, 17 aa, homo sapiens
ARFITMIRGAIITHFDY

SEQ ID NO: 111 - protein, 17 aa, homo sapiens
ARFITLIRGAIITHFDY

SEQ ID NO: 112 - protein, 6 aa, homo sapiens
QGISSW

SEQ ID NO: 113 - protein, 9 aa, homo sapiens
QQYHSYPYT

SEQ ID NO: 114 - protein, 8 aa, homo sapiens
GFSFSTYA

SEQ ID NO: 115 - protein, 8 aa, homo sapiens
ISYDGDNK

SEQ ID NO: 116 - protein, 13 aa, homo sapiens
ARGRKLGIDAFDI

SEQ ID NO: 117 - protein, 6 aa, homo sapiens
QGISSA

SEQ ID NO: 118 - protein, 9 aa, homo sapiens
QQFNSYPFT

SEQ ID NO: 119 - protein, 8 aa, homo sapiens
  MISC_FEATURE (6)..(6): Wherein X is A or G
ISISGXST

SEQ ID NO: 120 - protein, 13 aa, homo sapiens
  Misc (13)..(13): Wherein X is I of F
RGYSGYVYDAFDX

SEQ ID NO: 121 - protein, 8 aa, homo sapiens
  MISC_FEATURE (8)..(8): Wherein X is I or F
GGSFSGYX

SEQ ID NO: 122 - protein, 17 aa, homo sapiens
  MISC_FEATURE (2)..(2): Wherein X is S or R
  MISC_FEATURE (10)..(10): Wherein X is T or A
AXFITMIRGXIITHFDY

SEQ ID NO: 123 - protein, 8 aa, homo sapiens
  MISC_FEATURE (6)..(6): Wherein X is S or N
GFTFSXYA

SEQ ID NO: 124 - protein, 8 aa, homo sapiens
ISVSGGST

SEQ ID NO: 125 - protein, 18 aa, homo sapiens
AKEGYIWFGESLSYAFDI

SEQ ID NO: 126 - protein, 8 aa, homo sapiens
  MISC_FEATURE (8)..(8): Wherein X is A or V
IIPIFGIX

SEQ ID NO: 127 - protein, 17 aa, homo sapiens
  MISC_FEATURE (5)..(5): Wherein X is D or N
ARRGXYYGSGSPDVFDI

SEQ ID NO: 128 - protein, 7 aa, homo sapiens
  MISC_FEATURE (4)..(4): Wherein X is S or deleted
QSVXSSY

SEQ ID NO: 129 - protein, 8 aa, homo sapiens
  MISC_FEATURE (3)..(3): Wherein X is R or Y
  MISC_FEATURE (4)..(4): Wherein X is Sor G
  MISC_FEATURE (4)..(4): Wherein X is Sor G
  MISC_FEATURE (5)..(5): Wherein X is N or S
  MISC_FEATURE (6)..(6): Wherein X is W or S
  MISC_FEATURE (6)..(6): Wherein X is W or S
  MISC_FEATURE (7)..(7): Wherein X is L or Y
QQXXXXXT

SEQ ID NO: 130 - protein, 894 aa, homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 131 - protein, 904 aa, Mus Musculus
MAWRCPRMGRVPLAWCLALCGWACMYPYDVPDYAAHKDTQTEAGSPFVGNPGNITGARGLTGTLRCELQVQGEPPEVVWLRDGQILELADNTQTQVPLGEDWQDEWKVVSQLRISALQLSDAGEYQCMVHLEGRTFVSQPGFVGLEGLPYFLEEPEDKAVPANTPFNLSCQAQGPPEPVTLLWLQDAVPLAPVTGHSSQHSLQTPGLNKTSSFSCEAHNAKGVTTSRTATITVLPQRPHHLHVVSRQPTELEVAWTPGLSGIYPLTHCNLQAVLSDDGVGIWLGKSDPPEDPLTLQVSVPPHQLRLEKLLPHTPYHIRISCSSSQGPSPWTHWLPVETTEGVPLGPPENVSAMRNGSQVLVRWQEPRVPLQGTLLGYRLAYRGQDTPEVLMDIGLTREVTLELRGDRPVANLTVSVTAYTSAGDGPWSLPVPLEPWRPGQGQPLHHLVSEPPPRAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 132 - protein, 894 aa, homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDKAVPANTPFNLSCQAQGPPEPVTLLWLQDAVPLAPVTGHSSQHSLQTPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 133 - protein, 894 aa, homo sapiens; identical to SEQ ID NO: 132

SEQ ID NO: 134 - protein, 894 aa, homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQRPHHLHVVSRQPTELEVAWTPGLSGIYPLTHCNLQAVLSDDGVGIWLGKSDPPEDPLTLQVSVPPHQLRLEKLLPHTPYHIRISCSSSQGPSPWTHWLPVETTEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 135 - protein, 894 aa, homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENVSAMRNGSQVLVRWQEPRVPLQGTLLGYRLAYRGQDTPEVLMDIGLTREVTLELRGDRPVANLTVSVTAYTSAGDGPWSLPVPLEPWRPGQGQPLHHLVSEPPPRAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 136 - protein, 124 aa, homo sapiens
EVQLLESGGGLVQPGGSLRLSCAASGFTFSSYAMNWVRQAPGKGLEWVSGISGSGGHTYHADSVKGRFTISRDNSKNTLYLQMNSLRAEDTAVYYCAKDRYDILTGYYNLLDYWGQGTLVTVSS

SEQ ID NO: 137 - protein, 8 aa, homo sapiens
GFTFSSYA

SEQ ID NO: 138 - protein, 8 aa, homo sapiens
ISGSGGHT

SEQ ID NO: 139 - protein, 17 aa, homo sapiens
AKDRYDILTGYYNLLDY

SEQ ID NO: 140 - protein, 107 aa, homo sapiens
DIQMTQSPSSLSASVGDRVTITCRASQGISSWLAWYQQKPEEAPKSLIYAASSLQSGVPSRFSGSGSGTDFTLTISSLQPEDFATYYCQQYNSYPLTFGGGAKVEIK

SEQ ID NO: 141 - protein, 6 aa, homo sapiens
QGISSW

SEQ ID NO: 142 - protein, 9 aa, homo sapiens
QQYNSYPLT

SEQ ID NO: 143 - protein, 123 aa, homo sapiens
QVQLVQSGAEVKKPGASVKVSCKASGYAFTGYGISWVRQAPGQGLEWIGWISAYNGNTNYVQNLQDRVTMTTDTSTSTAYMELRSLRSDDTAVYYCARDHISMLRGIIIRNYWGQGTLVTVSS

SEQ ID NO: 144 - protein, 108 aa, homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSSWPRLTFGGGTKVEIK

SEQ ID NO: 145 - protein, 124 aa, homo sapiens
QVQLVQSGAEVKKPGSSVKVSCKASGGTFSRYAISWVRQAPGQGLEWMGRIIPIVGIANYAQKFQGRVTLTADKSTSTAYMELSSLRSEDTAVYYCAREAGYSSSWYAEYFQHWGQGTLVTVSS

SEQ ID NO: 146 - protein, 108 aa, homo sapiens
EIVLTQSPGTLSLSPGERATLSCRASQSVSSNYLAWYQQKPGQAPRLLIYGASSRATGFPDRFSGSGSGTDFTLTISRLEPEDFAVYYCQQYGSSPYTFGQGTKLEIK

SEQ ID NO: 147 - protein, 893 aa, Macaca fascicularis
AWRCPRMGRVPLAWCLALCGWVCMAPRGTQAEESPFVGNPGNITGARGLTGTLRCQLQVQGEPPEVHWLRDGQILELADSTQTQVPLGEDEQDDWIVVSQLRIASLQLSDAGQYQCLVFLGHQNFVSQPGYVGLEGLPYFLEEPEDRTVAANTPFNLSCQAQGPPEPVDLLWLQDAVPLATAPGHGPQRNLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTLQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKETSAPAFSWPWWYILLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQLDPKDSCSCLTSAEVHPAGRYVLCPSTAPSPAQPADRGSPAAPGQEDGA